(19) United States
(12) Patent Application Publication
(10) Pub. No.: US 2024/0358611 A1
(43) Pub. Date: Oct. 31, 2024

(54) COMPOSITIONS

(71) Applicant: SYMRISE AG, Holzminden (DE)

(72) Inventors: Benoît JOIN, Holzminden (DE); Judith MINAKAR, Neustadt an der Weinstraße (DE); Sven SIEGEL, Höxter (DE); Arnold MACHINEK, Holzminden (DE); Sebastian AHLES, Holzminden (DE)

(21) Appl. No.: 18/684,024

(22) PCT Filed: Aug. 16, 2022
(86) PCT No.: PCT/EP2022/072808

§ 371 Date: Feb. 15, 2024

(30) Foreign Application Priority Data
Aug. 16, 2021 (WO) PCT/EP2021/072697

(51) Int. Cl. A61K 8/49; A23G 3/36; A23G 4/06; A23L 3/3544; A23L 27/00; A61K 8/34; A61Q 5/00; A61Q 5/02; A61Q 5/06; A61Q 5/12; A61Q 9/02; A61Q 11/00; A61Q 15/00; A61Q 17/00; A61Q 17/02; A61Q 19/00; A61Q 19/08; A61Q 19/10; C11D 3/00; C11D 3/28

(52) U.S. Cl. CPC A61K 8/4946; A23G 3/36; A23G 4/06; A23L 3/3544; A23L 27/84; A23L 27/88; A61K 8/34; A61Q 5/006; A61Q 5/02; A61Q 5/06; A61Q 5/12; A61Q 9/02; A61Q 11/00; A61Q 15/00; A61Q 17/005; A61Q 17/02; A61Q 19/007; A61Q 19/08; A61Q 19/10; C11D 3/001; C11D 3/28; C11D 2111/12; C11D 2111/14

(57) ABSTRACT

Suggested are compositions comprising substituted azacycles on one hand and physiological cooling or warming agents, flavors and/or fragrances on the other for masking, enhancing and/or boosting flavors, improving soothing ability, and improving antimicrobial stability.

### AREA OF INVENTION

The present invention refers to the area of preservation and concerns new composition comprising specific multi-purpose mixtures with anti-microbial activity, flavor boosting activity, flavor masking activity and soothing activity.

### BACKGROUND OF THE INVENTION

Cosmetic products are generally manufactured under sterile conditions to prevent contamination. In the case of foodstuffs, the requirements are even more stringent; dairy products, for example, must be subjected to high-temperature heating (“pasteurization”) before they can be marketed at all.

However, in the course of their use, the above-mentioned products are regularly contaminated and chemically altered by microorganisms, especially bacteria and fungi, so that they are subsequently no longer usable or edible, but on the contrary represent a danger to healthy organisms. This can happen through the spatula, which has a contamination and then transfers it to the cream, or bacteria are simply transferred through the air.

Even though there is a growing market for products that are untreated and do not contain substances that kill microorganisms, there is just as much interest in products that have a sufficiently long shelf life after the package is broken open. For this reason, preservatives are added to cosmetics and food products. By their very nature, preservatives are generally considered to be hazardous for contact with human skin and especially mucous membranes, because a chemical that is capable of killing microorganisms is always suspected of triggering harmful processes in the human organism. The list of approved preservatives is therefore shrinking from year to year at the same rate as critical studies are revealing dangers for previously common substances.

Apart from new preservatives that are as natural as possible, which still need to be developed, there is great interest in both the cosmetics and food industries in ways of reducing the concentration of preservatives in a formulation without sacrificing protection against microbial attack. A first concept is to use synergistic mixtures of different preservatives. Another approach pursues the possibility of identifying multifunctional substances or mixtures of substances that, in addition to their primary properties, are also antimicrobially active, at least to a small extent. To the extent that these substances contribute to the antimicrobial properties of a formulation, the amount of actual preservative can be reduced.

Another widespread problem, both in the field of cosmetics and cleaning products and in foodstuffs, is to mask the unpleasant odor or taste of ingredients that cannot be replaced in the formulation. A typical example is fluorine compounds used in dental care products. There is also a constant need for substances that perform important tasks in the above-mentioned preparations, such as for example providing a physiological cooling effect on the skin or mucous membrane, while also exhibiting other properties that are important for the formulation. Special interest is shown in preparations which, preferably even in very small quantities, are capable of intensifying positive taste or odor impressions or of bringing them to bear in the first place.

### RELEVANT PRIOR ART

EP 3315492 A1 (KISSEI) discloses novel pyrazol derivatives according to the following formula

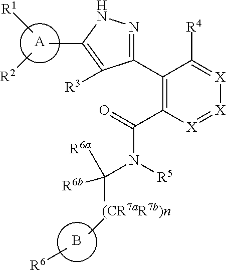

showing TRPM8 inhibition activity and are useful particularly for treating or preventing diseases or symptoms caused by hyper-excitability or disorder of afferent neurons.

WO 2021 074281 A1 (GIVAUDAN) discloses substituted azacycles and their use as TMPR8 modulators for achieving a cooling effect on skin. The compounds are also disclosed together with other physiological cooling agents and flavors for enhancing the cooling properties.

U.S. Pat. No. 8,143,259 (JANSSEN) refers to cold menthol receptor antagonists according to the following formula

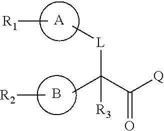

for treating various disorders including pain.
Reference is also made to the paper “Transient Receptor Potential Melastin 8 Channel modulation: Cool entryway for treating pain and cancer” by PEREZ DE VEGA et al published in J. Med. Chem 59(22), p 10006-10029 (2016).

### OBJECT OF THE INVENTION

Therefore, it has been the object of the present invention providing compositions which are commonly used in cosmetic products or foodstuffs and which additionally possess appreciable antimicrobial properties, so that their use can reduce the amount of preservative otherwise required for antimicrobial finishing, mask unpleasant flavors, particularly unpleasant flavors of fluorine derivatives and bitter compounds; and/or enhance and/or boost the performance of a flavor, a sweetener or a soothing agent.

### BRIEF DESCRIPTION OF THE INVENTION

A first object of the present invention refers to a composition comprising or consisting of
(a) at least one substituted azacycle according to formula (I), a salt or a solvate thereof

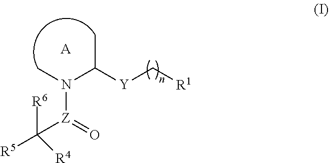

wherein
n is 0 or 1 and R¹ is selected from
(i) halogen,
(ii) C₆-C₁₀ aryl,
(iii) C₅-C₁₀ mono- or bicyclic heteroaryl wherein up to 2 C-atoms are replaced by at least one hetero atom independently selected from sulfur, nitrogen, and oxygen,
(iv) C₆-C₁₀ aryl substituted with up to four substituents independently selected from the group consisting of halogen; OH; C═N; NO₂; C₁-C₆ alky; C₁-C₆ alkyl comprising up to 5 halogen atoms; C₁-C₃ alkyl comprising up to 3 OH groups; C₂-C₆ alkenyl; C₁-C₆ alkoxy; C₁-C₆ alkoxy comprising up to 3 halogen atoms; C₁-C₃ alkoxy C₁-C₃ alkyl; C₃-C₇ cycloalkyl; —C(O)R¹⁰ wherein R¹⁰ is selected from C₁-C₃ alkyl; —OC(O)R¹¹ wherein R¹¹ is selected from H, and C₁-C₃ alkyl; —C(O)O—R¹² wherein R¹² is selected from hydrogen and C₁-C₃ alkyl; —(CH₂)ₘN(R¹³)R¹⁴ wherein m is 0 or 1, R¹³ is selected from hydrogen, C₁-C₃-alkyl, and —SOR¹⁵ wherein R¹⁵ is C₁-C₃-alkyl, and R¹⁴ is selected from hydrogen, C₁-C₃-alkyl, and —SO₂R¹⁶ wherein R¹⁶ is C₁-C₃-alkyl, or wherein R¹³ and 14 form together with the N atom to which they are attached morpholine, thiomorpholine, or 1,1-dioxothiomorpholine; —SR¹⁷ wherein R¹⁷ is selected from hydrogen and C₁-C₃ alkyl; and —S(O)₂R¹⁸ wherein R¹⁸ is selected from hydrogen and C₁-C₃ alkyl; with the proviso that when the aryl ring is substituted with two or more substituents, two substituents may form a cyclic ring together with the carbon atoms to which they are attached, and
(v) C₅-C₁₀ mono- or bicyclic heteroaryl wherein up to 2 C-atoms are replaced by a hetero atoms independently selected from sulfur, nitrogen, and oxygen, substituted with up to four substituents selected from the group consisting of halogen; OH (hydroxyl); C═N (cyano); NO₂ (nitro); C₁-C₆ alky; C₁-C₆ alky comprising up to 5 halogen atoms; C₂-C₆ alkenyl; C₁-C₆ alkoxy; C₁-C₆ alkoxy comprising up to 3 halogen atoms; C₁-C₃ alkoxy C₁-C₃ alkyl; C₃-C₇ cycloalkyl; —C(O)R²⁰ wherein R²⁰ is selected from C₁-C₃ alkyl; —OC(O)R²¹ wherein R²¹ is selected from H, and C₁-C₃ alkyl; —C(O)O—R²² wherein R²² is selected from hydrogen and C₁-C₃ alkyl; —(CH₂)ₘN(R²³)R²⁴ wherein m is 0 or 1, R²³ is selected from hydrogen, C—C alkyl, and —SOR²¹ wherein R^{21 24} 1₃₂ is C₁-C₃ alkyl, and R is selected from hydrogen, C^{26 26} 1-C₃ alkyl, and —SO₂R wherein R is C₁-C₃ alkyl, or wherein R²³ and R²⁴ form together with the N atom to which they are attached morpholine, thiomorpholine, or 1,1-dioxothiomorpholine; —SR²⁷ wherein R²⁷ is selected from hydrogen and C₁-C₃ alkyl; and —S(O)₂R²⁸ wherein R²⁸ is selected from hydrogen and C₁-C₃ alkyl; Y is a monocyclic or bicyclic, unsaturated or aromatic heterocyclic ring comprising one, two, three or four heteroatoms independently selected from nitrogen, sulfur and oxygen, wherein the ring is optionally mono-, di-, or tri-substituted with a group selected from halogen, methyl, ethyl, —N(R⁴⁰)R⁴¹, —CON(R⁴⁰)R⁴¹, —[CR⁴⁰R⁴¹]—C(O)OR⁴⁰, —C^{40 40 41} p (O)R, and —SO₂N(R)R wherein R⁴⁰ and R⁴¹ are independently selected from hydrogen and C₁-C₄ alkyl, and p is 0, 1, or 2; A is a 5 to 7 membered heterocyclic ring optionally comprising one additional hetero atom selected from oxygen and sulfur, wherein the heterocyclic ring A is optionally substituted by one or two groups selected from —OH and ═O; Z is either C, S or S(O);
and R⁴, R⁵ and R⁶ form together with the carbon atom to which they are attached a hydrocarbon group optionally comprising up to five hetero atoms selected from O, N, S, and F;
(b) at least one physiological cooling or warming agent and/or
(c) at least one aroma or flavoring compound, and/or
(d) at least one fragrance
   whereby the compounds are present in a ratio by weight (a):(b+c+d) of from about 1:1,500,00:1 to about 1:1, preferably from 1:500,000 to about 1:10. More preferably from about 1:100,000 to about 1:100, and most preferably from about 1:40,000 to about 1:500.

In a first embodiment the compositions according to the present invention include compound (a) and one additional additive, namely compound (b), compound (c) or compound (d). However, it is also possible having compositions with three or four components. For ternary mixtures the preferred ratios by weight (b:c), (b:d) or (c):(d) are from about 10:90 to about 90:10 and preferably from about 25:75 to about 75:25. For quaternary mixtures the preferred ratios by weight (b):(c):(d) are from about 30:30:40 to about 40:30:30.

Each of the sub-groups (i) to (v) represents an individually preferred embodiment of Compound (a).

Surprisingly, it was found that defined mixtures of representatives of substance class (a) with physiological cooling or warming substances, flavors and/or fragrances, possess moderate antimicrobial activity against the most important germs found in both cosmetic products and foods. If these mixtures are used, on the one hand they fulfill their actual task, namely, for example, to produce a cooling effect on the skin or to impart a certain flavor or fragrance to a particular product, but at the same time they also contribute to the antimicrobial stabilization of the products, so that the amount of preservatives actually required can be reduced. These findings are therefore particularly unexpected, since the substances tested alone do not possess any antimicrobial activity. At this point, therefore, a synergy can also be observed. Moreover they possess the ability to mask unpleasant flavors, particularly unpleasant flavors of fluorine derivatives and bitter compounds; and/or enhance and/or boost the performance of a flavor, a sweetener or a soothing agent.

### Substituted Azacyles

Substituted azacycles forming group (a) belong to the group of TRMP8 modulators. The structures and their synthesis is known from WO 2021074281 A1 which is hereby incorporated by reference. Suitable azacycles encompass the following species:
(2-(4-phenyl-1H-imidazol-2-yl)piperidin-1-yl)(1,2,3-thiadiazol-5-yl)methanone
(2S)-2-methyl-1-[2-[5-(p-tolyl)-1H-imidazol-2-yl]-1-piperidyl]butan-1-one
2-methylsulfanyl-1-[(1R)-2-[5-(p-tolyl)-1H-imidazol-2-yl]-1-piperidyl]-propan-1-one
2-methyl-1-[2-[5-(p-tolyl)-1H-imidazol-2-yl]-1-piperidyl]butan-1-one
(2-(4-phenyl-1H-imidazol-2-yl)piperidin-1-yl)(thietan-3-yl)methanone,
(2,2-dimethylcyclopropyl)(2-(4-phenyl-1H-imidazol-2-yl)piperidin-1-yl)methanone,
(2-ethoxycyclopropyl)(2-(4-phenyl-1H-imidazol-2-yl)piperidin-1-yl)methanone,
(2-methoxycyclopropyl)(2-(4-phenyl-1H-imidazol-2-yl)piperidin-1-yl)methanone,
(2-methylcyclopropyl)(2-(4-phenyl-1H-imidazol-2-yl)piperidin-1-yl)methanone,
(2S)-2-methyl-1-(2-(5-(p-tolyl)-1H-imidazol-2-yl)piperidin-1-yl)butan-1-one,
(5-methyltetrahydrofuran-3-yl)(2-(4-phenyl-1H-imidazol-2-yl)piperidin-1-yl)methanone,
(E)-1-(2-(5-(3,4-difluorophenyl)-1H-imidazol-2-yl)piperidin-1-yl)-2-methylbut-2-en-1-one,
(E)-2-methyl-1-(2-(5-(p-tolyl)-1H-imidazol-2-yl)piperidin-1-yl)but-2-en-1-one,
(E)-2-methyl-1-(2-(5-(p-tolyl)-1H-imidazol-2-yl)piperidin-1-yl)pent-3-en-1-one,
(R)-2-(methylthio)-1-((R)-2-(5-(p-tolyl)-1H-imidazol-2-yl)piperidin-1-yl)propan-1-one.
(S)-2-(methylthio)-1-((R)-2-(5-(p-tolyl)-1H-imidazol-2-yl)piperidin-1-yl)propan-1-one,
(S)-2-(methylthio)-1-((S)-2-(5-(p-tolyl)-1H-imidazol-2-yl)piperidin-1-yl)propan-1-one,
(S)-2-methyl-1-((R)-2-(5-(p-tolyl)-1H-imidazol-2-yl)piperidin-1-yl)butan-1-one,
(tetrahydrofuran-3-yl)(2-(5-(p-tolyl)-1H-imidazol-2-yl)piperidin-1-yl)methanone,
1-(2-(1H-benzo[d]imidazol-2-yl)piperidin-1-yl)-2-(methylthio)propan-1-one,
1-(2-(4-(1-methyl-1H-pyrazol-4-yl)-1H-imidazol-2-yl)piperidin-1-yl)-2-(methylthio)propan-1-one,
1-(2-(4-(2,2-difluorobenzo[d][1,3]dioxol-5-yl)-1H-imidazol-2-yl)piperidin-1-yl)-2-(methyl-thio)propan-1-one,
1-(2-(4-(2,3-dihydrobenzofuran-5-yl)-1H-imidazol-2-yl)piperidin-1-yl)-2-(methylthio)propan-1-one,
1-(2-(4-(2,3-dihydrobenzofuran-6-yl)-1H-imidazol-2-yl)piperidin-1-yl)-2-(methylthio)propan-1-one,
1-(2-(4-(2,5-dimethylphenyl)-1H-imidazol-2-yl)piperidin-1-yl)-2-(methylthio)propan-1-one
1-(2-(4-(2-chlorophenyl)-1H-imidazol-2-yl)piperidin-1-yl)-2-(methylthio)propan-1-one,
1-(2-(4-(2-cyclopropyloxazol-5-yl)-1H-imidazol-2-yl)piperidin-1-yl)-2-(methylthio)propan-1-one,
1-(2-(4-(2-fluorophenyl)-1H-imidazol-2-yl)piperidin-1-yl)-2-(methylthio)propan-1-one.
1-(2-(4-(2-hydroxyphenyl)-1H-imidazol-2-yl)piperidin-1-yl)-2-(methylthio)propan-1-one,
(2-(4-(3-((dimethylamino)methyl)phenyl)-1H-imidazol-2-yl)piperidin-1-yl)-2-(methyl-thio)propan-1-one,
1-(2-(4-(3,5-difluorophenyl)-1H-imidazol-2-yl)piperidin-1-yl)-2-(methylthio)propan-1-one,
1-(2-(4-(3,5-dimethylisoxazol-4-yl)-1H-imidazol-2-yl)piperidin-1-yl)(methylthio)propan-1-one,
1-(2-(4-(3-chloro-4-methylphenyl)-1H-imidazol-2-yl)piperidin-1-yl)-2-(methylthio)propan-1-one,
1-(2-(4-(3-methylfuran-2-yl)-1H-imidazol-2-yl)piperidin-1-yl)-2-(methylthio)propan-1-one.
1-(2-(4-(4-((1,1-dioxidothiomorpholino)methyl)phenyl)-1H-imidazol-2-yl)piperidin-1-yl)-2-(methylthio)propan-1-one,
1-(2-(4-(4-(1-hydroxyethyl)phenyl)-1H-imidazol-2-yl)piperidin-1-yl)-2-(methylthio)propan-1-one,
1-(2-(4-(4-(difluoromethoxy)-3-fluorophenyl)-1H-imidazol-2-yl)piperidin-1-yl)-2-(methyl-thio)propan-1-one,
1-(2-(4-(4-(difluoromethyl)thiophen-3-yl)-1H-imidazol-2-yl)piperidin-1-yl)-2-(methyl-thio)propan-1-one,
1-(2-(4-(4-(methylsulfonyl)phenyl)-1H-imidazol-2-yl)piperidin-1-yl)-2-(methylthio)propan-1-one,
1-(2-(4-(4-(tert-butyl)phenyl)-1H-imidazol-2-yl)piperidin-1-yl)-2-(methylthio)propan-1-one,
1-(2-(4-(4-cyclobutylphenyl)-1H-imidazol-2-yl)piperidin-1-yl)-2-(methylthio)propan-1-one,
1-(2-(4-(4-cyclopropyl-2-fluorophenyl)-1H-imidazol-2-yl)piperidin-1-yl)-2-(methylthio)pro-pan-1-one,
1-(2-(4-(4-fluorophenyl)-1H-imidazol-2-yl)piperidin-1-yl)-2-(methylthio)propan-1-one,
1-(2-(4-(4-hydroxyphenyl)-1H-imidazol-2-yl)piperidin-1-yl)-2-(methylthio)propan-1-one,
1-(2-(4-(4-isopropylphenyl)-1H-imidazol-2-yl)piperidin-1-yl)-2-(methylthio)propan-1-one,
1-(2-(4-(4-methyl-3-(trifluoromethyl)phenyl)-1H-imidazol-2-yl)piperidin-1-yl)-2-(methylthio)propan-1-one,
1-(2-(4-(5-((dimethylamino)methyl)thiophen-2-yl)-1H-imidazol-2-yl)piperidin-1-yl)-2-(methylthio)propan-1-one,
1-(2-(4-(5-methylfuran-2-yl)-1H-imidazol-2-yl)piperidin-1-yl)-2-(methylthio)propan-1-one,
1-(2-(4-(5-methylpyridin-3-yl)-1H-imidazol-2-yl)piperidin-1-yl)-2-(methylthio)propan-1-one,
1-(2-(4-(6-methylpyridin-3-yl)-1H-imidazol-2-yl)piperidin-1-yl)-2-(methylthio)propan-1-one,
1-(2-(4-(benzo[b]thiophen-6-yl)-1H-imidazol-2-yl)piperidin-1-yl)-2-(methylthio)propan-1-one
1-(2-(4-methyl-5-phenyl-1H-imidazol-2-yl)piperidin-1-yl)-2-(methylthio)propan-1-one,
1-(2-(4-phenyl-1H-imidazol-2-yl)piperidin-1-yl)-2-((trifluoromethyl)thio)ethan-1-one,
1-(2-(4-phenyl-1H-imidazol-2-yl)piperidin-1-yl)butan-1-one,
1-(2-(4-phenyl-1H-imidazol-2-yl)piperidin-1-yl)hexa-3,5-dien-1-one,
1-(2-(4-phenyl-1H-imidazol-2-yl)piperidin-1-yl)pent-4-en-1-one,
1-(2-(4-phenyl-1H-imidazol-2-yl)piperidin-1-yl)propan-1-one,
1-(2-(5-(2,3-difluorophenyl)-1H-imidazol-2-yl)piperidin-1-yl)-2-(methylthio)propan-1-one,
1-(2-(5-(2,4-difluorophenyl)-1H-imidazol-2-yl)piperidin-1-yl)-2-(methylthio)propan-1-one,
1-(2-(5-(2,5-difluorophenyl)-1H-imidazol-2-yl)piperidin-1-yl)-2-(methylthio)propan-1-one,
1-(2-(5-(2-fluoro-4-methoxyphenyl)-1H-imidazol-2-yl)piperidin-1-yl)-2-(methylthio)propan-1-one,
1-(2-(5-(2-fluoro-4-methylphenyl)-1H-imidazol-2-yl)piperidin-1-yl)-2-(methylthio)propan-1-one,
1-(2-(5-(2-methoxypyrimidin-5-yl)-1H-imidazol-2-yl)piperidin-1-yl)-2-(methylthio)propan-1-one,
1-(2-(5-(3-(methylamino)phenyl)-1H-imidazol-2-yl)piperidin-1-yl)-2-(methylthio)propan-1-one,
1-(2-(5-(3,4-difluorophenyl)-1H-imidazol-2-yl)piperidin-1-yl)-2-(methylthio)propan-1-one,
1-(2-(5-(3,4-difluorophenyl)-1H-imidazol-2-yl)piperidin-1-yl)-2-methylbut-3-en-1-one,
1-(2-(5-(3,4-dimethoxyphenyl)-1H-imidazol-2-yl)piperidin-1-yl)-2-(methylthio)propan-1-one.
1-(2-(5-(3,4-dimethylphenyl)-1H-imidazol-2-yl)piperidin-1-yl)-2-(methylthio)propan-1-one,
1-(2-(5-(3-fluoro-4-(methoxymethyl)phenyl)-1H-imidazol-2-yl)piperidin-1-yl)-2-(methyl-thio)propan-1-one
1-(2-(5-(3-fluoro-4-methylphenyl)-1H-imidazol-2-yl)piperidin-1-yl)-2-(methylthio)propan-1-one,
1-(2-(5-(3-fluorophenyl)-1H-imidazol-2-yl)piperidin-1-yl)-2-(methylthio)propan-1-one,
1-(2-(5-(3-methoxyisothiazol-4-yl)-1H-imidazol-2-yl)piperidin-1-yl)-2-(methylthio)propan-1-one,
1-(2-(5-(3-methoxyphenyl)-1H-imidazol-2-yl)piperidin-1-yl)-2-(methylthio)propan-1-one,
1-(2-(5-(4-((methylamino)methyl)phenyl)-1H-imidazol-2-yl)piperidin-1-yl)-2-(methyl-thio)propan-1-one,
1-(2-(5-(4-(2-methoxyethyl)phenyl)-1H-imidazol-2-yl)piperidin-1-yl)-2-(methylthio)propan-1-one,
1-(2-(5-(4-(aminomethyl)phenyl)-1H-imidazol-2-yl)piperidin-1-yl)-2-(methylthio)propan-1-one,
1-(2-(5-(4-(dimethylamino)phenyl)-1H-imidazol-2-yl)piperidin-1-yl)-2-(methylthio)propan-1-one,
1-(2-(5-(4-(hydroxymethyl)phenyl)-1H-imidazol-2-yl)piperidin-1-yl)-2-(methylthio)propan-1-one,
1-(2-(5-(4-acetylphenyl)-1H-imidazol-2-yl)piperidin-1-yl)-2-(methylthio)propan-1-one,
1-(2-(5-(4-aminophenyl)-1H-imidazol-2-yl)piperidin-1-yl)-2-(methylthio)propan-1-one,
1-(2-(5-(4-chloro-3-fluorophenyl)-1H-imidazol-2-yl)piperidin-1-yl)-2-(methylthio)propan-1-one,
1-(2-(5-(4-chlorophenyl)-1H-imidazol-2-yl)piperidin-1-yl)-2-(methylthio)propan-1-one,
1-(2-(5-(4-ethylphenyl)-1H-imidazol-2-yl)piperidin-1-yl)-2-(methylthio)propan-1-one,
1-(2-(5-(4-fluorophenyl)-4H-1,2,4-triazol-3-yl)piperidin-1-yl)-2-(methylthio)propan-1-one,
1-(2-(5-(4-methoxy-2-methylphenyl)-1H-imidazol-2-yl)piperidin-1-yl)-2-(methylthio)propan-1-one,
1-(2-(5-(4-methoxyphenyl)-1H-imidazol-2-yl)piperidin-1-yl)-2-(methylthio)propan-1-one,
1-(2-(5-(4-methyl-6-(methylthio)pyridin-3-yl)-1H-imidazol-2-yl)piperidin-1-yl)-2-(methyl-thio)propan-1-one,
1-(2-(5-(5-(methoxymethyl)thiophen-3-yl)-1H-imidazol-2-yl)piperidin-1-yl)-2-(methyl-thio)propan-1-one,
1-(2-(5-(6-methoxypyridin-3-yl)-1H-imidazol-2-yl)piperidin-1-yl)-2-(methylthio)propan-1-one,
1-(2-(5-(benzo[d][1,3]dioxol-5-yl)-1H-imidazol-2-yl)piperidin-1-yl)-2-(methylthio)propan-1-one,
1-(2-(5-(p-tolyl)-1H-imidazol-2-yl)piperidin-1-yl)propan-1-one,
1-(2-(5-benzyl-1H-imidazol-2-yl)piperidin-1-yl)-2-(methylthio)propan-1-one,
1-(2-(5-bromo-1H-imidazol-2-yl)piperidin-1-yl)-2-(methylthio)propan-1-one,
2-(2-(1-(2-(methylthio)propanoyl)piperidin-2-yl)-1H-imidazol-5-yl)benzonitrile,
2-(2-(4-phenyl-1H-imidazol-2-yl)piperidine-1-carbonyl)cyclopropane-1-carbonitrile,
2-(5-methylfuran-2-yl)-1-(2-(4-phenyl-1H-imidazol-2-yl)piperidin-1-yl)propan-1-one,
2-(allylthio)-1-(2-(5-(p-tolyl)-1H-imidazol-2-yl)piperidin-1-yl)propan-1-one,
2-(dimethylamino)-1-(2-(4-phenyl-1H-imidazol-2-yl)piperidin-1-yl)butan-1-one,
2-(dimethylamino)-1-(2-(4-phenyl-1H-imidazol-2-yl)piperidin-1-yl)propan-1-one,
2-(ethylthio)-1-(2-(5-(p-tolyl)-1H-imidazol-2-yl)piperidin-1-yl)propan-1-one,
2-(furan-2-yl)-1-(2-(4-phenyl-1H-imidazol-2-yl)piperidin-1-yl)ethan-1-one,
2-(furan-2-yl)-1-(2-(4-phenyl-1H-imidazol-2-yl)piperidin-1-yl)propan-1-one,
2-(furan-3-yl)-1-(2-(4-phenyl-1H-imidazol-2-yl)piperidin-1-yl)ethan-1-one,
2-(furan-3-yl)-1-(2-(5-(p-tolyl)-1H-imidazol-2-yl)piperidin-1-yl)propan-1-one,
2-(methylamino)-N-(4-(2-(1-(2-(methylthio)propanoyl)piperidin-2-yl)-1H-imidazol-5-yl)phenyl)acetamide,
2-(methylsulfinyl)-1-(2-(5-(p-tolyl)-1H-imidazol-2-yl)piperidin-1-yl)propan-1-one,
2-(methylsulfonyl)-1-(2-(4-phenyl-1H-imidazol-2-yl)piperidin-1-yl)ethan-1-one,
2-(methylsulfonyl)-1-(2-(4-phenyl-1H-imidazol-2-yl)piperidin-1-yl)propan-1-one,
2-(methylthio)-1-(2-(2-(p-tolyl)-2H-tetrazol-5-yl)piperidin-1-yl)propan-1-one,
2-(methylthio)-1-(2-(3-phenyl-1,2,4-oxadiazol-5-yl)piperidin-1-yl)propan-1-one,
2-(methylthio)-1-(2-(4-(1-(trifluoromethyl)-1H-pyrazol-4-yl)-1H-imidazol-2-yl)piperidin-1-yl)propan-1-one,
2-(methylthio)-1-(2-(4-(4-(morpholinomethyl)phenyl)-1H-imidazol-2-yl)piperidin-1-yl)pro-pan-1-one,
2-(methylthio)-1-(2-(4-(5-(trifluoromethyl)furan-2-yl)-1H-imidazol-2-yl)piperidin-1-yl)pro-pan-1-one,
2-(methylthio)-1-(2-(4-(5-(trifluoromethyl)thiophen-2-yl)-1H-imidazol-2-yl)piperidin-1-yl)propan-1-one,
2-(methylthio)-1-(2-(4-(5-methylthiophen-3-yl)-1H-imidazol-2-yl)piperidin-1-yl)propan-1-one,
2-(methylthio)-1-(2-(4-(m-tolyl)-1H-imidazol-2-yl)piperidin-1-yl)propan-1-one,
2-(methylthio)-1-(2-(4-(o-tolyl)-1H-imidazol-2-yl)piperidin-1-yl)propan-1-one,
2-(methylthio)-1-(2-(4-(pyridin-3-yl)-1H-imidazol-2-yl)piperidin-1-yl)propan-1-one,
2-(methylthio)-1-(2-(4-(pyridin-4-yl)-1H-imidazol-2-yl)piperidin-1-yl)propan-1-one,
2-(methylthio)-1-(2-(4-(pyrimidin-5-yl)-1H-imidazol-2-yl)piperidin-1-yl)propan-1-one,
2-(methylthio)-1-(2-(4-(quinoxalin-6-yl)-1H-imidazol-2-yl)piperidin-1-yl)propan-1-one,
2-(methylthio)-1-(2-(4-thiophen-3-yl)-1H-imidazol-2-yl)piperidin-1-yl)propan-1-one,
2-(methylthio)-1-(2-(4-phenyl-1H-imidazol-2-yl)piperidin-1-yl)ethan-1-one,
2-(methylthio)-1-(2-(5-(2-(trifluoromethyl)phenyl)-1H-imidazol-2-yl)piperidin-1-yl)propan-1-one,
2-(methylthio)-1-(2-(5-(2,3,4-trifluorophenyl)-1H-imidazol-2-yl)piperidin-1-yl)propan-1-one,
2-(methylthio)-1-(2-(5-(2,4,5-trifluorophenyl)-1H-imidazol-2-yl)piperidin-1-yl)propan-1-one,
2-(methylthio)-1-(2-(5-(3-(trifluoromethyl)phenyl)-1H-imidazol-2-yl)piperidin-1-yl)propan-1-one,
2-(methylthio)-1-(2-(5-(4-(trifluoromethoxy)phenyl)-1H-imidazol-2-yl)piperidin-1-yl)propan-1-one,
2-(methylthio)-1-(2-(5-(4-(trifluoromethyl)phenyl)-1H-imidazol-2-yl)piperidin-1-yl)propan-1-one,
2-(methylthio)-1-(2-(5-(4-propylphenyl)-1H-imidazol-2-yl)piperidin-1-yl)propan-1-one,
2-(methylthio)-1-(2-(5-(4-vinylphenyl)-1H-imidazol-2-yl)piperidin-1-yl)propan-1-one,
2-(methylthio)-1-(2-(5-(5-methylthiophen-2-yl)-1H-imidazol-2-yl)piperidin-1-yl)propan-1-one,
2-(methylthio)-1-(2-(5-(p-tolyl)-1,3,4-oxadiazol-2-yl)piperidin-1-yl)propan-1-one,
2-(methylthio)-1-(2-(5-(p-tolyl)-1H-imidazol-2-yl)azepan-1-yl)propan-1-one,
2-(methylthio)-1-(2-(5-(p-tolyl)-1H-imidazol-2-yl)piperidin-1-yl)propan-1-one,
2-(methylthio)-1-(2-(5-(p-tolyl)-4H-1,2,4-triazol-3-yl)piperidin-1-yl)propan-1-one,
2-(methylthio)-1-(2-(5-(p-tolyl)isoxazol-3-yl)piperidin-1-yl)propan-1-one,
2-(methylthio)-1-(2-(5-(p-tolyl)oxazol-2-yl)piperidin-1-yl)propan-1-one,
2-(methylthio)-1-(2-(5-(p-tolyl)thiazol-2-yl)piperidin-1-yl)propan-1-one,
2-(methylthio)-1-(2-(5-phenyl-1H-imidazol-2-yl)piperidin-1-yl)propan-1-one,
2-(methylthio)-1-(2-(5-phenyl-1H-imidazol-2-yl)pyrrolidin-1-yl)propan-1-one,
2-(methylthio)-1-(2-(5-phenyl-4H-1,2,4-triazol-3-yl)piperidin-1-yl)propan-1-one,
2-(methylthio)-1-(2-(5-phenyloxazol-2-yl)piperidin-1-yl)propan-1-one,
2-(methylthio)-1-(4-(5-(p-tolyl)-1H-imidazol-2-yl)thiazolidin-3-yl)propan-1-one,
2,2,3-trimethyl-1-(2-(5-(p-tolyl)-1H-imidazol-2-yl)piperidin-1-yl)but-3-en-1-one,
2,2,3-trimethyl-1-(2-(5-(p-tolyl)-1H-imidazol-2-yl)piperidin-1-yl)butan-1-one,
2,2-dimethyl-1-(2-(5-(p-tolyl)-1H-imidazol-2-yl)piperidin-1-yl)but-3-en-1-one,
2,2-dimethyl-1-(2-(5-(p-tolyl)-1H-imidazol-2-yl)piperidin-1-yl)butan-1-one,
2,3-dimethoxy-1-(2-(4-phenyl-1H-imidazol-2-yl)piperidin-1-yl)propan-1-one,
2,3-dimethyl-1-(2-(4-phenyl-1H-imidazol-2-yl)piperidin-1-yl)butan-1-one,
2,5-difluoro-4-(2-(1-(2-(methylthio)propanoyl)piperidin-2-yl)-1H-imidazol-4-yl)benzonitrile,
2-amino-1-(2-(5-(p-tolyl)-1H-imidazol-2-yl)piperidin-1-yl)propan-1-one,
2-cyclobutyl-1-(2-(4-phenyl-1H-imidazol-2-yl)piperidin-1-yl)propan-1-one,
2-cyclopentyl-2-ethoxy-1-(2-(4-phenyl-1H-imidazol-2-yl)piperidin-1-yl)ethan-1-one,
2-cyclopropyl-1-(2-(4-phenyl-1H-imidazol-2-yl)piperidin-1-yl)ethan-1-one,
2-cyclopropyl-1-(2-(4-phenyl-1H-imidazol-2-yl)piperidin-1-yl)propan-1-one,
2-cyclopropyl-2-methoxy-1-(2-(4-phenyl-1H-imidazol-2-yl)piperidin-1-yl)ethan-1-one,
2-ethoxy-1-(2-(5-(3-fluorophenyl)-1H-imidazol-2-yl)piperidin-1-yl)propan-1-one,
2-ethyl-1-(2-(4-phenyl-1H-imidazol-2-yl)piperidin-1-yl)but-2-en-1-one,
2-ethyl-1-(2-(4-phenyl-1H-imidazol-2-yl)piperidin-1-yl)butan-1-one,
2-fluoro-4-(2-(1-(2-(methylthio)propanoyl)piperidin-2-yl)-1H-imidazol-5-yl)benzonitrile,
2-mercapto-1-(2-(5-(p-tolyl)-1H-imidazol-2-yl)piperidin-1-yl)propan-1-one,
2-methoxy-1-(2-(4-phenyl-1H-imidazol-2-yl)piperidin-1-yl)butan-1-one,
2-methoxy-1-(2-(4-phenyl-1H-imidazol-2-yl)piperidin-1-yl)propan-1-one,
2-methoxy-1-(2-(5-(p-tolyl)-1H-imidazol-2-yl)piperidin-1-yl)propan-1-one,
2-methyl-1-(2-(4-phenyl-1H-imidazol-2-yl)piperidin-1-yl)butan-1-one,
2-methyl-1-(2-(4-phenyl-1H-imidazol-2-yl)piperidin-1-yl)propan-1-one,
2-methyl-1-(2-(5-(p-tolyl)-1H-imidazol-2-yl)piperidin-1-yl)but-3-en-1-one,
2-methyl-1-(2-(5-(p-tolyl)-1H-imidazol-2-yl)piperidin-1-yl)butan-1-one,
2-methyl-1-(2-(5-(p-tolyl)-1H-imidazol-2-yl)piperidin-1-yl)butane-1,3-dione,
2-methyl-1-(2-(5-(p-tolyl)-1H-imidazol-2-yl)piperidin-1-yl)pentan-1-one,
2-methyl-1-(2-(5-(p-tolyl)-1H-imidazol-2-yl)piperidin-1-yl)prop-2-en-1-one,
2-methyl-1-(2-(5-(p-tolyl)-1H-imidazol-2-yl)piperidin-1-yl)propan-1-one,
2-methyl-2-(methylthio)-1-(2-(5-(p-tolyl)-1H-imidazol-2-yl)piperidin-1-yl)propan-1-one,
2-methyl-3-(methylthio)-1-(2-(4-phenyl-1H-imidazol-2-yl)piperidin-1-yl)propan-1-one,
2-methylene-1-(2-(5-(p-tolyl)-1H-imidazol-2-yl)piperidin-1-yl)butan-1-one,
3-(2-(1-(2-(methylthio)propanoyl)piperidin-2-yl)-1H-imidazol-4-yl)benzonitrile,
3,3,3-trifluoro-2-methyl-1-(2-(4-phenyl-1H-imidazol-2-yl)piperidin-1-yl)propan-1-one,
3,3,4,4,4-pentafluoro-1-(2-(4-phenyl-1H-imidazol-2-yl)piperidin-1-yl)butan-1-one,
3,3-difluoro-1-(2-(4-phenyl-1H-imidazol-2-yl)piperidin-1-yl)butan-1-one,
3-fluoro-4-(2-(1-(2-(methylthio)propanoyl)piperidin-2-yl)-1H-imidazol-5-yl)benzonitrile,
3-methyl-I-(2-(4-phenyl-1H-imidazol-2-yl)piperidin-1-yl)but-2-en-1-one,
3-methyl-1-(2-(4-phenyl-1H-imidazol-2-yl)piperidin-1-yl)butan-1-one,
4-(2-(1-(2-(methylthio)propanoyl)piperidin-2-yl)-1H-imidazol-4-yl)benzonitrile,
4-(2-(1-(2-(methylthio)propanoyl)piperidin-2-yl)-1H-imidazol-5-yl)benzaldehyde,
4,4,4trifluoro-2-methyl-1-(2-(4-phenyl-1H-imidazol-2-yl)piperidin-1-yl)butan-1-one,
bicyclo[1.1.1]pentan-1-yl(2-(4-phenyl-1H-imidazol-2-yl)piperidin-1-yl)methanone,
cyclobutyl(2-(5-(3-fluorophenyl)-1H-imidazol-2-yl)piperidin-1-yl)methanone,
cyclopentyl(2-(4-phenyl-1H-imidazol-2-yl)piperidin-1-yl)methanone,
cyclopropyl(2-(4-phenyl-1H-imidazol-2-yl)piperidin-1-yl)methanone.
methyl-4-(2-(1-(2-(methylthio)propanoyl)piperidin-2-yl)-1H-imidazol-5-yl)benzoate,
N-(2-hydroxyethyl)-4-(2-(1-(2-(methylthio)propanoyl)piperidin-2-yl)-1H-imidazol-4-yl)benzenesulfonamide,
2-(methylthio)-1-(2-(5-(3-nitrophenyl)-1H-imidazol-2-yl)piperidin-1-yl)propan-1-one,
N-(4-(2-(1-(2-(methylthio)propanoyl)piperidin-2-yl)-H-imidazol-4-yl)phenyl)methane-sulfonamide,
2-(methylthio)-1-(2-(5-(4-nitrophenyl)-1H-imidazol-2-yl)piperidin-1-yl)propan-1-one, (Compound A6),
1-(sec-butylsulfinyl)-2-(5-(p-tolyl)-1H-imidazol-2-yl)piperidine,
1-(see-butylsulfonyl)-2-(5-(p-tolyl)-1H-imidazol-2-yl)piperidine,
1-(isopropylsulfinyl)-2-(5-(p-tolyl)-1H-imidazol-2-yl)piperidine,
1-(isopropylsulfonyl)-2-(5-(p-tolyl)-1H-imidazol-2-yl)piperidine,
1-((1-(methylthio)ethyl)sulfinyl)-2-(5-(p-tolyl)-1H-imidazol-2-yl)piperidine,
1-((1-(methylthio)ethyl)sulfonyl)-2-(5-(p-tolyl)-1H-imidazol-2-yl)piperidine,
1-((2-(methylthio)propan-2-yl)sulfinyl)-2-(5-(p-tolyl)-1H-imidazol-2-yl)piperidine,
1-((2-(methylthio)propan-2-yl)sulfonyl)-2-(5-(p-tolyl)-1H-imidazol-2-yl)piperidine,
1-(but-3-en-2-ylsulfinyl)-2-(5-(p-tolyl)-1H-imidazol-2-yl)piperidine,
1-(but-3-en-2-ylsulfonyl)-2-(5-(p-tolyl)-1H-imidazol-2-yl)piperidine,
1-(pent-3-en-2-ylsulfinyl)-2-(5-(p-tolyl)-1H-imidazol-2-yl)piperidine,
1-(pent-3-en-2-ylsulfonyl)-2-(5-(p-tolyl)-1H-imidazol-2-yl)piperidine,
1-((2-methylbut-3-en-2-yl)sulfinyl)-2-(5-(p-tolyl)-1H-imidazol-2-yl)piperidine,
1-((2-methylbut-3-en-2-yl)sulfonyl)-2-(5-(p-tolyl)-1H-imidazol-2-yl)piperidine,
1-(2-(5-(4-(hydroxymethyl)-phenyl)-1H-imidazol-2-yl)piperidin-1-yl)-2-methylbutan-1-one,
2-(methylsulfonyl)-1-(2-(5-(p-tolyl)-1H-imidazol-2-yl)piperidin-1-yl)propan-1-one,
1-(4-hydroxy-2-(5-(p-tolyl)-1H-imidazol-2-yl)piperidin-1-yl)-2-(methylthio)propan-1-one,
1-(5-hydroxy-2-(5-(p-tolyl)-1H-imidazol-2-yl)piperidin-1-yl)-2-(methylthio)propan-1-one,
1-(4-hydroxy-2-(5-(p-tolyl)-1H-imidazol-2-yl)piperidin-1-yl)-2-methylbutan-1-one,
1-(5-hydroxy-2-(5-(p-tolyl)-1H-imidazol-2-yl)piperidin-1-yl)-2-methylbutan-1-one,
1-(2-(5-(4-(hydroxymethyl)phenyl)-1H-imidazol-2-yl)piperidin-1-yl)-2-(methylsulfonyl)pro-pan-1-one,
1-(4-hydroxy-2-(5-(p-tolyl)-1H-imidazol-2-yl)piperidin-1-yl)-2-(methylsulfonyl)propan-1-one,
1-(5-hydroxy-2-(5-(p-tolyl)-1H-imidazol-2-yl)piperidin-1-yl)-2-(methylsulfonyl)propan-1-one,
1-(2-hydroxy-6-(5-(p-tolyl)-1H-imidazol-2-yl)piperidin-1-yl)-2-(methylthio)propan-1-one,
1-(2-hydroxy-6-(5-(p-tolyl)-1H-imidazol-2-yl)piperidin-1-yl)-2-methylbutan-1-one,
1-(2-hydroxy-6-(5-(p-tolyl)-1H-imidazol-2-yl)piperidin-1-yl)-2-(methylsulfonyl)propan-1-one,
1-(3-hydroxy-2-(5-(p-tolyl)-1H-imidazol-2-yl)piperidin-1-yl)-2-(methylthio)propan-1-one,
1-(3-hydroxy-2-(5-(p-tolyl)-1H-imidazol-2-yl)piperidin-1-yl)-2-methylbutan-1-one,
1-(3-hydroxy-2-(5-(p-tolyl)-1H-imidazol-2-yl)piperidin-1-yl)-2-(methylsulfonyl)propan-1-one,
1-(2-(5-(4-(hydroxymethyl)phenyl)-1H-imidazol-2-yl)piperidin-1-yl)-2-(methylsulfinyl)-pro-pan-1-one,
1-(4-hydroxy-2-(5-(p-tolyl)-1H-imidazol-2-yl)piperidin-1-yl)-2-(methylsulfinyl)propan-1-one.
1-(5-hydroxy-2-(5-(p-tolyl)-1H-imidazol-2-yl)piperidin-1-yl)-2-(methylsulfinyl)propan-1-one,
1-(2-hydroxy-6-(5-(p-tolyl)-1H-imidazol-2-yl)piperidin-1-yl)-2-(methylsulfinyl)propan-1-one.
1-(3-hydroxy-2-(5-(p-tolyl)-1H-imidazol-2-yl)piperidin-1-yl)-2-(methylsulfinyl)propan-1-one,
1-(2-hydroxy-6-(5-(4-hydroxymethyl)phenyl)-1H-imidazol-2-yl)piperidin-1-yl)-2-(methyl-thio)propan-1-one,
1-(5-hydroxy-2-(5-(4-(hydroxymethyl)phenyl)-1H-imidazol-2-yl)piperidin-1-yl)-2-(methyl-thio)propan-1-one,
1-(4-hydroxy-2-(5-(4-(hydroxymethyl)phenyl)-1H-imidazol-2-yl)piperidin-1-yl)-2-(methyl-thio)propan-1-one,
1-(3-hydroxy-2-(5-(4-(hydroxymethyl)-phenyl)-1H-imidazol-2-yl)piperidin-1-yl)-2-(methyl-thio)propan-1-one,
1-(2-hydroxy-6-(5-(4-(hydroxymethyl)phenyl)-1H-imidazol-2-yl)piperidin-1-yl)-2-methyl-butan-1-one,
1-(5-hydroxy-2-(5-(4-hydroxymethyl)phenyl)-1H-imidazol-2-yl)piperidin-1-yl)-2-methyl-butan-1-one,
1-(4-hydroxy-2-(5-(4-(hydroxymethyl)phenyl)-1H-imidazol-2-yl)piperidin-1-yl)-2-methyl-butan-1-one,
1-(3-hydroxy-2-(5-(4-(hydroxymethyl)phenyl)-1H-imidazol-2-yl)piperidin-1-yl)-2-methyl-butan-1-one,
4-(2-(1-(2-methylbutanoyl)piperidin-2-yl)-1H-imidazol-5-yl)benzaldehyde,
4-(2-(1-(2-(methylthio)propanoyl)piperidin-2-yl)-1H-imidazol-5-yl)benzoic acid,
4-(2-(1-(2-methylbutanoyl)piperidin-2-yl)-1H-imidazol-5-yl)benzoic acid,
(2,2-dimethylcyclopropyl)(2-(5-(p-tolyl)-1H-imidazol-2-yl)piperidin-1-yl)methanone,
1-(2-(5-bromo-1H-imidazol-2-yl)piperidin-1-yl)-2-methylbutan-1-one,
1-(2-(5-(4-chlorophenyl)-1H-imidazol-2-yl)piperidin-1-yl)-2-methylbutan-1-one,
2-(4-(2-(1-(2-(methylthio)propanoyl)piperidin-2-yl)-1H-imidazol-5-yl)phenyl)acetonitrile,
2-(4-(2-(1-(2-methylbutanoyl)piperidin-2-yl)-1H-imidazol-5-yl)phenyl)acetonitrile,
2-(4-(2-(1-(2-methylbutanoyl)piperidin-2-yl)-1H-imidazol-5-yl)phenyl)acetonitrile,
2,3-dimethyl-1-(2-(5-(p-tolyl)-1H-imidazol-2-yl)piperidin-1-yl)butan-1-one,
2-methyl-1-(2-(5-(o-tolyl)-1H-imidazol-2-yl)piperidin-1-yl)butan-1-one,
2-methyl-1-(2-(5-(m-tolyl)-1H-imidazol-2-yl)piperidin-1-yl)butan-1-one,
1-(2-(5-(4-(hydroxymethyl)phenyl)-1H-imidazol-2-yl)piperidin-1-yl)-2-methylbutan-1-one,
1-(isopropylsulfonyl)-2-(5-(p-tolyl)-1H-imidazol-2-yl)piperidine,
1-(sec-butylsulfonyl)-2-(5-(p-tolyl)-1H-imidazol-2-yl)piperidine,
2-methyl-1-(2-(5-(5-methylthiophen-2-yl)-1H-imidazol-2-yl)piperidin-1-yl)butan-1-one,
2-(methylthio)-1-(2-(5-(p-tolyl)-1H-imidazol-2-yl)piperazin-1-yl)propan-1-one,
2-(methylthio)-1-(2-(4-(p-tolyl)oxazol-2-yl)piperidin-1-yl)propan-1-one,
2-methyl-1-(2-(4-(p-tolyl)oxazol-2-yl)piperidin-1-yl)butan-1-one
   and mixtures thereof

### Physiological Cooling Agents

Physiological cooling agents forming group (b1) are chemical substances initiating a cooling effect when brought into contact with human skin or mucosa. Typically, these agents represent menthol or menthol compounds are preferably selected from the group formed by the species depicted in the following table (including their optical isomers and racemates):

| Component | FEMA/ | | |
|---|---|---|---|
| Menthol | GRAS | Trademark | Supplier |
| Menthol glyceryl acetal | 3807 | Frescolatº | Symrise |
| | | MGA | AG |
| Menthol glyceryl ketal | 3808 | Frescolatº | Symrise |
| Menthol menthyl ether | | MGA | AG |
| Menthol glyceryl acetal | | | |
| Menthone glyceryl ketal | | | |
| Menthoxy-1,2-propandiol | 3784 | | |
| Menthoxy-2-methyl-1,2-propanediol | 3849 | | |
| Menthyl acetate | | SymFreshº | Symrise |
| | | RF | AG |
| Menthyl ethylene glycol carbonate | 3805 | Frescolatº | Symrise |
| Menthyl formiate | | MGC | AG |
| Menthyl glutamate | 4006 | | |
| Menthyl glycerol carbonate | | | |
| Menthyl hydroxy isobutyrate | | | |
| Menthyl isobutyrate | | | |
| Menthyl lactate | | Frescolatº | Symrise |
| Menthyl malonate | | ML | AG |
| Menthyl methyl ether | | | |
| Menthyl N-ethyl oxamate | | | |
| Menthyl propylene glycol carbonate | 3806 | Frescolatº | Symrise |
| Menthyl pyroglutamate | | MPC | AG |
| Menthyl-(2-methoxy)acetate | | | |
| Menthyl-(2-methoxyethoxy)acetate | | | |
| Menthyl succinate | 3810 | | |
| O-Menthyl succinic acid ester amide | | | |
| O-Menthyl succinic acid ester-NN- | | | |
| (dimethyl)amide | | | |
| Menthane carboxylic acid-N-(4- | | | |
| cyanophenyl)amide | | | |
| Menthane carboxylic acid-N-(4- | | | |
| cyanomethylphenyl)amide | | | |
| Menthane carboxylic acid- | | | |
| N-ethylamide | | | |
| (WS-3) | | | |
| (WS-4) | | | |
| Nº-(menthane carbonyl) glycinc | | | |
| ethylester | | | |
| (WS-5) | | | |
| (1R,2S,5R)-N-(4-Methoxyphenyl)- | | | |
| 5-methyl- | | | |
| 2-(1-isopropyl)cyclohexane- | | | |
| carboxamide | | | |
| (WS-12) | | | |
| (WS-14) | | | |
| 2,3-dimethyl-2-(2-propyl)-butyric acid- | | | |
| N-methylamide (WS23) | | | |
| Isopulegol acetate | | | |
| p-Menthane-3,8-diol | | | |
| Cubebol | | | |
| 3-Methyl-2(1-pyrrolidinyl)-2- | | | |
| cyclopentene-1-one) | | | |
| Tetrahydropyrimidine-2-one | | | |
| N-(2-(pyridin-2-yl)ethyl)-3-p- | | X Cool | Symrise |
| menthanecarboxamide | | | AG |
| [(1R,2S,5R)-2-isopropyl-5-methyl- | | | |
| cyclohexyl] 2-(ethylamino)-2- | | | |
| oxo-acetate | | | |

### Menthol Derivatives

A first important representative of the substances forming component (b) is mono-menthyl succinate, which as a substance was patented as early as 1963 by Brown & Williamson Tobacco Corp. (U.S. Pat. No. 3,111,127) and as a refrigerant is the subject of property rights U.S. Pat. Nos. 5,725,865 and 5,843,466 (V. Mane Fils). Both the succinate and the analogous monomenthyl glutarate are important representatives of monomenthyl esters based on di- and polycarboxylic acids:

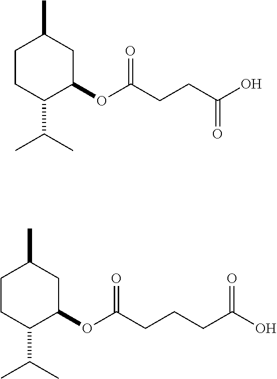 [SMILES: CC(C)[C@@H]1CC[C@@H](C)C[C@H]1OC(=O)CCC(=O)O ; CC(C)[C@@H]1CC[C@@H](C)C[C@H]1OC(=O)CCCC(=O)O]

Examples of applications of these substances can be found, for example, in the printed documents WO 2003 043431 (Unilever) or EP 1332772 A1 (IFF).

The next important group of menthol compounds preferred in the sense of the invention comprises carbonate esters of menthol and polyols, such as glycols, glycerol or carbohydrates, such as menthol ethylene glycol carbonates, menthol propylene glycol carbonates, menthol 2-methyl-1,2-propanediol carbonates or the corresponding sugar derivatives:

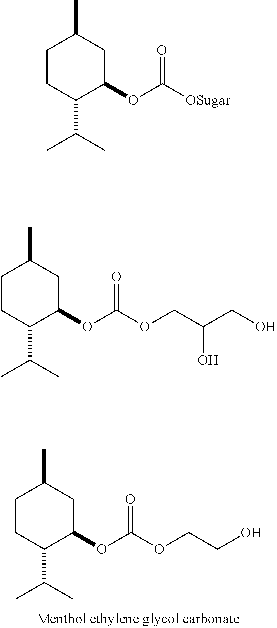

The use of such substances as a cooling agent for cigarettes is, for example, the subject of the 1968 publication U.S. Pat. No. 3,419,543 (Mold et al.); their use as a physiological cooling agent is claimed in DE 4226043 A1 (H&R).

In the sense of the invention, the menthol compounds menthyl lactate) and, in particular, menthone glyceryl acetal or menthone glyceryl ketal are preferred.

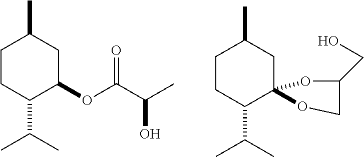

The former structure is obtained by esterification of lactic acid with menthol, the latter by acetylation of menthone with glycerol (cf. DE 2608226 A1, H&R). This group of compounds also includes 3-(1-menthoxy)-1,2,propanediol, also known as Cooling Agent 10 (U.S. Pat. No. 6,328,982, TIC), and 3-(1-menthoxy)-2-methyl-1,2,propanediol, which has an additional methyl group.

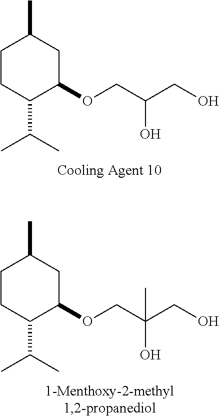 [SMILES: CC(C)[C@@H]1CC[C@@H](C)C[C@H]1OCC(O)CO ; CC(C)[C@@H]1CC[C@@H](C)C[C@H]1OCC(C)(O)CO]

For example, 3-(1-menthoxy)-1,2,propanediol is prepared starting from menthol according to the following scheme (see U.S. Pat. No. 4,459,425, Takasago):

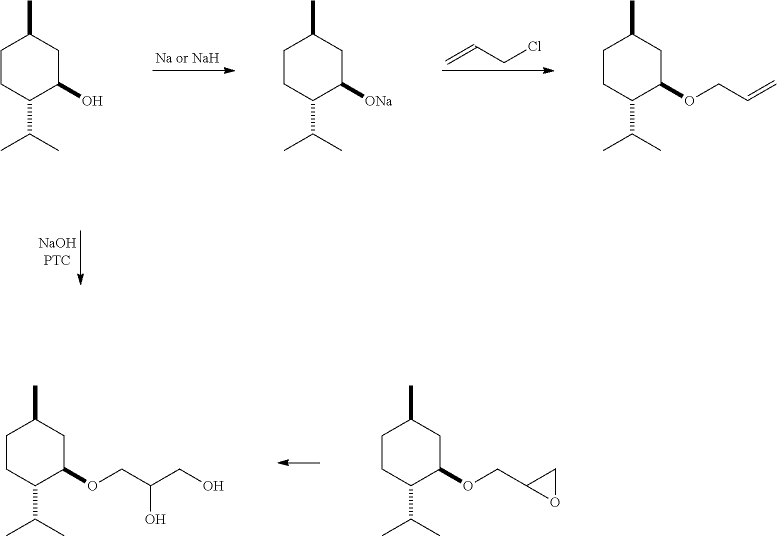

Alternative routes in which menthol is reacted with epichlorohydrin in the first step are described in U.S. Pat. Nos. 6,407,293 and 6,515,188 (Takasago). The following is an overview of preferred menthol compounds characterized by CO bonding:

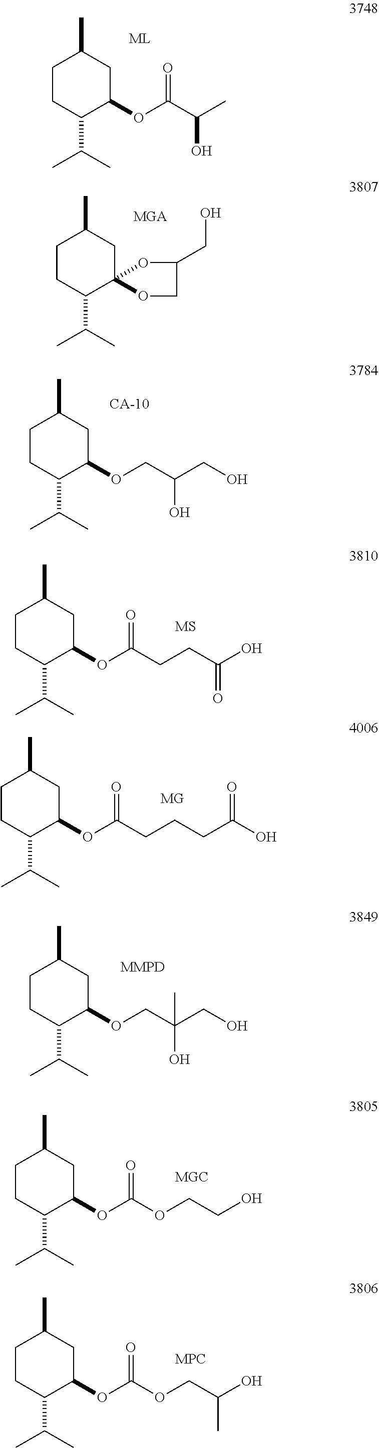

Among these substances, menthone glyceryl acetal/ketal and menthyl lactate as well as menthol ethylene glycol carbonate and menthol propylene glycol carbonate.

### WS Cooling Agents

In the 1970s, menthol compounds were developed for the first time which have a C—C bond in the 3-position and of which a number of representatives can also be used in the sense of the invention. These substances are generally referred to as WS types. The basic body is a menthol derivative in which the hydroxyl group is replaced by a carboxyl group (WS-1). All other WS types are derived from this structure, such as the species WS-3, WS-4, WS-5, WS-12, WS-14 and WS-30, which are also preferred in the sense of the invention. The following two diagrams show the synthesis routes:

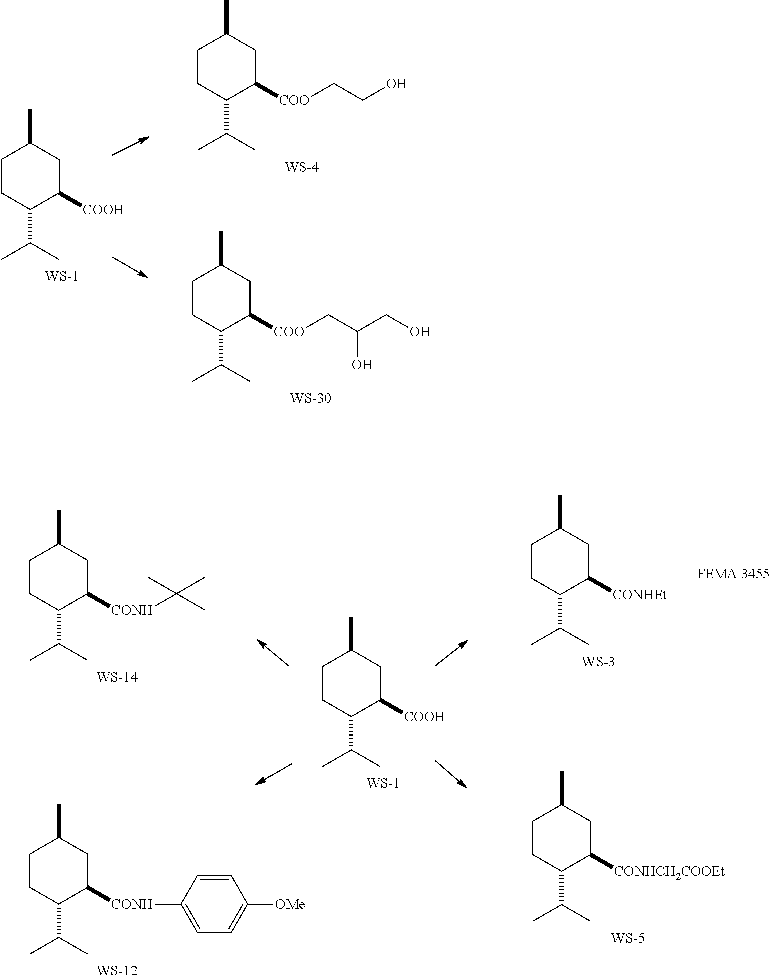

The esters derived from WS-1 are described, for example, in U.S. Pat. No. 4,157,384, and the corresponding N-substituted amides in J. Soc. Cosmet. Chem. pp. 185-200 (1978).

Physiological cooling agents which are particularly preferred encompass the following species:
Menthol
Menthol glyceryl acetal
Menthol glyceryl ketal
Menthol menthyl ether
Menthone glyceryl acetal
Menthone glyceryl ketal
Menthoxy-1,2-propandiol
Menthoxy-2-methyl-1,2-propanediol
Menthyl acetate
Menthyl ethylene glycol carbonate
Menthyl formiate
Menthyl glutamate
Menthyl glycerol carbonate
Menthyl hydroxy isobutyrate
Menthyl isobutyrate
Menthyl lactate
Menthyl malonate
Menthyl methyl ether
Menthyl N-ethyl oxamate
Menthyl propylene glycol carbonate
Menthyl pyroglutamate
Menthyl-(2-methoxy)acetate
Menthyl-(2-methoxyethoxy)acetate
Menthyl succinate
O-Menthyl succinic acid ester amide
O-Menthyl succinic acid ester-NN-(dimethyl)amide
Menthane carboxylic acid-N-(4-cyanophenyl)amide
Menthane carboxylic acid-N-(4-cyanomethylphenyl)amide
Menthane carboxylic acid-N-ethylamide (WS-3)
(WS-4)
N^{α}-(menthane carbonyl) glycine ethylester (WS-5)
(1R,2S,5R)—N-(4-Methoxyphenyl)-5-methyl-2-(1-isopropyl)cyclohexane-carboxamide (WS-12)
(WS-14)
2,3-dimethyl-2-(2-propyl)-butyric acid-N-methylamide (WS23)
Isopulegol acetate
p-Menthane-3,8-diol
Cubebol
3-Methyl-2(1-pyrrolidinyl)-2-cyclopentene-1-one)
Tetrahydropyrimidine-2-one
N-(2-(pyridin-2-yl)ethyl)-3-p-menthanecarboxamide
[(1R,2S,5R)-2-isopropyl-5-methyl-cyclohexyl] 2-(ethylamino)-2-oxo-acetate

### Physiological Warming Agents

Physiological warming agents forming group (b2) are chemical substances initiating a warming effect when brought into contact with human skin or mucosa. Suitable warming agents encompass the following species:
vanillyl derivatives, preferably vanillyl ethers
capsaicin;
allyl isothiocyanate;
gingerol;
4-(1-menthoxymethyl)-2-phenyl-1;3-dioxolan;
4-(1-menthoxymethyl)-2-(3′;4′-dihydroxyphenyl)-1;3-dioxolan;
4-(1-menthoxymethyl)-2-(2′-hydroxy-3′-methoxyphenyl)-3-dioxolan;
4-(1-menthoxymethyl)-2-(4′-methoxyphenyl)-3-dioxolan;
4-(1-menthoxymethyl)-2-(3′;4′methylenedioxyphenyl)-3-dioxolan;
4-(1-menthoxymethyl)-2-(3′-methoxy-4′-hydroxyphenyl)-3-dioxolan;
red pepper oil;
red pepper oleoresin;
ginger oleoresin;
nonylic acid vanillyl amide;
jambu oleoresin;
Zanthoxylum pieritum extract;
sanshool I;
sanshool II;
sanshoamide;
black pepper extract;
chavicine;
piperine;
spilanthol;
the modulators or warming agents disclosed in U.S. Pat. No. 6,780,443 (as far as the nature of the modulators are concerned this document is hereby incorporated by reference)
   and mixtures thereof.

In a preferred embodiment said warming agent represent a vanillyl ether according to formula (I)

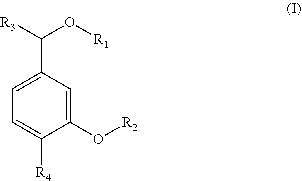

wherein R¹ stands for hydrogen, or a C₁-C₇ alkyl group; R² stands for a C₁-C₃ alkyl radical, R³ stands for hydrogen or a C₃-C₉ alkoxyl group; R⁴ stands for hydroxyl or a —OC(O)CH₃ group; and wherein R² and R³ can be covalent bounded to form a cyclic acetal; said acetal optionally substituted by a C₂-C₈ alkyl group.

In a particularly preferred embodiment said warming agents represent a vanillyl ether according to formula (II)

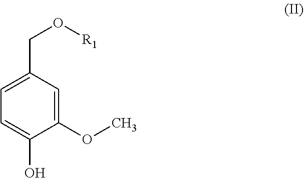

wherein R¹ stand for hydrogen or a C₁-C₇ alkyl group.

Most preferred said warming agents represent a vanillyl ether according to formula (III)

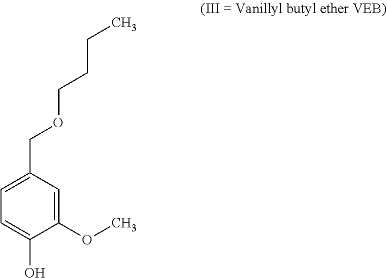

### Aroma or Flavoring Compounds

Aroma compounds and flavouring agents forming group (c) are well known in the art and can be chosen from synthetic flavouring liquid and/or oils derived from plants leaves, flowers, fruits and so forth, and combinations thereof. Representative flavouring liquids include: artificial, natural or synthetic fruit flavours such as eucalyptus, lemon, orange, banana, grape, lime, apricot and grapefruit oils and fruit essences including apple, strawberry, cherry, orange, pineapple and so forth; bean and nut derived flavours such as coffee, cocoa, cola, peanut, almond and so forth; and root derived flavours such as licorice or ginger.

Suitable aroma or flavouring compounds encompass the following species:
12-methyltridecanal;
acetaldehyde;
acetanisole;
acethylthiazole-2;
acethylthiazoline-2;
acetic acid;
acetyl methylcarbinol;
acetyl pyrazine-2;
acetyl pyridine-2;
allyl capronate;
allyl isothiocyanate;
amyl cinnamaldehyde-alpha;
anethole;
anisaldehyde-para;
anisic alcohol;
benzaldehyde;
benzyl acetate;
borneol-1;
butyl acetate;
butylidene phthalide-3;
butyric acid;
camphor;
caproic acid;
carvacrol;
carvomenthone;
carvon-d;
carvon-l;
carvylacetate-cis;
caryophyllene;
cineole-1;4;
cineole-1;8;
cinnamalcohol;
cinnamaldehyde;
cinnamyl acetate;
citral;
citronellal;
citronellol;
citronellyl acetate;
cresol
cuminaldehyde;
cyclopentadecanolide;
damascenone-alpha;
damascenone-beta;
damascone-alpha;
damascoe-beta;
decadienal-2;4;
decadienal-2;4;
decalactone delta;
decalacton gamma;
decanal;
dehydromenthofurolactone;
diallyl disulfide;
diethylpyrazine-2;3;
dihydroanethol;
dihydrocarvone;
dihydrocoumarin;
dihydroionone-beta;
dihydromenthofurolactone;
dihydronootkatone;
dimethyl lanthranilate;
dimethyl isopropyldithiazine;
dimethyl pyrazine;
dimethyl sulfide;
diphenyloxide;
divanilline;
d-menthol;
d-menthone;
d-menthyl acetate;
dodecalactone-delta;
dodecalactone-gamma;
epithiomenthan-1;8;
ethyl acetate;
ethyl butyrate;
ethyl capronate;
ethyl caprylate;
ethyl cinnamate;
ethyl isobutyrate;
ethyl isovaleria-nate;
ethyl lactate;
ethyl maltol;
ethyl methyl butyrate-2;
ethyl methyl thiopropionate;
ethyl phenol-4;
ethyl vanillin;
eugenol;
fenchol;
filbertone;
frambinone methyl ether;
frambinone;
furaneol;
furfurylthiol;
geraniol;
geranyl acetate;
geranyl isobutyrate;
guaiacol;
heliotropin;
heptalactone-gamma;
heptanone-2;
heptenal-Z-4;
hexalactone-gamma;
hexanal
hexanol;
hexenol-E-2;
hexenol-Z-3;
hexenyl acetate-E-2;
hexenyl acetate-Z-3;
hexenyl capronate-Z-3;
hexyl acetate;
hexyl cinnamaldehyde-alpha;
hotrienol;
indole;
ionon-beta;
ionone-alpha;
isoamyl acetate;
isoamyl butyrate;
isoamyl isovaerianate;
isobutyl acetate;
isobutyl thiazole;
isobutylmethoxypyrazine
isobutyraldehyde;
isoeugenol;
Isomenthon;
isopropylmethoxypyrazine;
isopropylmethylpyrazine-2;4;
isopulegol;
isova-leraldehyde;
jasmine lactone;
jasmon cis;
ketoisophorone;
limonene-d;
linalool oxide;
linalool-d;
linalool-l;
linalyl acetate;
l-menthol;
l-menthone;
l-menthyl acetate;
maltol;
massoilactone;
melonal;
menthadienyl acetate;
menthofurolactone;
menththiol-1;8;
methional;
methoxymethylpyrazine-2;3;
methyl anthranilate;
methyl butyl acetate-2;
methyl butyrate;
methyl cinnamate;
methyl dihydrojasmonate;
methyl furanthiol-2;3;
methyl jasmonate;
methyl salicylate;
methyl tetrahydrofuranthiol-2;3;
methyl thiobutyrate;
methyl thiohexyl acetate-1;3;
methylcyclopen-tenolone;
methylthiohexanol-1;3;
myrtenal;
naringin;
neo-menthol;
neral;
nerol;
nerylacetate;
nonadienal-2;4;
nonadienal-2;6;
nonalactone delta;
nonalactone gamma;
nonenal-E-2;
nonenal-Z-6;
nonenol-Z-6;
nootkatone;
octalactone gamma;
octalactone delta;
octanal
octenol-1;3;
pellitorin;
pentenone-1;3;
pentyl acetate;
phenyl ethyl acetate;
phenyl ethyl alcohol;
phenyl acetaldehyde;
pinene-alpha;
pinene-beta;
piperitanate;
piperitone
prenyl thioacetate;
prenyl thiol;
reubenamine;
rose oxide;
rubescenamine;
sabinene hydrate;
scatole;
sotolone;
styrolyl acetate;
terpinenol-4;
terpineol;
thiohexanol-1;3;
thiohexyl acetate-1;3;
thiomenthanone-8;3;
thiopentanone-4;4;2;
thymol;
tridecatrienal;
trimethyl pyrazine;
undecadienal-2;4;
undecalactone-gamma and delta;
undecatriene;
vanillin;
vinyl guaiacol;
whisky lactone;
   and mixtures thereof.

In particular preferred aroma or flavouring compounds encompass menthol, cineol, eugenol, thymol, cinnamic aldehyde, peppermint oil, spearmint oil, eucalyptus oil, thyme oil, cinnamon oil, clove oil, spruce needle oil, fennel oil, sage oil, aniseed oil, star anise oil, chamomile oil, and caraway oil, and their mixtures.

### Fragrances

The fragrances forming group (d) can be used as single components or in the form of more or less complex mixtures. The species may be obtained from natural sources or prepared by organic synthesis.

### Natural Perfumes

Natural perfumes include the extracts of blossoms (lily, lavender, rose, jasmine, neroli, ylangylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used.

Typically, the synthetic fragrances represent aldehydes, ketones, alcohols, ethers, esters, hydrocarbons their mixtures. In the following these types of fragrances are illustrated but not limited by examples:

### Aldehydes

Examples for suitable fragrances showing an aldehyde structure encompass melonal, triplal, ligustral, adoxal, anisaldehyde, cymal, ethylvanillin, florhydral, floralozon, helional, heliotropin, hydroxycitronellal, koavon, laurinaldehyde, canthoxal, lyral, lilial, adoxal, anisaldehyde, cumal, methylnonyl-acetaldehyde, citronellal, citronellyloxy-acetaldehyde, cyclamenaldehyde, bourgeonal, p-tert.-bucinal, phenylacetaldehyde, undecylenaldehyde, vanillin; 2,6,10-trimethyl-9-undecenal, 3-dodecen-1-al, α-n-Amylzimtaldehyde, 4-methoxy-benz-aldehyde, benzaldehyde, 3-(4-tert-butylphenyl)-propanal,2-methyl-3-(para-methoxy-phe-nylpropanal), 2-methyl-4-(2,6,6-trimethyl-2(1)-cyclohexen-1-yl)butana1, 3-phenyl-2-pro-penal, cis-/trans-3,7-dimethyl-2,6-octadien-1-al, 3,7-dimethyl-6-octen-1-al,[(3,7-dimethyl-6-octenyl)-xy]-cetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxyaldehyde, 2-methyl-3-(isopropyl-phenyl)propanal, decyl aldehyde, 2,6-dimethyl-5-heptenal; 4-(tricyclo[5.2.1.0 (2,6)]-decylidene-8)butanal; octahydro-4,7-methano-1H-indenecarboxaldehyde; 3-ethoxy-4-hydroxybenzaldehyde, para-ethyl-alpha,alpha-dimethylhydrozimtaldehyde, α-methyl-3,4-(methylenedioxy)-hydrocinnamaldehyde, 3,4-methylenedioxybenzaldehyde, α-n-hexyl-cinnamaldehyde, m-cymene-7-carboxaldehyde, α-methylphenylacetaldehyde, 7-hydroxy-3,7-dimethyl octanal, undecenal, 2,4,6-trimethyl-3-cyclohexene-1-carboxaldehyde,4-(3)(4-methyl-3-pentenyl)-3-cyclohexen-carboxaldehyde, 1-dodecanal, 2,4-dimethyl-cyclohexene-3-carboxaldehyde,4-(4-hydroxy-4-methylpentyl)-3-cylohexene-1-carboxal-dehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, 3-(4-ethylphenyl)-2,2-dimethylpropanal, 3-(4-methoxyphenyl)-2-methylpropanal, methylno-nylacetaldehyde, 2-phenylpropan-1-al, 3-phenylprop-2-en-1-al, 3-phenyl-2-pentylprop-2-en-1-al, 3-phenyl-2-hexylprop-2-enal, 3-(4-isopropylphenyl)-2-methylpropan-1-al, 3-(4-ethylphenyl)-2,2-dimethylpropan-1-al, 3-(4-tert-butylphenyl)-2-methyl-propanal, 3-(3,4-Methylendioxy-phenyl)-2-methylpropan-1-al,3-(4-Ethylphenyl)-2,2-dimethylpropanal, 3-(3-Isopropylphenyl)-butan-1-al, 2,6-Dimethylhept-5-en-1-al,Dihydrozimtaldehyde, 1-methyl-4-(4-methyl-3-pentenyl)-3-cyclohexene-1-carboxaldehyde, 5- or 6-Methoxyhexahydro-4,7-methanoindan-1 or 2-carboxyaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexene-carboxyaldehyde, 7-hydroxy-3,7-dimethyl-octanal; trans-4-decenal, 2,6-nonadienal, p-tolylacetaldehyde; 4-methylphenylacetaldehyde, 2-methyl-4-(2,6,6-trimethyl-1-cyclohexen-1-yl)-2-butenal, o-methoxyzimtaldehyde, 3,5,6-trimethyl-3-cyclohexenecarboxaldehyde, 3,7-dimethyl-2-methylene-6-octenal, phenoxyacetaldehyde; 5,9-dimethyl-4,8-decadienal, peony aldehyde (6,10-dimethyl-3-oxa-5,9-undecadien-1-al), hexahydro-4,7-methanoindan-1-carboxaldehyde, octanal, 2-methyl octanal, alpha-methyl-4-(I-methylethyl)benzene-acetaldehyde, 6,6-dimethyl-2-norpinene-2-propionaldehyde, p-methyl phenoxy acetaldehyde, 2-methyl-3-phenyl-2-propen-1-al, 3,5,5-trimethylhexanal, hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral or its mixtures, lilial citral, 1-decanal, n-undecanal, n-dodecanal, hlorhydral, 2,4-dimethyl-3-cyclohexen-1-carboxaldehyde 4-methoxybenzaldehyde, 3-methoxy-4-hydroxy-benzalde-hyde, 3-ethoxy-4-hydroxybenzaldehyde, 3,4-methylendioxybenzaldehyde, 3,4-dimethoxybenzaldehyde and their mixtures.

### Ketones

Examples for suitable fragrances showing a ketone structure encompass buccoxime, iso jasmone, methyl beta naphthyl ketone, moschus indanone, tonalid/moschus plus, α-damascone, β-damascon, δ-damascone, Iso-damascone, damascenone, damarose, methyl-dihydrojasmonate, menthone, carvone, campher, fenchone, alphalonen, β-iononw, dihydro-β-Ionone, γ-methylionone, fleuramone, dihydrojasmone, cis-Jasmon, iso-E-Super, methyl cedrenylk etone, or methyl cedrylon, acetophenone, methyl aceto phenone, p-methoxyacetophenone, methyl-β-naphtyl ketone, benzylacetone, benzophenone, p-hydroxy phenylbutanone, celery Ketone or livescon, 6-osopropyl-deca-hydro-2-naphtone, dimethyloctenone, freskomenth, 4-(I-ethoxyvinyl)-3,3,5,5,-tetramethyly cyclohexanone, methylheptenone, 2-(2-(4-Methyl-3-cyclohexen-1-yl)propyl)-cyclopentanone, 1-(p-men-thene-6(2)-yl)-1-propanone,4-(4-Hydroxy-3-methoxyphenyl)-2-butanone, 2-Acetyl-3,3-di-methyl-norbornan, 6,7-dihydro-1,1,2,3,3-pentamethyl-4(5H)-indanone, 4-damascol, dulcinyl or cassione, gelsone, hexalone, isocyclemone E, Methylcyclocitrone, methyl lavender ketone, orivone, p-tert-butyl cyclohexanone, verdone, delphone, muscone, neobutenone, plicatone, veloutone, 2,4,4,7-tetramethyl-oct-6-en-3-one, tetrameran, hedion and their mixtures. The preferred ketones are selected from the group comprising adamascone, δ-damascone, iso-damascone, carvone, γ-methyl ionone, Iso-E-Super, 2,4,4,7-tetramethyloct-6-en-3-one, benzylacetone, β-damascone, damascenone, methyl dihydrojasmonate, methyl cedrylone, hedione and their mixtures

As explained above, said ketones or said aldehydes may show an aliphatic, cycloaliphatic, aromatic, ethylenically unsaturated structure or a mixture of these elements. The components may also include heteroatoms or show a polycyclic structure. Suitable substituents for all these structures are hydroxyl and/or amino groups. Further fragrances are compiled in the following document: Steffen Arctander, Published 1960 and 1969 respectively, Reprinted 2000 ISBN: Aroma Chemicals Vol. 1: 0-931710-37-5, Aroma Chemicals Vol. 2: 0-931710-38-3”, which is hereby incorporated by reference.

### Alcohols

Suitable fragrance alcohols encompass for example 10-undecen-1-ol, 2,6-dimethylheptan-2-ol, 2-methylbutanol, 2-methylpentanol,2-phenoxyethanol, 2-phenylpropanol, 2-tert-Butycyclohexanol, 3,5,5-trimethylcyclohexanol, 3-hexanol, 3-methyl-5-phenylpentanol, 3-octanol, 1-octen-3-ol, 3-phenylpropanol,4-heptenol, 4-isopropylcyclohexanol, 4-tert-butycyclohexanol, 6,8-dimethyl-2-nonanol,6-nonen-1-ol, 9-decen-1-ol, α-methyl benzylalcohol, α-terpineol, amylsalicylat, benzyl alcohol, benzyl salicylate, β-terpineol, butyl salicylate, citronellol, cyclohexyl salicylate, decanol, dihydromyrcenol, dimethyl benzylcarbinol, dimethyl heptanol, dimethyl octanol, ethyl salicylate, ethyl vanilin, anethol, eugenol, geraniol, heptanol, hexyl salicylat, isoborneol, isoeugenol, isopulegol, linalool, menthol, myrtenol, n-hexanol, nerol, nonanol, octanol, para-menthan-7-ol, phenylethylalkohol, phenol, phenyl salicylat, tetrahydro geraniol, tetrahydro linalool, thymol, trans-2-cis-6-nonadienol, trans-2-nonen-1-ol, trans-2-octenol, undecanol, vanillin, cinnamalcohol and their mixtures.

### Esters

Examples for suitable fragrances showing a ketone structure encompass benzyl acetate, phenoxyisobutyrate, p-tert.-butylcyclohexylacetate, linalylacetate, dimethylbenzylcarbinylacetate (DMBCA), phenylethylacetate, benzylacetate, ethylmethylphenylglycinate, allylcyclohexylpropionate, styrallylpropionate, benzylsalicylate, cyclohexylsalicylate, floramat, melusat, jasmacyclatat and their mixtures.

### Ethers

Examples for suitable fragrances showing a ketone structure encompass benzylethyl ether or ambroxan.

### Hydrocarbons

Examples for suitable fragrances representing hydrocarbons encompass terpenes, e.g. limonene and pinen.

Fragrances which are particularly preferred encompass the following species:
[(3,7-dimethyl-6-octenyl)-xy]-cetaldehyde,
1-(p-menthene-6(2)-yl)-1-propanone,
1,2,3,4,5,6,7,8-octahydro-8,8-dimethyl-2-naphthaldehyde,
10-undecen-1-al,
10-undecen-1-ol,
1-decanal,
1-dodecanal,
1-methyl-3-(4-methylpentyl)-3-cyclohexene-carboxyaldehyde,
1-methyl-4-(4-methyl-3-pentenyl)-3-cyclohexene-1-carboxaldehyde,
1-nonanal,
1-octanal,
1-octen-3-ol,
1-p-menthene-q-carboxaldehyde,
1-undecanal,
2-(2-(4-Methyl-3-cyclohexen-1-yl)propyl)-cyclopentanone,
2,4,4,7-tetramethyl-oct-6-en-3-one,
2,4,6-trimethyl-3-cyclohexene-1-carboxalde-hyde,
2,4-dimethyl-3-cyclohexen-1-carboxaldehyde 4-methoxybenzaldehyde,
2,4-dimethyl-3-cyclohexen-1-carboxyaldehyde,
2,6,10-trimethyl-5,9-undecadienal,
2,6,10-trimethyl-9-undecenal, 3-dodecen-1-al,
2,6-dimethyl-5-heptenal;
2,6-dimethylhept-5-en-1-al,
2,6-dimethylheptan-2-ol,
2,6-nonadienal,
2-acetyl-3,3-di-methyl-norbornan,
2-methyl decanal,
2-methyl octanal,
2-methyl undecanal,
2-methyl-3-(4-tertbutyl)propanal,
2-methyl-3-(isopropyl-phenyl)propanal,
2-methyl-3-(para-methoxy-phenylpropanal),
2-methyl-3-phenyl-2-propen-1-al,
2-methyl-4-(2,6,6-trimethyl-1-cyclohexen-1-yl)-2-butenal,
2-methylbutanol,
2-methylpentanol,
2-phenoxyethanol,
2-phenylpropan-1-al,
2-phenylpropanol,
2-tert-butycyclohexanol,
3-(3,4-methylenedioxy-phenyl)-2-methylpropan-1-al,
3-(3-isopropylphenyl)-butan-1-al,
3-(4-ethylphenyl)-2,2-dimethylpropan-1-al,
3-(4-ethylphenyl)-2,2-dimethylpropanal,
3-(4-isopropylphenyl)-2-methylpropan-1-al,
3-(4-methoxyphenyl)-2-methylpropanal,
3-(4-tert-butylphenyl)-2-methyl-propanal,
3-(4-tert-butylphenyl)-propanal,
3,4-dimethoxybenzaldehyde,
3,4-methylendioxybenzaldehyde,
3,5,5-trimethylcyclohexanol,
3,5,5-trimethylhexanal,
3,5,6-trimethyl-3-cyclohexenecarboxaldehyde,
3,7-dimethyl-2-methylene-6-octenal,
3,7-dimethyl-6-octen-1-al,
3,7-dimethyloctan-1-al,
3-ethoxy-4-hydroxybenzaldehyde,
3-hexanol,
3-methoxy-4-hydroxy-benzaldehyde,
3-methyl-5-phenyl-1-pentanal,
3-methyl-5-phenylpentanol,
3-octanol,
3-phenyl-2-hexylprop-2-enal,
3-phenyl-2-pentylprop-2-en-1-al,
3-phenyl-2-propenal,
3-phenylprop-2-en-1-al,
3-phenylpropanol,
3-propyl-bicyclo[2.2.1]-hept-5-ene-2-carbaldehyde,
4-(1-ethoxyvinyl)-3,3,5,5-tetramethyl cyclohexanone,
4-(3)(4-methyl-3-pentenyl)-3-cyclohexen-carboxaldehyde,
4-(4-hydroxy-3-methoxyphenyl)-2-butanone,
4-(4-hydroxy-4-methylpentyl)-3-cylohexene-1-carboxal-dehyde,
4-(tricyclo[5.2.1.0(2,6)]-decylidene-8)-butanal;
4-damascol,
4-heptenol,
4-hydroxy acetophenone
4-hydroxy-3-methoxybenzaldehyde,
4-isopropyl benzaldehyde,
4-isopropyl cyclohexanol,
4-methoxy benzaldehyde,
4-methylphenyl acetaldehyde,
4-tert-butyl cyclohexanol,
5,9-dimethyl-4,8-decadienal,
5-methoxyhexahydro-4,7-methanoindan-1-carboxyaldehyde,
5-methoxyhexahydro-4,7-methanoindan-2-carboxyaldehyde,
6,10-dimethyl-3-oxa-5,9-undecadien-1-al,
6,6-dimethyl-2-norpinene-2-propionaldehyde,
6,7-dihydro-1,1,2,3,3-pentamethyl-4(5H)-indanone,
6,8-dimethyl-2-nonanol,6-nonen-1-ol,
6-isopropyl-deca-hydro-2-naphtone,
6-methoxyhexahydro-4,7-methanoindan-1-carboxyaldehyde,
6-methoxyhexahydro-4,7-methanoindan-2-carboxyaldehyde,
7-hydroxy-3,7-dimethyl octanal,
7-methoxy-3,7-dimethyloctan-1-al,
9-decen-1-ol,
9-decenal,
acetophenone,
adoxal,
allyl cyclohexylpropionate,
alphalonen,
ambroxan,
amylsalicylat,
anethol,
anisaldehyde,
benzaldehyde,
benzophenone,
benzyl acetate,
benzyl acetone,
benzyl alcohol,
benzyl salicylate,
benzylethyl ether,
bourgeonal,
buccoxime,
butyl lactate,
butyl salicylate,
campher,
canthoxal,
carvone,
cassione,
celery cetone
chlorhydral,
cinnamalcohol.
cis-/trans-3,7-dimethyl-2,6-octadien-1-al,
cis-jasmon,
citral or its mixtures,
citronellal,
citronellol,
citronellyl oxyacetaldehyde,
cumal,
cyclamenaldehyde,
cyclohexyl salicylate,
cymal,
damarose,
damascenone,
decanol,
decyl aldehyde,
delphone,
dihydrojasmone,
dihydromyrcenol,
dihydrozimtaldehyde,
dihydro-β-lonone,
dimethyl benzylcarbinol,
dimethyl benzylcarbinylacetate,
dimethyl heptanol,
dimethyl octanol,
dimethyl octenone,
dulcinyl
ethyl salicylate,
ethyl vanilin,
ethylmethylphenyl glycinate,
eugenol,
fenchone,
fleuramone,
floralozon,
floramat,
florhydral,
gelsone,
geraniol,
hedione,
helional,
heliotropin,
heptanol,
hexahydro-4,7-methanoindan-1-carboxaldehyde,
hexahydro-8,8-dimethyl-2-naphthaldehyde,
hexalone,
hexyl salicylat,
hydroxycitronellal,
isoborneol,
isodamascone,
isoeugenol,
isojasmone,
isopulegol,
jasmacyclatat,
koavon,
laurinaldehyde,
ligustral,
lilial citral,
lilial,
limonen,
linalool,
linalyl acetate,
livescon,
lyral,
m-cymene-7-carboxaldehyde,
melonal,
melusat,
menthol,
menthone,
methyl acetophenone,
methyl cedrenyl ketone,
methyl cedrylon,
methyl cedrylone,
methyl cyclocitrone,
methyl dihydrojasmonate,
methyl heptenone,
methyl lavender ketone,
methyl-dihydrojasmonate,
methylnonyl acetaldehyde,
methyl-β-naphthyl ketone,
moschus indanone,
muscone,
myrtenol,
n-dodecanal,
neobutenone,
nerol,
n-hexanol,
nonanol,
n-undecanal,
octahydro-4,7-methano-11-1-indenecarboxaldehyde;
octanal,
octanol,
o-methoxyzimtaldehyde,
orivone,
p-ethyl-α,α-dimethylhydrozimtaldehyde,
phenol,
phenoxyacetaldehyde;
phenoxyisobutyrate,
phenyl acetaldehyde,
phenyl salicylat,
phenylethyl acetate,
phenylethyl alcohol,
phenylpropyl alcohol
p-hydroxy phenylbutanone,
pinen
plicatone,
p-menthan-7-ol,
p-methoxy acetophenone,
p-methyl phenoxy acetaldehyde,
p-tert.-bucinal,
p-tert.-butyl cyclohexanone,
p-tert.-butyl cyclohexylacetate,
p-tolyl acetaldehyde;
styrallyl propionate,
tetrahydrogeraniol,
tetrahydrolinalool,
tetrameran,
thymol,
tonalid/moschus plus,
trans-2-cis-6-nonadienol,
trans-2-nonen-1-ol,
trans-2-octenol,
trans-4-decenal,
triplal,
undecanal,
undecanol,
undecylenraldehyde,
vanillin,
veloutone,
verdone,
α-methyl-3,4-(methylenedioxy)-hydrocinnamaldehyde,
α-damascone,
α-methyl benzylalcohol,
α-methyl-4-(1-methylethyl)benzene-acetaldehyde,
α-methylphenylacetaldehyde,
α-amyl cinnamaldehyde,
α-n-hexyl cinnamaldehyde,
α-terpineol,
β-damascone,
β-ionon,
β-terpineol,
γ-methylionone,
δ-damascone.

### Cosmetic Preparations

Another object of the present invention refers to cosmetic preparations comprising the compositions as defined above. Said cosmetic preparations can represent skin care, personal care, sun care or hair care product or product formulation, comprising the composition as defined above in a working amount, for example about 0.01 to about 1 wt.-percent, preferably about 0.1 to about 0.5 wt.-percent calculated on the composition(s). The composition may represent for example a cosmetic cream, lotion, spray, emulsion, ointment, gel or mouse and the like.

The preparations according to the invention may contain antidandruff agents, irritation-preventing agents, irritation-inhibiting agents, antioxidants, adstringents, perspiration-inhibiting agents, antiseptic agents, ant-statics, binders, buffers, carrier materials, chelating agents, cell stimulants, cleansing agents, care agents, deodorizing agents, antiperspirants, softeners, emulsifiers, enzymes, essential oils, fibres, film-forming agents, fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, friction-reducing agents, lubricants, moisturizing creams, ointments, opacifying agents, plasticizing agents, covering agents, polish, gloss agents, polymers, powders, proteins, re-oiling agents, abrading agents, silicones, hair promotion agents, cooling agents, skin-cooling agents, warming agents, skin-warming agents, stabilizers, UV-absorbing agents, UV filters, detergents, thickeners, vitamins, oils, waxes, fats, phospholipids, saturated fatty acids, mono- or polyunsaturated fatty acids, α-hydroxy acids, polyhydroxyfatty acids, liquefiers, dyestuffs, colour-protecting agents, pigments, odoriferous substances, polyols, surfactants, electrolytes, organic solvents or silicone derivatives and the like as additional auxiliaries and additives.

### Surfactants

Preferred auxiliaries and additives are anionic and/or amphoteric or zwitterionic surfactants. Non-ionic and cationic surfactants can be also present in the composition. Suitable examples are mentioned along with the paragraph dealing with emulsifiers.

Typical examples for anionic and zwitterionic surfactants encompass: Almondamidopropylamine Oxide, Almondamidopropyl Betaine, Aminopropyl Laurylglutamine, Ammonium C12-15 Alkyl Sulfate, Ammonium C12-16 Alkyl Sulfate, Ammonium Capryleth Sulfate, Ammonium Cocomonoglyceride Sulfate, Ammonium Coco-Sulfate, Ammonium Cocoyl Isethionate, Ammonium Cocoyl Sarcosinate, Ammonium C12-15 Pareth Sulfate, Ammonium C9-10 Perfluoroalkylsulfonate, Ammonium Dinonyl Sulfosuccinate, Ammonium Dodecylbenzenesulfonate, Ammonium Isostearate, Ammonium Laureth-6 Carboxylate, Ammonium Laureth-8 Carboxylate, Ammonium Laureth Sulfate, Ammonium Laureth-5 Sulfate, Ammonium Laureth-7 Sulfate, Ammonium Laureth-9 Sulfate, Ammonium Laureth-12 Sulfate, Ammonium Lauroyl Sarcosinate, Ammonium Lauryl Sulfate, Ammonium Lauryl Sulfosuccinate, Ammonium Myreth Sulfate, Ammonium Myristyl Sulfate, Ammonium Nonoxynol-4 Sulfate, Ammonium Nonoxynol-30 Sulfate, Ammonium Oleate, Ammonium Palm Kernel Sulfate, Ammonium Stearate, Ammonium Tallate, AMPD-Isostearoyl Hydrolyzed Collagen, AMPD-Rosin Hydrolyzed Collagen, AMP-Isostearoyl Hydrolyzed Collagen, AMP-Isostearoyl Hydrolyzed Keratin, AMP-Isostearoyl Hydrolyzed Soy Protein, AMP-Isostearoyl Hydrolyzed Wheat Protein, Apricotamidopropyl Betaine, Arachidic Acid, Arginine Hexyldecyl Phosphate, Avocadamidopropyl Betaine, Avocado Oil Glycereth-8 Esters, Babassu Acid, Babassuamidopropylamine Oxide, Babassuamidopropyl Betaine, Beeswax Acid, Behenamidopropyl Betaine, Behenamine Oxide, Beheneth-25, Beheneth-30, Behenic Acid, Behenyl Betaine, Bis-Butyldimethicone Polyglyceryl-3, Butoxynol-5 Carboxylic Acid, Butoxynol-19 Carboxylic Acid, Butyldimoniumhydroxypropyl Butylglucosides Chloride, Butyldimoniumhydroxypropyl Laurylglucosides Chloride, Butyl Glucoside, Butylglucoside Caprate, Butylglucosides Hydroxypropyltrimonium Chloride, Butyloctanoic Acid, C18-36 Acid, C20-40 Acid, C30-50 Acid, C16-22 Acid Amide MEA, Calcium Dodecylbenzenesulfonate, Calcium Lauroyl Taurate, C9-16 Alkane/Cycloalkane, C10-14 Alkyl Benzenesulfonic Acid, C12-14 Alkyl Diaminoethylglycine HCL, C9-15 Alkyl Phosphate, Candida bombicola/Glucose/Methyl Rapeseedate Ferment, Canolamidopropyl Betaine, Capric Acid, Caproic Acid, Caproyl Ethyl Glucoside, Capryl/Capramidopropyl Betaine, Capryleth-4 Carboxylic Acid, Capryleth-6 Carboxylic Acid, Capryleth-9 Carboxylic Acid, Caprylic Acid, Capryloyl Collagen Amino Acids, Capryloyl Glycine, Capryloyl Hydrolyzed Collagen, Capryloyl Hydrolyzed Keratin, Capryloyl Keratin Amino Acids, Capryloyl Silk Amino Acids, Caprylyl/Capryl Glucoside, Caprylyl/Capryl Wheat Bran/Straw Glycosides, Caprylyl Glucoside, Caprylyl Glyceryl Ether, Caprylyl Pyrrolidone, Carnitine, Ceteareth-20, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Ceteareth-25 Carboxylic Acid, Ceteareth-2 Phosphate, Ceteareth-4 Phosphate, Ceteareth-5 Phosphate, Ceteareth-10 Phosphate, Ceteth-20, Ceteth-23, Ceteth-24, Ceteth-25, Ceteth-30, Ceteth-40, Ceteth-45, Ceteth-150, Ceteth-8 Phosphate, Ceteth-10 Phosphate, Ceteth-20 Phosphate, Cetoleth-22, Cetoleth-24, Cetoleth-25, Cetoleth-30, Cetyl Betaine, Chrysanthemum sinense Flower Extract, C12-14 Hydroxyalkyl Hydroxyethyl Beta-Alanine, C12-14 Hydroxyalkyl Hydroxyethyl Sarcosine, Cocamidoethyl Betaine, Cocamidopropylamine Oxide, Cocamidopropyl Betainamide MEA Chloride, Cocamidopropyl Betaine, Cocamidopropyl Hydroxysultaine, Cocamine Oxide, Cocaminobutyric Acid, Cocaminopropionic Acid, Coceth-7 Carboxylic Acid, Coceth-4 Glucoside, Cocoamphodipropionic Acid, Cocobetainamido Amphopropionate, Coco-Betaine, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Coco-Glucoside, Cocoglucosides Hydroxypropyltrimonium Chloride, Coco-Hydroxysultaine, Coco-Morpholine Oxide, Coconut Acid, Coconut Oil Glycereth-8 Esters, Coco/Oleamidopropyl Betaine, Coco-Sultaine, Coco/Sunfloweramidopropyl Betaine, Cocoylcholine Methosulfate, Cocoyl Glutamic Acid, Cocoyl Hydrolyzed Collagen, Cocoyl Hydrolyzed Keratin, Cocoyl Hydrolyzed Oat Protein, Cocoyl Hydrolyzed Rice Protein, Cocoyl Hydrolyzed Silk, Cocoyl Hydrolyzed Soy Protein, Cocoyl Hydrolyzed Wheat Protein, Cocoyl Sarcosine, Corn Acid, Cottonseed Acid, Cottonseed Oil Glycereth-8 Esters, C10-16 Pareth-1, C10-16 Pareth-2, C11-13 Pareth-6, C11-13 Pareth-9, C11-13 Pareth-10, C11-15 Pareth-30, C11-15 Pareth-40, C12-13 Pareth-1, C12-13 Pareth-23, C12-14 Pareth-5, C12-14 Pareth-9, C13-15 Pareth-21, C14-15 Pareth-8, C20-22 Pareth-30, C20-40 Pareth-40, C20-40 Pareth-95, C22-24 Pareth-33, C30-50 Pareth-40, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, C6-10 Pareth-4 Phosphate, C12-13 Pareth-2 Phosphate, C12-13 Pareth-10 Phosphate, C12-15 Pareth-6 Phosphate, C12-15 Pareth-8 Phosphate, C12-15 Pareth-10 Phosphate, C12-16 Pareth-6 Phosphate, C4-18 Perfluoroalkylethyl Thiohydroxypropyltrimonium Chloride, Cupuassuamidopropyl Betaine, DEA-C12-13 Alkyl Sulfate, DEA-C12-15 Alkyl Sulfate, DEA-Ceteareth-2 Phosphate, DEA-Cetyl Sulfate, DEA-Cocoamphodipropionate, DEA-C12-13 Pareth-3 Sulfate, DEA-Cyclocarboxypropyloleate, DEA-Dodecylbenzenesulfonate, DEA-Isostearate, DEA-Laureth Sulfate, DEA-Lauryl Sulfate, DEA-Linoleate, DEA-Methyl Myristate Sulfonate, DEA-Myreth Sulfate, DEA-Myristate, DEA-Myristyl Sulfate, DEA-Oleth-5 Phosphate, DEA-Oleth-20 Phosphate, DEA PG-Oleate, Deceth-7 Carboxylic Acid, Deceth-7 Glucoside, Deceth-9 Phosphate, Decylamine Oxide, Decyl Betaine, Decyl Glucoside, Decyltetradeceth-30, Decyltetradecylamine Oxide, Diammonium Lauramido-MEA Sulfosuccinate, Diammonium Lauryl Sulfosuccinate, Diammonium Oleamido PEG-2 Sulfosuccinate, Dibutoxymethane, Di-CI 2-15 Pareth-2 Phosphate, Di-CI 2-15 Pareth-4 Phosphate, Di-CI 2-15 Pareth-6 Phosphate, Di-C12-15 Pareth-8 Phosphate, Di-CI 2-15 Pareth-10 Phosphate, Didodecyl Butanetetracarboxylate, Diethylamine Laureth Sulfate, Diethylhexyl Sodium Sulfosuccinate, Dihydroxyethyl C8-10 Alkoxypropylamine Oxide, Dihydroxyethyl C9-11 Alkoxypropylamine Oxide, Dihydroxyethyl C12-15 Alkoxypropylamine Oxide, Dihydroxyethyl Cocamine Oxide, Dihydroxyethyl Lauramine Oxide, Dihydroxyethyl Stearamine Oxide, Dihydroxyethyl Tallowamine Oxide, Dimethicone PEG-7 Phosphate, Dimethicone PEG-10 Phosphate, Dimethicone PEG/PPG-7/4 Phosphate, Dimethicone PEG/PPG-12/4 Phosphate, Dimethicone/Polyglycerin-3 Crosspolymer, Dimethicone Propyl PG-Betaine, Dimyristyl Phosphate, Dioleoylamidoethyl Hydroxyethylmonium Methosulfate, DIPA-Hydrogenated Cocoate, DIPA-Lanolate, DIPA-Myristate, Dipotassium Capryloyl Glutamate, Dipotassium Lauryl Sulfosuccinate, Dipotassium Undecylenoyl Glutamate, Disodium Babassuamido MEA-Sulfosuccinate, Disodium Caproamphodiacetate, Disodium Caproamphodipropionate, Disodium Capryloamphodiacetate, Disodium Capryloamphodipropionate, Disodium Capryloyl Glutamate, Disodium Cetearyl Sulfosuccinate, Disodium Cetyl Phenyl Ether Disulfonate, Disodium Cetyl Sulfosuccinate, Disodium Cocamido MEA-Sulfosuccinate, Disodium Cocamido MIPA PEG-4 Sulfosuccinate, Disodium Cocamido MIPA-Sulfosuccinate, Disodium Cocamido PEG-3 Sulfosuccinate, Disodium Coceth-3 Sulfosuccinate, Disodium Cocoamphocarboxyethylhydroxypropylsulfonate, Disodium Cocoamphodiacetate, Disodium Cocoamphodipropionate, Disodium Coco-Glucoside Sulfosuccinate, Disodium Coco-Sulfosuccinate, Disodium Cocoyl Butyl Gluceth-10 Sulfosuccinate, Disodium Cocoyl Glutamate, Disodium C12-14 Pareth-1 Sulfosuccinate, Disodium C12-14 Pareth-2 Sulfosuccinate, Disodium C12-15 Pareth Sulfosuccinate, Disodium C12-14 Sec-Pareth-3 Sulfosuccinate, Disodium C12-14 Sec-Pareth-5 Sulfosuccinate, Disodium C12-14 Sec-Pareth-7 Sulfosuccinate, Disodium C12-14 Sec-Pareth-9 Sulfosuccinate, Disodium C12-14 Sec-Pareth-12 Sulfosuccinate, Disodium Deceth-5 Sulfosuccinate, Disodium Deceth-6 Sulfosuccinate, Disodium Decyl Phenyl Ether Disulfonate, Disodium Dihydroxyethyl Sulfosuccinylundecylenate, Disodium Ethylene Dicocamide PEG-15 Disulfate, Disodium Hydrogenated Cottonseed Glyceride Sulfosuccinate, Disodium Hydrogenated Tallow Glutamate, Disodium Hydroxydecyl Sorbitol Citrate, Disodium Isodecyl Sulfosuccinate, Disodium Isostearamido MEA-Sulfosuccinate, Disodium Isostearamido MIPA-Sulfosuccinate, Disodium Isostearoamphodiacetate, Disodium Isostearoamphodipropionate, Disodium isostearyl Sulfosuccinate, Disodium Laneth-5 Sulfosuccinate, Disodium Lauramido MEA-Sulfosuccinate, Disodium Lauramido MIPA Glycol Sulfosuccinate, Disodium Lauramido PEG-2 Sulfosuccinate, Disodium Lauramido PEG-5 Sulfosuccinate, Disodium Laureth-5 Carboxyamphodiacetate, Disodium Laureth-7 Citrate, Disodium Laureth Sulfosuccinate, Disodium Laureth-6 Sulfosuccinate, Disodium Laureth-9 Sulfosuccinate, Disodium Laureth-12 Sulfosuccinate, Disodium Lauriminobishydroxypropylsulfonate, Disodium Lauriminodiacetate, Disodium Lauriminodipropionate, Disodium Lauriminodipropionate Tocopheryl Phosphates, Disodium Lauroamphodiacetate, Disodium Lauroamphodipropionate, Disodium N-Lauroyl Aspartate, Disodium Lauroyl Glutamate, Disodium Lauryl Phenyl Ether Disulfonate, Disodium Lauryl Sulfosuccinate, Disodium Myristamido MEA-Sulfosuccinate, Disodium Nonoxynol-10 Sulfosuccinate, Disodium Oleamido MEA-Sulfosuccinate, Disodium Oleamido MIPA-Sulfosuccinate, Disodium Oleamido PEG-2 Sulfosuccinate, Disodium Oleoamphodipropionate, Disodium Oleth-3 Sulfosuccinate, Disodium Oleyl Phosphate, Disodium Oleyl Sulfosuccinate, Disodium Palmitamido PEG-2 Sulfosuccinate, Disodium Palmitoleamido PEG-2 Sulfosuccinate, Disodium PEG4 Cocamido MIPA-Sulfosuccinate, Disodium PEG-12 Dimethicone Sulfosuccinate, Disodium PEG-8 Palm Glycerides Sulfosuccinate, Disodium PPG-2-Isodeceth-7 Carboxyamphodiacetate, Disodium Ricinoleamido MEA-Sulfosuccinate, Disodium Sitostereth-14 Sulfosuccinate, Disodium Soyamphodiacetate, Disodium Stearamido MEA-Sulfosuccinate, Disodium Steariminodipropionate, Disodium Stearoamphodiacetate, Disodium Stearoyl Glutamate, Disodium Stearyl Sulfosuccinamate, Disodium Stearyl Sulfosuccinate, Disodium 2-Sulfolaurate, Disodium 2-Sulfopalmitate, Disodium Tallamido MEA-Sulfosuccinate, Disodium Tallowamido MEA-Sulfosuccinate, Disodium Tallowamphodiacetate, Disodium Tallowiminodipropionate, Disodium Tallow Sulfosuccinamate, Disodium Tridecylsulfosuccinate, Disodium Undecylenamido MEA-Sulfosuccinate, Disodium Undecylenamido PEG-2 Sulfosuccinate, Disodium Undecylenoyl Glutamate, Disodium Wheat Germamido MEA-Sulfosuccinate, Disodium Wheat Germamido PEG-2 Sulfosuccinate, Disodium Wheatgermamphodiacetate, Di-TEA-Cocamide Diacetate, Di-TEA-Oleamido PEG-2 Sulfosuccinate, Di-TEA-Palmitoyl Aspartate, Ditridecyl Sodium Sulfosuccinate, Dodecylbenzene Sulfonic Acid, Erucamidopropyl Hydroxysultaine, Ethylhex-eth-3 Carboxylic Acid, Ethyl PEG-15 Cocamine Sulfate, Glyceryl Capryl Ether, Hexyldecanoic Acid, Hydrogenated Coconut Acid, Hydrogenated Laneth-25, Hydrogenated Menhaden Acid, Hydrogenated Palm Acid, Hydrogenated Palm Kernel Amine Oxide, Hydrogenated Tallow Acid, Hydrogenated Tallowamine Oxide, Hydrogenated Tallow Betaine, Hydrogenated Talloweth-25, Hydrogenated Tallowoyl Glutamic Acid, Hydrolyzed Candida bombicola Extract, Hydroxyceteth-60, Hydroxyethyl Acetomonium PG-Dimethicone, Hydroxyethylbutylamine Laureth Sulfate, Hydroxyethyl Carboxymethyl Cocamidopropylamine, Hydroxyethyl Hydroxypropyl C12-15 Alkoxypropylamine Oxide, Hydroxylauryl/Hydroxymyristyl Betaine, Hydroxystearic Acid, Hydroxysuccinimidyl C10-40 Isoalkyl Acidate, Hydroxysuccinimidyl C21-22 Isoalkyl Acidate, Hydroxysultaines, IPDI/PEG-15 Soyamine Oxide Copolymer, IPDI/PEG-15 Soyethonium Ethosulfate Copolymer, IPDI/PEG-15 Soy Glycinate Copolymer, Isoceteth-30, Isolaureth-4 Phosphate, Isopolyglyceryl-3 Dimethicone, Isopolyglyceryl-3 Dimethiconol, Isopropanolamine Lanolate, Isopropylamine Dodecylbenzenesulfonate, Isostearamidopropylamine Oxide, Isostearamidopropyl Betaine, Isostearamidopropyl Morpholine Oxide, Isosteareth-8, Isosteareth-16, Isosteareth-22, Isosteareth-25, Isosteareth-50, Isostearic Acid, Isostearoyl Hydrolyzed Collagen, Jojoba Oil PEG-150 Esters, Jojoba Wax PEG-80 Esters, Jojoba Wax PEG-120 Esters, Laneth-20, Laneth-25, Laneth-40, Laneth-50, Laneth-60, Laneth-75, Lanolin Acid, Lauramidopropylamine Oxide, Lauramidopropyl Betaine, Lauramidopropyl Hydroxysultaine, Lauramine Oxide, Lauraminopropionic Acid, Laurdimoniumhydroxypropyl Decylglucosides Chloride, Laurdimoniumhydroxypropyl Laurylglucosides Chloride, Laureth-16, Laureth-20, Laureth-21, Laureth-23, Laureth-25, Laureth-30, Laureth-38, Laureth-40, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Laureth-6 Citrate, Laureth-7 Citrate, Laureth-1 Phosphate, Laureth-2 Phosphate, Laureth-3 Phosphate, Laureth-4 Phosphate, Laureth-7 Phosphate, Laureth-8 Phosphate, Laureth-7 Tartrate, Laurie Acid, Laurimino Bispropanediol, Lauriminodipropionic Acid, Lauroamphodipropionic Acid, Lauroyl Beta-Alanine, Lauroyl Collagen Amino Acids, Lauroyl Ethyltrimonium Methosulfate, Lauroyl Hydrolyzed Collagen, Lauroyl Hydrolyzed Elastin, Lauroyl Methyl Glucamide, Lauroyl Sarcosine, Lauroyl Silk Amino Acids, Lauryl Betaine, Lauryl Dimethicone/Polyglycerin-3 Crosspolymer, Lauryldimoniumhydroxypropyl Cocoglucosides Chloride, Lauryl Glucoside, Laurylglucosides Hydroxypropyltrimonium Chloride, Lauryl Glycol Hydroxypropyl Ether, Lauryl Hydroxysultaine, Lauryl Malamide, Lauryl Methylglucamide, Lauryl/Myristyl Glycol Hydroxypropyl Ether, Lauryl/Myristyl Wheat Bran/Straw Glycosides, Lauryl Polyglyceryl-3 Polydimethylsiloxyethyl Dimethicone, Lauryl Pyrrolidone, Lauryl Sultaine, Linoleic Acid, Linolenic Acid, Linseed Acid, Lysine Cocoate, Macadamia Seed Oil Glycereth-8 Esters, Magnesium Coceth Sulfate, Magnesium Coco-Sulfate, Magnesium Isododecylbenzenesulfonate, Magnesium Laureth-11 Carboxylate, Magnesium Laureth Sulfate, Magnesium Laureth-5 Sulfate, Magnesium Laureth-8 Sulfate, Magnesium Laureth-16 Sulfate, Magnesium Laureth-3 Sulfosuccinate, Magnesium Lauryl Hydroxypropyl Sulfonate, Magnesium Lauryl Sulfate, Magnesium Methyl Cocoyl Taurate, Magnesium Myreth Sulfate, Magnesium Oleth Sulfate, Magnesium/TEA-Coco-Sulfate, Manicouagan Clay, MEA-Cocoate, MEA-Laureth-6 Carboxylate, MEA-Laureth Sulfate, MEA-Lauryl Sulfate, MEA PPG-6 Laureth-7 Carboxylate, MEA-PPG-8-Steareth-7 Carboxylate, MEA-Undecylenate, Meroxapol 108, Meroxapol 174, Meroxapol 178, Meroxapol 254, Meroxapol 255, Meroxapol 258, Meroxapol 314, Methoxy PEG-450 Amidoglutaroyl Succinimide, Methoxy PEG-450 Amido Hydroxysuccinimidyl Succinamate, Methoxy PEG-450 Maleimide, Methyl Morpholine Oxide, Milkamidopropyl Amine Oxide, Milkamidopropyl Betaine, Minkamidopropylamine Oxide, Minkamidopropyl Betaine, MIPA C12-15 Pareth Sulfate, MIPA-Dodecylbenzenesulfonate, MIPA-Laureth Sulfate, MIPA-Lauryl Sulfate, Mixed Isopropanolamines Lanolate, Mixed Isopropanolamines Lauryl Sulfate, Mixed Isopropanolamines Myristate, Morpholine Oleate, Morpholine Stearate, Myreth-3 Carboxylic Acid, Myreth-5 Carboxylic Acid, Myristalkonium Chloride, Myristamidopropylamine Oxide, Myristamidopropyl Betaine, Myristamidopropyl Dimethylamine Phosphate, Myristamidopropyl Hydroxysultaine, Myristamidopropyl PG-Dimonium Chloride Phosphate, Myristamine Oxide, Myristaminopropionic Acid, Myristic Acid, Myristoyl Ethyltrimonium Methosulfate, Myristoyl Glutamic Acid, Myristoyl Hydrolyzed Collagen, Myristoyl Sarcosine, Myristyl Betaine, Myristyl/Cetyl Amine Oxide, Myristyldimoniumhydroxypropyl Cocoglucosides Chloride, Myristyl Glucoside, Myristyl Phosphate, Nonoxynol-20, Nonoxynol-23, Nonoxynol-25, Nonoxynol-30, Nonoxynol-35, Nonoxynol-40, Nonoxynol-44, Nonoxynol-50, Nonoxynol-100, Nonoxynol-120, Nonoxynol-5 Carboxylic Acid, Nonoxynol-8 Carboxylic Acid, Nonoxynol-10 Carboxylic Acid, Nonoxynol-3 Phosphate, Nonoxynol-4 Phosphate, Nonoxynol-6 Phosphate, Nonoxynol-9 Phosphate, Nonoxynol-10 Phosphate, Nonyl Nonoxynol-30, Nonyl Nonoxynol-49, Nonyl Nonoxynol-100, Nonyl Nonoxynol-150, Nonyl Nonoxynol-7 Phosphate, Nonyl Nonoxynol-8 Phosphate, Nonyl Nonoxynol-9 Phosphate, Nonyl Nonoxynol-10 Phosphate, Nonyl Nonoxynol-11 Phosphate, Nonyl Nonoxynol-15 Phosphate, Nonyl Nonoxynol-24 Phosphate, Oatamidopropyl Betaine, Octoxynol-16, Octoxynol-25, Octoxynol-30, Octoxynol-33, Octoxynol-40, Octoxynol-70, Octoxynol-20 Carboxylic Acid, Octyldodeceth-20, Octyldodeceth-25, Octyldodeceth-30, Oleamidopropylamine Oxide, Oleamidopropyl Betaine, Oleamidopropyl Hydroxysultaine, Oleamine Oxide, Oleic Acid, Oleoyl Hydrolyzed Collagen, Oleoyl Sarcosine, Oleth-20, Oleth-23, Oleth-24, Oleth-25, Oleth-30, Oleth-35, Oleth-40, Oleth-44, Oleth-50, Oleth-3 Carboxylic Acid, Oleth-6 Carboxylic Acid, Oleth-10 Carboxylic Acid, Oleyl Betaine, Olivamidopropylamine Oxide, Olivamidopropyl Betaine, Olive Acid, Olivoyl Hydrolyzed Wheat Protein, Ophiopogon Extract Stearate, Ozonized Oleth-10, Ozonized PEG-10 Oleate, Ozonized PEG-14 Oleate, Ozonized Polysorbate 80, Palm Acid, Palmamidopropyl Betaine, Palmeth-2 Phosphate, Palmitamidopropylamine Oxide, Palmitamidopropyl Betaine, Palmitamine Oxide, Palmitic Acid, Palmitoyl Collagen Amino Acids, Palmitoyl Glycine, Palmitoyl Hydrolyzed Collagen, Palmitoyl Hydrolyzed Milk Protein, Palmitoyl Hydrolyzed Wheat Protein, Palmitoyl Keratin Amino Acids, Palmitoyl Oligopeptide, Palmitoyl Silk Amino Acids, Palm Kernel Acid, Palm Kernelamidopropyl Betaine, Peach Kernel Oil Glycereth-8 Esters, Peanut Acid, PEG-10 Castor Oil, PEG-40 Castor Oil, PEG-44 Castor Oil, PEG-50 Castor Oil, PEG-54 Castor Oil, PEG-55 Castor Oil, PEG-60 Castor Oil, PEG-80 Castor Oil, PEG-100 Castor Oil, PEG-200 Castor Oil, PEG-11 Cocamide, PEG-6 Cocamide Phosphate, PEG-4 Cocamine, PEG-8 Cocamine, PEG-12 Cocamine, PEG-150 Dibehenate, PEG-90 Diisostearate, PEG-75 Dilaurate, PEG-150 Dilaurate, PEG-75 Dioleate, PEG-150 Dioleate, PEG-75 Distearate, PEG-120 Distearate, PEG-150 Distearate, PEG-175 Distearate, PEG-190 Distearate, PEG-250 Distearate, PEG-30 Glyceryl Cocoate, PEG-40 Glyceryl Cocoate, PEG-78 Glyceryl Cocoate, PEG-80 Glyceryl Cocoate, PEG-30 Glyceryl Isostearate, PEG-40 Glyceryl Isostearate, PEG-50 Glyceryl Isostearate, PEG-60 Glyceryl Isostearate, PEG-90 Glyceryl Isostearate, PEG-23 Glyceryl Laurate, PEG-30 Glyceryl Laurate, PEG-25 Glyceryl Oleate, PEG-30 Glyceryl Oleate, PEG-30 Glyceryl Soyate, PEG-25 Glyceryl Stearate, PEG-30 Glyceryl Stearate, PEG-40 Glyceryl Stearate, PEG-120 Glyceryl Stearate, PEG-200 Glyceryl Stearate, PEG-28 Glyceryl Tallowate, PEG-80 Glyceryl Tallowate, PEG-82 Glyceryl Tallowate, PEG-130 Glyceryl Tallowate, PEG-200 Glyceryl Tallowate, PEG-45 Hydrogenated Castor Oil, PEG-50 Hydrogenated Castor Oil, PEG-54 Hydrogenated Castor Oil, PEG-55 Hydrogenated Castor Oil, PEG-60 Hydrogenated Castor Oil, PEG-80 Hydrogenated Castor Oil, PEG-100 Hydrogenated Castor Oil, PEG-200 Hydrogenated Castor Oil, PEG-30 Hydrogenated Lanolin, PEG-70 Hydrogenated Lanolin, PEG-50 Hydrogenated Palmamide, PEG-2 Isostearate, PEG-3 Isostearate, PEG-4 Isostearate, PEG-6 Isostearate, PEG-8 Isostearate, PEG-10 Isostearate, PEG-12 Isostearate, PEG-20 Isostearate, PEG-30 Isostearate, PEG-40 Isostearate, PEG-26 Jojoba Acid, PEG-40 Jojoba Acid, PEG-15 Jojoba Alcohol, PEG-26 Jojoba Alcohol, PEG-40 Jojoba Alcohol, PEG-35 Lanolin, PEG-40 Lanolin, PEG-50 Lanolin, PEG-55 Lanolin, PEG60 Lanolin, PEG-70 Lanolin, PEG-75 Lanolin, PEG-85 Lanolin, PEG-100 Lanolin, PEG-150 Lanolin, PEG-75 Lanolin Oil, PEG-2 Lauramide, PEG-3 Lauramine Oxide, PEG-20 Laurate, PEG-32 Laurate, PEG-75 Laurate, PEG-150 Laurate, PEG-70 Mango Glycerides, PEG-20 Mannitan Laurate, PEG-8 Methyl Ether Dimethicone, PEG-120 Methyl Glucose Dioleate, PEG-80 Methyl Glucose Laurate, PEG120 Methyl Glucose Trioleate, PEG-4 Montanate, PEG-30 Oleamine, PEG-20 Oleate, PEG-23 Oleate, PEG-32 Oleate, PEG-36 Oleate, PEG-75 Oleate, PEG-150 Oleate, PEG-20 Palmitate, PEG-150 Polyglyceryl-2 Tristearate, PEG/PPG-28/21 Acetate Dimethicone, PEG/PPG-24/18 Butyl Ether Dimethicone, PEG/PPG-3/17 Copolymer, PEG/PPG-5/35 Copolymer, PEG/PPG-8/55 Copolymer, PEG/PPG-10/30 Copolymer, PEG/PPG-10/65 Copolymer, PEG/PPG-12/35 Copolymer, PEG/PPG-16/17 Copolymer, PEG/PPG-20/9 Copolymer, PEG/PPG-20/20 Copolymer, PEG/PPG-20/60 Copolymer, PEG/PPG-20/65 Copolymer, PEG/PPG-22/25 Copolymer, PEG/PPG-28/30 Copolymer, PEG/PPG-30-35 Copolymer, PEG/PPG-30/55 Copolymer, PEG/PPG-35/40 Copolymer, PEG/PPG-50/40 Copolymer, PEG/PPG-150/35 Copolymer, PEG/PPG-160/30 Copolymer, PEG/PPG-190/60 Copolymer, PEG/PPG-200/40 Copolymer, PEG/PPG-300/55 Copolymer, PEG/PPG-20/22 Methyl Ether Dimethicone, PEG26-PPG-30 Phosphate, PEG/PPG-4/2 Propylheptyl Ether, PEG/PPG-6/2 Propylheptyl Ether, PEG7/PPG-2 Propylheptyl Ether, PEG/PPG-8/2 Propylheptyl Ether, PEG/PPG-10/2 Propylheptyl Ether, PEG/PPG-14/2 Propylheptyl Ether, PEG/PPG-40/2 Propylheptyl Ether, PEG/PPG-10/2 Ricinoleate, PEG/PPG-32/3 Ricinoleate, PEG-55 Propylene Glycol Oleate, PEG-25 Propylene Glycol Stearate, PEG-75 Propylene Glycol Stearate, PEG-120 Propylene Glycol Stearate, PEG-5 Rapeseed Sterol, PEG10 Rapeseed Sterol, PEG-40 Ricinoleamide, PEG-75 Shea Butter Glycerides, PEG-75 Shorea Butter Glycerides, PEG-20 Sorbitan Cocoate, PEG-20 Sorbitan Isostearate, PEG-40 Sorbitan Lanolate, PEG75 Sorbitan Lanolate, PEG-10 Sorbitan Laurate, PEG-40 Sorbitan Laurate, PEG-44 Sorbitan Laurate, PEG-75 Sorbitan Laurate, PEG-80 Sorbitan Laurate, PEG-20 Sorbitan Oleate, PEG-80 Sorbitan Palmitate, PEG-40 Sorbitan Stearate, PEG-60 Sorbitan Stearate, PEG-160 Sorbitan Triisostearate, PEG-40 Soy Sterol, PEG-2 Stearamide Carboxylic Acid, PEG-9 Stearamide Carboxylic Acid, PEG-20 Stearate, PEG-23 Stearate, PEG-25 Stearate, PEG-30 Stearate, PEG-32 Stearate, PEG-35 Stearate, PEG-36 Stearate, PEG-40 Stearate, PEG-45 Stearate, PEG-50 Stearate, PEG-55 Stearate, PEG-75 Stearate, PEG-90 Stearate, PEG-100 Stearate, PEG-120 Stearate, PEG-150 Stearate, PEG-45 Stearate Phosphate, PEG20 Tallate, PEG-50 Tallow Amide, PEG-2 Tallowamide DEA, PEG-20 Tallowate, PEG-66 Trihydroxystearin, PEG-200 Trihydroxystearin, PEG-60 Tsubakiate Glycerides, Pelargonic Acid, Pentadoxynol-200, Pheneth-6 Phosphate, Poloxamer 105, Poloxamer 108, Poloxamer 182, Poloxamer 183, Poloxamer 184, Poloxamer 188, Poloxamer 217, Poloxamer 234, Poloxamer 235, Poloxamer 237, Poloxamer 238, Poloxamer 288, Poloxamer 334, Poloxamer 335, Poloxamer 338, Poloxamine 908, Poloxamine 1508, Polydimethylsiloxy PEG/PPG-24/19 Butyl Ether Silsesquioxane, Polydimethylsiloxy PPG-13 Butyl Ether Silsesquioxane, Polyglyceryl-6 Caprate, Polyglyceryl-10 Dilaurate, Polyglyceryl-20 Heptacaprylate, Polyglyceryl-20 Hexacaprylate, Polyglyceryl-2 Lauryl Ether, Polyglyceryl-10 Lauryl Ether, Polyglyceryl-20 Octaisononanoate, Polyglyceryl-6 Pentacaprylate, Polyglyceryl-10 Pentacaprylate, Polyglyceryl-3 Polydimethylsiloxyethyl Dimethicone, Polyglyceryl-6 Tetracaprylate, Polyglyceryl-10 Tetralaurate, Polyglyceryl-6 Tricaprylate, Polyglyceryl-10 Trilaurate, Polyquaternium-77, Polyquaternium-78, Polyquaternium-79, Polyquaternium-80, Polyquaternium-81, Polyquaternium-82, Pomaderris Kumerahou Flower/Leaf Extract, Poria Cocos Extract, Potassium Abietoyl Hydrolyzed Collagen, Potassium Babassuate, Potassium Behenate, Potassium C9-15 Alkyl Phosphate, Potassium C11-15 Alkyl Phosphate, Potassium C12-13 Alkyl Phosphate, Potassium C12-14 Alkyl Phosphate, Potassium Caprate, Potassium Capryloyl Glutamate, Potassium Capryloyl Hydrolyzed Rice Protein, Potassium Castorate, Potassium Cocoate, Potassium Cocoyl Glutamate, Potassium Cocoyl Glycinate, Potassium Cocoyl Hydrolyzed Casein, Potassium Cocoyl Hydrolyzed Collagen, Potassium Cocoyl Hydrolyzed Corn Protein, Potassium Cocoyl Hydrolyzed Keratin, Potassium Cocoyl Hydrolyzed Oat Protein, Potassium Cocoyl Hydrolyzed Potato Protein, Potassium Cocoyl Hydrolyzed Rice Bran Protein, Potassium Cocoyl Hydrolyzed Rice Protein, Potassium Cocoyl Hydrolyzed Silk, Potassium Cocoyl Hydrolyzed Soy Protein, Potassium Cocoyl Hydrolyzed Wheat Protein, Potassium Cocoyl Hydrolyzed Yeast Protein, Potassium Cocoyl PCA, Potassium Cocoyl Sarcosinate, Potassium Cocoyl Taurate, Potassium Cornate, Potassium Cyclocarboxypropyloleate, Potassium Dihydroxyethyl Cocamine Oxide Phosphate, Potassium Dimethicone PEG-7 Phosphate, Potassium Dodecylbenzenesulfonate, Potassium Hempseedate, Potassium Hydrogenated Cocoate, Potassium Hydrogenated Palmate, Potassium Hydrogenated Tallowate, Potassium Hydroxystearate, Potassium Isostearate, Potassium Lanolate, Potassium Laurate, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Laureth Phosphate, Potassium Lauroyl Collagen Amino Acids, Potassium Lauroyl Glutamate, Potassium Lauroyl Hydrolyzed Collagen, Potassium Lauroyl Hydrolyzed Pea Protein, Potassium Lauroyl Hydrolyzed Soy Protein, Potassium Lauroyl PCA, Potassium Lauroyl Pea Amino Acids, Potassium Lauroyl Sarcosinate, Potassium Lauroyl Silk Amino Acids, Potassium Lauroyl Wheat Amino Acids, Potassium Lauryl Phosphate, Potassium Lauryl Sulfate, Potassium Linoleate, Potassium Metaphosphate, Potassium Methyl Cocoyl Taurate, Potassium Myristate, Potassium Myristoyl Glutamate, Potassium Myristoyl Hydrolyzed Collagen, Potassium Octoxynol-12 Phosphate, Potassium Oleate, Potassium Oleoyl Hydrolyzed Collagen, Potassium Olivate, Potassium Olivoyl Hydrolyzed Oat Protein, Potassium Olivoyl Hydrolyzed Wheat Protein, Potassium Olivoyl/Lauroyl Wheat Amino Acids, Potassium Olivoyl PCA, Potassium Palmate, Potassium Palmitate, Potassium Palmitoyl Hydrolyzed Corn Protein, Potassium Palmitoyl Hydrolyzed Oat Protein, Potassium Palmitoyl Hydrolyzed Rice Protein, Potassium Palmitoyl Hydrolyzed Sweet Almond Protein, Potassium Palmitoyl Hydrolyzed Wheat Protein, Potassium Palm Kernelate, Potassium Peanutate, Potassium Rapeseedate, Potassium Ricinoleate, Potassium Safflowerate, Potassium Soyate, Potassium Stearate, Potassium Stearoyl Hydrolyzed Collagen, Potassium Tallate, Potassium Tallowate, Potassium Taurate, Potassium Taurine Laurate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, Potassium Trideceth-6 Phosphate, Potassium Trideceth-7 Phosphate, Potassium Tsubakiate, Potassium Undecylenate, Potassium Undecylenoyl Hydrolyzed Collagen, Potassium Undecylenoyl Hydrolyzed Rice Protein, PPG-30-Buteth-30, PPG-36-Buteth-36, PPG-38-Buteth-37, PPG-30-Capryleth-4 Phosphate, PPG-10 Cetyl Ether Phosphate, PPG-2 C9-11 Pareth-8, PPG-1-Deceth-5, PPG-3-Deceth-2 Carboxylic Acid, PPG-30 Ethylhexeth-4 Phosphate, PPG-20-Glycereth-30, PPG-2 Hydroxyethyl Coco/Isostearamide, PPG-2-Isodeceth-8, PPG-2-Isodeceth-10, PPG-2-Isodeceth-18, PPG-2-Isodeceth-25, PPG-4-Isodeceth-10, Propyltrimonium Hydrolyzed Collagen, Quaternium-24, Quaternium-52, Quaternium-87, Rapeseed Acid, Rice Bran Acid, Rice Oil Glycereth-8 Esters, Ricinoleamidopropyl Betaine, Ricinoleic Acid, Ricinoleth-40, Safflower Acid, Sapindus oahuensis Fruit Extract, Saponaria officinalis Root Powder, Saponins, Sekken-K, Sekken-Na/K, Sekken Soji, Sekken Soji-K, Sesame Oil Glycereth-8 Esters, Sesamidopropylamine Oxide, Sesamidopropyl Betaine, Shea Butteramidopropyl Betaine, Shea Butter Glycereth-8 Esters, Sodium Arachidate, Sodium Arganampohoacetate, Sodium Astrocaryum Murumuruate, Sodium Avocadoate, Sodium Babassuamphoacetate, Sodium Babassuate, Sodium Babassu Sulfate, Sodium Behenate, Sodium Bisglycol Ricinosulfosuccinate, Sodium Bis-Hydroxyethylglycinate CocoGlucosides Crosspolymer, Sodium Bis-Hydroxyethylglycinate Lauryl-Glucosides Crosspolymer, Sodium Borageamidopropyl PG-Dimonium Chloride Phosphate, Sodium Butoxynol-12 Sulfate, Sodium Butylglucosides Hydroxypropyl Phosphate, Sodium C13-17 Alkane Sulfonate, Sodium C14-18 Alkane Sulfonate, Sodium C12-15 Alkoxypropyl Iminodipropionate, Sodium C10-16 Alkyl Sulfate, Sodium C11-15 Alkyl Sulfate, Sodium C12-13 Alkyl Sulfate, Sodium C12-15 Alkyl Sulfate, Sodium C12-18 Alkyl Sulfate, Sodium C16-20 Alkyl Sulfate, Sodium C9-22 Alkyl Sec Sulfonate, Sodium C14-17 Alkyl Sec Sulfonate, Sodium Caprate, Sodium Caproamphoacetate, Sodium Caproamphohydroxypropylsulfonate, Sodium Caproamphopropionate, Sodium Caproyl Methyltaurate, Sodium Caprylate, Sodium Capryleth-2 Carboxylate, Sodium Capryleth-9 Carboxylate, Sodium Capryloamphoacetate, Sodium Capryloamphohydroxypropylsulfonate, Sodium Capryloamphopropionate, Sodium Capryloyl Glutamate, Sodium Capryloyl Hydrolyzed Wheat Protein, Sodium Caprylyl PG-Sulfonate, Sodium Caprylyl Sulfonate, Sodium Castorate, Sodium Ceteareth-13 Carboxylate, Sodium Cetearyl Sulfate, Sodium Ceteth-13 Carboxylate, Sodium Cetyl Sulfate, Sodium Cocamidopropyl PG-Dimonium Chloride Phosphate, Sodium Cocaminopropionate, Sodium Coceth Sulfate, Sodium Coceth-30 Sulfate, Sodium Cocoabutteramphoacetate, Sodium Cocoa Butterate, Sodium Cocoamphoacetate, Sodium Cocoamphohydroxypropylsulfonate, Sodium Cocoamphopropionate, Sodium Cocoate, Sodium Coco/Babassu/Andiroba Sulfate, Sodium Coco/Babassu Sulfate, Sodium Cocoglucosides Hydroxypropyl Phosphate, Sodium Cocoglucosides Hydroxypropylsulfonate, Sodium Coco-Glucoside Tartrate, Sodium Cocoglyceryl Ether Sulfonate, Sodium Coco/Hydrogenated Tallow Sulfate, Sodium Cocoiminodiacetate, Sodium Cocomonoglyceride Sulfate, Sodium Cocomonoglyceride Sulfonate, Sodium Coco PG-Dimonium Chloride Phosphate, Sodium Coco-Sulfate, Sodium Coco Sulfoacetate, Sodium Cocoyl Alaninate, Sodium Cocoyl Amino Acids, Sodium Cocoyl Collagen Amino Acids, Sodium Cocoyl Glutamate, Sodium Cocoyl Glutaminate, Sodium Cocoyl Glycinate, Sodium Cocoyl/Hydrogenated Tallow Glutamate, Sodium Cocoyl Hydrolyzed Collagen, Sodium Cocoyl Hydrolyzed Keratin, Sodium Cocoyl Hydrolyzed Rice Protein, Sodium Cocoyl Hydrolyzed Silk, Sodium Cocoyl Hydrolyzed Soy Protein, Sodium Cocoyl Hydrolyzed Sweet Almond Protein, Sodium Cocoyl Hydrolyzed Wheat Protein, Sodium Cocoyl Hydrolyzed Wheat Protein Glutamate, Sodium Cocoyl Isethionate, Sodium Cocoyl Methylaminopropionate, Sodium Cocoyl Oat Amino Acids, Sodium Cocoyl/Palmoyl/Sunfloweroyl Glutamate, Sodium Cocoyl Proline, Sodium Cocoyl Sarcosinate, Sodium Cocoyl Taurate, Sodium Cocoyl Threoninate, Sodium Cocoyl Wheat Amino Acids, Sodium C12-14 Olefin Sulfonate, Sodium C14-16 Olefin Sulfonate, Sodium C14-18 Olefin Sulfonate, Sodium C16-18 Olefin Sulfonate, Sodium Cornamphopropionate, Sodium Cottonseedamphoacetate, Sodium C13-15 Pareth-8 Butyl Phosphate, Sodium C9-11 Pareth-6 Carboxylate, Sodium C11-15 Pareth-7 Carboxylate, Sodium C12-13 Pareth-5 Carboxylate, Sodium C12-13 Pareth-8 Carboxylate, Sodium C12-13 Pareth-12 Carboxylate, Sodium C12-15 Pareth-6 Carboxylate, Sodium C12-15 Pareth-7 Carboxylate, Sodium C12-15 Pareth-8 Carboxylate, Sodium C14-15 Pareth-8 Carboxylate, Sodium C12-14 Sec-Pareth-8 Carboxylate, Sodium C14-15 Pareth-PG Sulfonate, Sodium C12-13 Pareth-2 Phosphate, Sodium C13-15 Pareth-8 Phosphate, Sodium C9-15 Pareth-3 Sulfate, Sodium C10-15 Pareth Sulfate, Sodium C10-16 Pareth-2 Sulfate, Sodium C12-13 Pareth Sulfate, Sodium C12-15 Pareth Sulfate, Sodium C12-15 Pareth-3 Sulfate, Sodium C13-15 Pareth-3 Sulfate, Sodium C12-14 Sec-Pareth-3 Sulfate, Sodium C12-15 Pareth-3 Sulfonate, Sodium C12-15 Pareth-7 Sulfonate, Sodium C12-15 Pareth-15 Sulfonate, Sodium Deceth-2 Carboxylate, Sodium Deceth Sulfate, Sodium Decylbenzenesulfonate, Sodium Decylglucosides Hydroxypropyl Phosphate, Sodium Decylglucosides Hydroxypropylsulfonate, Sodium Dilaureth-7 Citrate, Sodium Dilaureth-10 Phosphate, Sodium Dilinoleamidopropyl PG-Dimonium Chloride Phosphate, Sodium Dilinoleate, Sodium Dioleth-8 Phosphate, Sodium Dodecylbenzenesulfonate, Sodium Ethyl 2-Sulfolaurate, Sodium Glyceryl Oleate Phosphate, Sodium Grapeseedamidopropyl PG-Dimonium Chloride Phosphate, Sodium Grapeseedamphoacetate, Sodium Grapeseedate, Sodium Hempseedamphoacetate, Sodium Hexeth-4 Carboxylate, Sodium Hydrogenated Cocoate, Sodium Hydrogenated Cocoyl Methyl Isethionate, Sodium Hydrogenated Palmate, Sodium Hydrogenated Tallowate, Sodium Hydrogenated Tallowoyl Glutamate, Sodium Hydroxylauryldimonium Ethyl Phosphate, Sodium Hydroxypropyl Palm Kernelate Sulfonate, Sodium Hydroxypropylphosphate Decylglucoside Crosspolymer, Sodium Hydroxypropylphosphate Laurylglucoside Crosspolymer, Sodium Hydroxypropylsulfonate Cocoglucoside Crosspolymer, Sodium Hydroxypropylsulfonate Decylglucoside Crosspolymer, Sodium Hydroxypropylsulfonate Laurylglucoside Crosspolymer, Sodium Hydroxystearate, Sodium Isostearate, Sodium Isosteareth-6 Carboxylate, Sodium Isosteareth-11 Carboxylate, Sodium Isostearoamphoacetate, Sodium Isostearoamphopropionate, Sodium N-Isostearoyl Methyltaurate, Sodium Laneth Sulfate, Sodium Lanolate, Sodium Lardate, Sodium Lauramido Diacetate, Sodium Lauraminopropionate, Sodium Laurate, Sodium Laureth-3 Carboxylate, Sodium Laureth-4 Carboxylate, Sodium Laureth-5 Carboxylate, Sodium Laureth-6 Carboxylate, Sodium Laureth-8 Carboxylate, Sodium Laureth-11 Carboxylate, Sodium Laureth-12 Carboxylate, Sodium Laureth-13 Carboxylate, Sodium Laureth-14 Carboxylate, Sodium Laureth-16 Carboxylate, Sodium Laureth-17 Carboxylate, Sodium Laureth Sulfate, Sodium Laureth-5 Sulfate, Sodium Laureth-7 Sulfate, Sodium Laureth-8 Sulfate, Sodium Laureth-12 Sulfate, Sodium Laureth-40 Sulfate, Sodium Laureth-7 Tartrate, Sodium Lauriminodipropionate, Sodium Lauroamphoacetate, Sodium Lauroamphohydroxypropylsulfonate, Sodium Lauroampho PG-Acetate Phosphate, Sodium Lauroamphopropionate, Sodium Lauroyl Aspartate, Sodium Lauroyl Collagen Amino Acids, Sodium Lauroyl Glycine Propionate, Sodium Lauroyl Hydrolyzed Collagen, Sodium Lauroyl Hydrolyzed Silk, Sodium Lauroyl Hydroxypropyl Sulfonate, Sodium Lauroyl Isethionate, Sodium Lauroyl Methylaminopropionate, Sodium Lauroyl Methyl Isethionate, Sodium Lauroyl Millet Amino Acids, Sodium Lauroyl/Myristoyl Aspartate, Sodium Lauroyl Oat Amino Acids, Sodium Lauroyl Sarcosinate, Sodium Lauroyl Silk Amino Acids, Sodium Lauroyl Taurate, Sodium Lauroyl Wheat Amino Acids, Sodium Lauryl Diethylenediaminoglycinate, Sodium Lauryl Glucose Carboxylate, Sodium Laurylglucosides Hydroxypropyl Phosphate, Sodium Laurylglucosides Hydroxypropylsulfonate, Sodium Lauryl Glycol Carboxylate, Sodium Lauryl Hydroxyacetamide Sulfate, Sodium Lauryl Phosphate, Sodium Lauryl Sulfate, Sodium Lauryl Sulfoacetate, Sodium Linoleate, Sodium Macadamiaseedate, Sodium Mangoamphoacetate, Sodium Mangoseedate, Sodium/MEA Laureth-2 Sulfosuccinate, Sodium Methoxy PPG-2 Acetate, Sodium Methyl Cocoyl Taurate, Sodium Methyl Lauroyl Taurate, Sodium Methyl Myristoyl Taurate, Sodium Methyl Oleoyl Taurate, Sodium Methyl Palmitoyl Taurate, Sodium Methyl Stearoyl Taurate, Sodium Methyl 2-Sulfolaurate, Sodium Methyl 2-Sulfopalmitate, Sodium Methyltaurate Isopalmitamide, Sodium Methyltaurine Cocoyl Methyltaurate, Sodium Myreth Sulfate, Sodium Myristate, Sodium Myristoamphoacetate, Sodium Myristoyl Glutamate, Sodium Myristoyl Hydrolyzed Collagen, Sodium Myristoyl Isethionate, Sodium Myristoyl Sarcosinate, Sodium Myristyl Sulfate, Sodium Nonoxynol-6 Phosphate, Sodium Nonoxynol-9 Phosphate, Sodium Nonoxynol-1 Sulfate, Sodium Nonoxynol-3 Sulfate, Sodium Nonoxynol-4 Sulfate, Sodium Nonoxynol-6 Sulfate, Sodium Nonoxynol-8 Sulfate, Sodium Nonoxynol-10 Sulfate, Sodium Nonoxynol-25 Sulfate, Sodium Octoxynol-2 Ethane Sulfonate, Sodium Octoxynol-2 Sulfate, Sodium Octoxynol-6 Sulfate, Sodium Octoxynol-9 Sulfate, Sodium Oleate, Sodium Oleoamphoacetate, Sodium Oleoamphohydroxypropylsulfonate, Sodium Oleoamphopropionate, Sodium Oleoyl Hydrolyzed Collagen, Sodium Oleoyl Isethionate, Sodium Oleth Sulfate, Sodium Oleyl Methyl Isethionate, Sodium Oleyl Sulfate, Sodium Olivamphoacetate, Sodium Olivate, Sodium Olivoyl Glutamate, Sodium Palmamphoacetate, Sodium Palmate, Sodium Palm Glyceride Sulfonate, Sodium Palmitate, Sodium Palmitoyl Hydrolyzed Collagen, Sodium Palmitoyl Hydrolyzed Wheat Protein, Sodium Palmitoyl Sarcosinate, Sodium Palm Kernelate, Sodium Palm Kerneloyl Isethionate, Sodium Palmoyl Glutamate, Sodium Passiflora edulis Seedate, Sodium Peanutamphoacetate, Sodium Peanutate, Sodium PEG-6 Cocamide Carboxylate, Sodium PEG-8 Cocamide Carboxylate, Sodium PEG-4 Cocamide Sulfate, Sodium PEG-3 Lauramide Carboxylate, Sodium PEG-4 Lauramide Carboxylate, Sodium PEG-8 Palm Glycerides Carboxylate, Sodium Pentaerythrityl Hydroxypropyl Iminodiacetate Dendrimer, Sodium Propoxy PPG-2 Acetate, Sodium Rapeseedate, Sodium Ricebranamphoacetate, Sodium Ricinoleate, Sodium Ricinoleoamphoacetate, Sodium Rose Hipsamphoacetate, Sodium Rosinate, Sodium Safflowerate, Sodium Saffloweroyl Hydrolyzed Soy Protein, Sodium Sesameseedate, Sodium Sesamphoacetate, Sodium Sheabutteramphoacetate, Sodium Soyate, Sodium Soy Hydrolyzed Collagen, Sodium Stearate, Sodium Stearoamphoacetate, Sodium Stearoamphohydroxypropylsulfonate, Sodium Stearoamphopropionate, Sodium Stearoyl Casein, Sodium Stearoyl Glutamate, Sodium Stearoyl Hyaluronate, Sodium Stearoyl Hydrolyzed Collagen, Sodium Stearoyl Hydrolyzed Corn Protein, Sodium Stearoyl Hydrolyzed Silk, Sodium Stearoyl Hydrolyzed Soy Protein, Sodium Stearoyl Hydrolyzed Wheat Protein, Sodium Stearoyl Lactalbumin, Sodium Stearoyl Methyl Isethionate, Sodium Stearoyl Oat Protein, Sodium Stearoyl Pea Protein, Sodium Stearoyl Soy Protein, Sodium Stearyl Dimethyl Glycine, Sodium Stearyl Sulfate, Sodium Sunflowerseedamphoacetate, Sodium Surfactin, Sodium Sweetalmondamphoacetate, Sodium Sweet Almondate, Sodium Tallamphopropionate, Sodium Tallate, Sodium Tallowamphoacetate, Sodium Tallowate, Sodium Tallow Sulfate, Sodium Tamanuseedate, Sodium Taurate, Sodium Taurine Cocoyl Methyltaurate, Sodium Taurine Laurate, Sodium/TEA-Lauroyl Collagen Amino Acids, Sodium/TEA-Lauroyl Hydrolyzed Collagen, Sodium/TEA-Lauroyl Hydrolyzed Keratin, Sodium/TEA-Lauroyl Keratin Amino Acids, Sodium/TEA-Undecylenoyl Collagen Amino Acids, Sodium/TEA-Undecylenoyl Hydrolyzed Collagen, Sodium/TEA-Undecylenoyl Hydrolyzed Corn Protein, Sodium/TEA-Undecylenoyl Hydrolyzed Soy Protein, Sodium/TEA-Undecylenoyl Hydrolyzed Wheat Protein, Sodium Theobroma grandiflorum Seedate, Sodium Trideceth-3 Carboxylate, Sodium Trideceth-4 Carboxylate, Sodium Trideceth-6 Carboxylate, Sodium Trideceth-7 Carboxylate, Sodium Trideceth-8 Carboxylate, Sodium Trideceth-12 Carboxylate, Sodium Trideceth-15 Carboxylate, Sodium Trideceth-19 Carboxylate, Sodium Trideceth Sulfate, Sodium Tridecylbenzenesulfonate, Sodium Tridecyl Sulfate, Sodium Trimethylolpropane Hydroxypropyl Iminodiacetate Dendrimer, Sodium Undeceth-5 Carboxylate, Sodium Undecylenate, Sodium Undecylenoamphoacetate, Sodium Undecylenoamphopropionate, Sodium Undecylenoyl Glutamate, Sodium Wheat Germamphoacetate, Sorbeth-160 Tristearate, Soy Acid, Soyamidopropylamine Oxide, Soyamidopropyl Betaine, Soybean Oil Glycereth-8 Esters, Stearamidopropylamine Oxide, Stearamidopropyl Betaine, Stearamine Oxide, Steareth-15, Steareth-16, Steareth-20, Steareth-21, Steareth-25, Steareth-27, Steareth-30, Steareth-40, Steareth-50, Steareth-80, Steareth-100, Steareth-2 Phosphate, Steareth-3 Phosphate, Stearic Acid, Stearoxypropyltrimonium Chloride, Stearoyl Glutamic Acid, Stearoyl Sarcosine, Stearyl Betaine, Stearyldimoniumhydroxypropyl Butylglucosides Chloride, Stearyldimoniumhydroxypropyl Decylglucosides Chloride, Stearyldimoniumhydroxypropyl Laurylglucosides Chloride, Sulfated Castor Oil, Sulfated Coconut Oil, Sulfated Glyceryl Oleate, Sulfated Olive Oil, Sulfated Peanut Oil, Sunfloweramide MEA, Sunflower Seed Acid, Sunflowerseedamidopropyl Hydroxyethyldimonium Chloride, Sunflower Seed Oil Glycereth-8 Esters, Tall Oil Acid, Tallow Acid, Tallowamidopropylamine Oxide, Tallowamidopropyl Betaine, Tallowamidopropyl Hydroxysultaine, Tallowamine Oxide, Tallow Betaine, Tallow Dihydroxyethyl Betaine, Tallowoyl Ethyl Glucoside, TEA-Abietoyl Hydrolyzed Collagen, TEA-C12-14 Alkyl Phosphate, TEA-C10-15 Alkyl Sulfate, TEA-C11-15 Alkyl Sulfate, TEA-C12-13 Alkyl Sulfate, TEA-C12-14 Alkyl Sulfate, TEA-C12-15 Alkyl Sulfate, TEA C14-17 Alkyl See Sulfonate, TEA-Canolate, TEA-Cocamide Diacetate, TEA-Cocoate, TEA-Coco-Sulfate, TEA-Cocoyl Alaninate, TEA-Cocoyl Glutamate, TEA-Cocoyl Glutaminate, TEA-Cocoyl Glycinate, TEA-Cocoyl Hydrolyzed Collagen, TEA-Cocoyl Hydrolyzed Soy Protein, TEA-Cocoyl Sarcosinate, TEA-Dimethicone PEG-7 Phosphate, TEA-Dodecylbenzenesulfonate, TEA-Hydrogenated Cocoate, TEA-Hydrogenated Tallowoyl Glutamate, TEA-Isostearate, TEA-Isostearoyl Hydrolyzed Collagen, TEA-Lauraminopropionate, TEA-Laurate, TEA-Laurate/Myristate, TEA-Laureth Sulfate, TEA-Lauroyl Collagen Amino Acids, TEA-Lauroyl Glutamate, TEA-Lauroyl Hydrolyzed Collagen, TEA-Lauroyl Keratin Amino Acids, TEA-Lauroyl Methylaminopropionate, TEA-Lauroyl/Myristoyl Aspartate, TEA-Lauroyl Sarcosinate, TEA-Lauryl Phosphate, TEA-Lauryl Sulfate, TEA-Myristaminopropionate, TEA-Myristate, TEA-Myristoyl Hydrolyzed Collagen, TEA-Oleate, TEA-Oleoyl Hydrolyzed Collagen, TEA-Oleoyl Sarcosinate, TEA-Oleyl Sulfate, TEA-Palmitate, TEA-Palm Kernel Sarcosinate, TEA-PEG-3 Cocamide Sulfate, TEA-Rosinate, TEA-Stearate, TEA-Tallate, TEA-Tridecylbenzenesulfonate, TEA-Undecylenate, TEA-Undecylenoyl Hydrolyzed Collagen, Tetramethyl Decynediol, Tetrasodium Dicarboxyethyl Stearyl Sulfosuccinamate, TIPA-Laureth Sulfate, TIPA-Lauryl Sulfate, TIPA-Myristate, TIPA-Stearate, Tocopheryl Phosphate, Trehalose Undecylenoate, TM-C12-15 Pareth-2 Phosphate, TM-C12-15 Pareth-6 Phosphate, TM-C12-15 Pareth-8 Phosphate, TM-C12-15 Pareth-10 Phosphate, Trideceth-20, Trideceth-50, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid, Trideceth-10 Phosphate, Tridecylbenzenesulfonic Acid, Trilaureth-9 Citrate, Trimethylolpropane Hydroxypropyl Bis-Hydroxyethylamine Dendrimer, Trisodium Lauroampho PG-Acetate Chloride Phosphate, Undecanoic Acid, Undeceth-5 Carboxylic Acid, Undecylenamidopropylamine Oxide, Undecylenamidopropyl Betaine, Undecylenic Acid, Undecylenoyl Collagen Amino Acids, Undecylenoyl Glycine, Undecylenoyl Hydrolyzed Collagen, Undecylenoyl Wheat Amino Acids, Undecyl Glucoside, Wheat Germ Acid, Wheat Germamidopropylamine Oxide, Wheat Germamidopropyl Betaine, Yucca schidigera Leaf/Root/Stem Extract, Yucca schidigera Stem Extract, Zinc Coceth Sulfatea and Zinc Coco-Sulfate.

Preferred are one or more compounds selected from the group consisting of Sodium Laureth Sulfate, Cocamidopropyl Betaine, Sodium Cocoamphoacetate, CocoGlucoside and Ammonium Lauryl Sulfosuccinate.

The percentage content of surfactants in the preparations may be from 0.1 to 10% by weight and is preferably from 0.5 to 5% by weight, based on the preparation.

### Oil Bodies

Suitable oil bodies, which form constituents of the O/W emulsions, are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxy carboxylic acids with linear or branched C₆-C₂₂-fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₅-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkyl-cyclohexanes.

### Emulsifiers

Other non-ionic or cationic surfactants may also be added to the preparations as emulsifiers, including for example:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows: Partial glycerides. Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters. Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters. Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan© GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable poly-olesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Tetraalkyl ammonium salts. Cationically active surfactants comprise the hydrophobic high molecular group required for the surface activity in the cation by dissociation in aqueous solution. A group of important representatives of the cationic surfactants are the tetraalkyl ammonium salts of the general formula: (R¹R²R³R⁴N⁺)X⁻. Here R1 stands for C₁-C₈ alk(en)yl, R², R³ and R⁴, independently of each other, for alk(en)yl radicals having 1 to 22 carbon atoms. X is a counter ion, preferably selected from the group of the halides, alkyl sulfates and alkyl carbonates. Cationic surfactants, in which the nitrogen group is substituted with two long acyl groups and two short alk(en)yl groups, are particularly preferred.

Esterquats. A further class of cationic surfactants particularly useful as co-surfactants for the present invention is represented by the so-called esterquats. Esterquats are generally understood to be quaternised fatty acid triethanolamine ester salts. These are known compounds which can be obtained by the relevant methods of preparative organic chemistry. Reference is made in this connection to International patent application WO 91/01295 A1, according to which triethanolamine is partly esterified with fatty acids in the presence of hypophosphorous acid, air is passed through the reaction mixture and the whole is then quaternised with dimethyl sulphate or ethylene oxide. In addition, German patent DE 4308794 C1 describes a process for the production of solid esterquats in which the quaternisation of triethanolamine esters is carried out in the presence of suitable dispersants, preferably fatty alcohols.

Typical examples of esterquats suitable for use in accordance with the invention are products of which the acyl component derives from monocarboxylic acids corresponding to formula RCOOH in which RCO is an acyl group containing 6 to 10 carbon atoms, and the amine component is triethanolamine (TEA). Examples of such monocarboxylic acids are caproic acid, caprylic acid, capric acid and technical mixtures thereof such as, for example, so-called head-fractionated fatty acid. Esterquats of which the acyl component derives from monocarboxylic acids containing 8 to 10 carbon atoms, are preferably used. Other esterquats are those of which the acyl component derives from dicarboxylic acids like malonic acid, succinic acid, maleic acid, fumaric acid, glutaric acid, sorbic acid, pimelic acid, azelaic acid, sebacic acid and/or dodecanedioic acid, but preferably adipic acid. Overall, esterquats of which the acyl component derives from mixtures of monocarboxylic acids containing 6 to 22 carbon atoms, and adipic acid are preferably used. The molar ratio of mono and dicarboxylic acids in the final esterquat may be in the range from 1:99 to 99:1 and is preferably in the range from 50:50 to 90:10 and more particularly in the range from 70:30 to 80:20. Besides the quaternised fatty acid triethanolamine ester salts, other suitable esterquats are quaternized ester salts of mono-/dicarboxylic acid mixtures with diethanolalkyamines or 1,2-dihydroxypropyl dialkylamines. The esterquats may be obtained both from fatty acids and from the corresponding triglycerides in admixture with the corresponding dicarboxylic acids. One such process, which is intended to be representative of the relevant prior art, is proposed in European patent EP 0750606 B1. To produce the quaternised esters, the mixtures of mono- and dicarboxylic acids and the triethanolamine—based on the available carboxyl functions—may be used in a molar ratio of 1.1:1 to 3:1. With the performance properties of the esterquats in mind, a ratio of 1.2:1 to 2.2:1 and preferably 1.5:1 to 1.9:1 has proved to be particularly advantageous. The preferred esterquats are technical mixtures of mono-, di- and triesters with an average degree of esterification of 1.5 to 1.9.

### Superfatting Agents and Consistency Factors

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

### Thickening Agents and Rheology Additives

Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

### Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine©, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar® CBS, Jaguar® C-17, Jaguar® C-16 of Celanese, quaternized ammonium salt polymers such as, for example, Mirapol® A-15, Mirapol® AD-1, Mirapol® AZ-1 of Miranol and the various polyquaternium types (for example 6, 7, 32 or 37) which can be found in the market under the tradenames Rheocare® CC or Ultragel® 300.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

### Pearlizing Waxes

Suitable pearlising waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

### Silicones

Suitable silicones can be chosen from the group consisting of: Acefylline Methylsilanol Mannuronate, Acetylmethionyl Methylsilanol Elastinate Acrylates/Behenyl, Acrylate/Dimethicone Methacrylate Copolymer, Acrylates/Behenyl Methacrylate/Dimethicone Methacrylate Copolymer, Acrylates/Bis-Hydroxypropyl Dimethicone Crosspolymer, Acrylates/Dimethicone Copolymer, Acrylates/Dimethicone Methacrylate/Ethylhexyl Acrylate Copolymer, Acrylates/Dimethiconol Acrylate Copolymer, Acrylates/Ethylhexyl Acrylate/Dimethicone Methacrylate Copolymer, Acrylates/Octylacrylamide/Diphenyl Amodimethicone Copolymer, Acrylates/Polytrimethylsiloxymethacrylate Copolymer, Acrylates/Propyl Trimethicone Methacrylate Copolymer, Acrylates/Stearyl Acrylate/Dimethicone Methacrylate Copolymer, Acrylates/Tridecyl Acrylate/Triethoxysilylpropyl Methacrylate/Dimethicone Methacrylate Copolymer, Acrylates/Trifluoropropylmethacrylate/Polytrimethyl Siloxymethacrylate Copolymer, Amino Bispropyl Dimethicone, Aminoethylaminopropyl Dimethicone, Aminopropyl Dimethicone, Aminopropyl Phenyl Trimethicone, Aminopropyl Triethoxysilane, Ammonium Dimethicone PEG-7 Sulfate, Amodimethicone, Amodimethicone Hydroxystearate, Amodimethicone/Silsesquioxane Copolymer, Ascorbyl Carboxydecyl Trisiloxane, Ascorbyl Methylsilanol Pectinate, Behenoxy Dimethicone, Behentrimonium Dimethicone PEG-8 Phthalate, Behenyl Dimethicone, Bisamino PEG/PPG-41/3 Aminoethyl PG-Propyl Dimethicone, Bis-Aminopropyl/Ethoxy Aminopropyl Dimethicone, Bis(Butylbenzoate) Diaminotriazine Aminopropyltrisiloxane, Bis-Butyldimethicone Polyglyceryl-3, Bis-Butyloxyamodimethicone/PEG-60 Copolymer, Bis(C13-15 Alkoxy) Hydroxybutamidoamodimethicone, Bis(C13-15 Alkoxy) PG-Amodimethicone, Bis-(C1-8 Alkyl Lauroyl Lysine Decylcarboxamide) Dimethicone, Bis-Cetyl Cetyl Dimethicone, Bis-Cetyl/PEG-8 Cetyl PEG-8 Dimethicone, Bis-Diphenylethyl Disiloxane, Bis-Ethyl Ethyl Methicone, Bis-Gluconamidoethylaminopropyl Dimethicone, Bis-Hydrogen Dimethicone, Bis-Hydroxyethoxypropyl Dimethicone Bis-Hydroxylauryl, Dimethicone/IPDI Copolymer, Bis-Hydroxy/Methoxy Amodimethicone, Bis-Hydroxypropyl Dimethicone Behenate, Bis-Hydroxypropyl Dimethicone/SMDI Copolymer, Bis-Isobutyl PEG-14/Amodimethicone Copolymer, Bis-Isobutyl PEG-15/Amodimethicone Copolymer, Bis-Isobutyl PEG/PPG-20/35/Amodimethicone Copolymer, Bis-Isobutyl PEG/PPG-10/7/Dimethicone Copolymer, Bis-Isobutyl PEG-24/PPG-7/Dimethicone Copolymer, Bis-PEG-1 Dimethicone, Bis-PEG-4 Dimethicone, Bis-PEG-8 Dimethicone, Bis-PEG-12 Dimethicone, Bis-PEG-20 Dimethicone, Bis-PEG-12 Dimethicone Beeswax, Bis-PEG-12 Dimethicone Candelillate, Bis-PEG-15 Dimethicone/IPDI Copolymer, Bis-PEG15 Methyl Ether Dimethicone, Bis-PEG-18 Methyl Ether Dimethyl Silane, Bis-PEG/PPG-14/14 Dimethicone, Bis-PEG/PPG-15/5 Dimethicone, Bis-PEG/PPG-18/6 Dimethicone, Bis-PEG/PPG-20/20 Dimethicone, Bis-PEG/PPG-16/16 PEG/PPG-16/16 Dimethicone, Bis-PEG/PPG-20/5 PEG/PPG-20/5 Dimethicone, Bisphenylhexamethicone, Bis-Phenylpropyl Dimethicone, Bispolyethylene Dimethicone, Bis-(Polyglyceryl-3 Oxyphenylpropyl) Dimethicone, Bis-(Polyglyceryl-7 Oxyphenylpropyl) Dimethicone, Bis-PPG-15 Dimethicone/IPDI Copolymer, Bis(PPG-7 Undeceneth-21) Dimethicone, Bis-Stearyl Dimethicone, Bis-Trimethoxysilylethyl Tetramethyldisiloxyethyl Dimethicone, Bis-Vinyldimethicone, Bis-Vinyl Dimethicone/Dimethicone Copolymer, Borage Seed Oil PEG-7 Dimethicone Esters, Butyl Acrylate/C6-14 Perfluoroalkylethyl Acrylate/Mercaptopropyl Dimethicone Copolymer, Butyl Acrylate/Hydroxypropyl Dimethicone Acrylate Copolymer, Butyl Dimethicone Acrylate/Cyclohexylmethacrylate/Ethylhexyl Acrylate Copolymer, Butyldimethicone Methacrylate/Methyl Methacrylate Crosspolymer, t-Butyl Dimethyl SiIyI Grape Seed Extract, Butyl Polydimethylsiloxyl Ethylene/Propylene/Vinylnorbornene Copolymer, C6-8 Alkyl C3-6 Alkyl Glucoside Dimethicone, C20-24 Alkyl Dimethicone, C24-28 Alkyl Dimethicone, C26-28 Alkyl Dimethicone, C30-45 Alkyl Dimethicone, C30-60 Alkyl Dimethicone, C32 Alkyl Dimethicone, C30-45 Alkyl Dimethicone/Polycyclohexene Oxide Crosspolymer, C26-28 Alkyldimethylsilyl Polypropylsilsesquioxane, C30-45 Alkyldimethylsilyl Polypropylsilsesquioxane, C20-24 Alkyl Methicone, C24-28 Alkyl Methicone, C26-28 Alkyl Methicone, C30-45 Alkyl Methicone, C20-28 Alkyl Perfluorodecylethoxy Dimethicone, C26-54 Alkyl Tetradecyl Dimethicone, Capryl Dimethicone, Caprylyl Dimethicone Ethoxy Glucoside, Caprylyl Methicone, Caprylyl Trimethicone, Carboxydecyl Trisiloxane, Castor Oil Bis-Hydroxypropyl Dimethicone Esters Cerotyl Dimethicone, Cetearyl Dimethicone Crosspolymer, Cetearyl Dimethicone/Vinyl Dimethicone Crosspolymer, Cetearyl Methicone, Cetrimonium Carboxydecyl PEG-8 Dimethicone, Cetrimonium Dimethicone PEG-7 Phthalate, Cetyl Behenyl Dimethicone, Cetyl Dimethicone, Cetyl Dimethicone/Bis-Vinyldimethicone Crosspolymer, Cetyl Hexacosyl Dimethicone, Cetyloxy Dimethicone, Cetyl PEG-8 Dimethicone, Cetyl PEG/PPG-15/15 Butyl Ether Dimethicone, Cetyl PEG/PPG-7/3 Dimethicone, Cetyl PEG/PPG-10/1 Dimethicone, Cetyl Triethylmonium Dimethicone PEG-8 Phthalate, Cetyl Triethylmonium Dimethicone PEG-8 Succinate, Copper Acetyl Tyrosinate Methylsilanol, Copper PCA Methylsilanol, C4-14 Perfluoroalkylethoxy Dimethicone, Cycloethoxymethicone, Cycloheptasiloxane, Cyclohexasiloxane, Cyclomethicone, Cyclopentasiloxane, Cyclophenylmethicone, Cyclotetrasiloxane, mCyclovinylmethicone, Cystine Bis-PG-Propyl Silanetriol, DEA PG-Propyl PEG/PPG-18/21 Dimethicone, Diisostearoyl Trimethylolpropane Siloxy Silicate, Dilauroyl Trimethylolpropane Siloxy Silicate, Dilinoleamidopropyl Dimethylamine Dimethicone PEG-7 Phosphate, Dimethicone, Dimethicone Crosspolymer, Dimethicone Crosspolymer-3, Dimethicone/Divinyldimethicone/Silsesquioxane Crosspolymer, Dimethicone Ethoxy Glucoside, Dimethicone Hydroxypropyl Trimonium Chloride, Dimethicone/Mercaptopropyl Methicone Copolymer, Dimethicone PEG-15 Acetate Dimethicone PEG-8 Adipate, Dimethicone PEG-7 Avocadoate, Dimethicone PEG-8 Avocadoate, Dimethicone PEG-8 Beeswax, Dimethicone PEG-8 Benzoate, Dimethicone PEG-8 Borageate, Dimethicone PEG-7 Cocoate, Dimethicone/PEG-10 Crosspolymer, Dimethicone/PEG-10/15 Crosspolymer, Dimethicone/PEG-15 Crosspolymer, Dimethicone PEG-7 Isostearate, Dimethicone PEG-8 Isostearate, Dimethicone PEG-7 Lactate, Dimethicone PEG-8 Lanolate, Dimethicone PEG-8 Laurate, Dimethicone PEG-8 Meadowfoamate, Dimethicone PEG-7 Octyldodecyl Citrate, Dimethicone PEG-7 Olivate, Dimethicone PEG8 Olivate, Dimethicone PEG-7 Phosphate, Dimethicone PEG-8 Phosphate, Dimethicone PEG-10 Phosphate, Dimethicone PEG-7 Phthalate, Dimethicone PEG-8 Phthalate, Dimethicone PEG-8 Polyacrylate, Dimethicone PEG/PPG-20/23 Benzoate, Dimethicone PEG/PPG-7/4 Phosphate, Dimethicone PEG/PPG-12/4 Phosphate, Dimethicone PEG-7 Succinate, Dimethicone PEG-8 Succinate, Dimethicone PEG-7 Sulfate, Dimethicone PEG-7 Undecylenate, Dimethicone PG-Diethylmonium Chloride, Dimethicone/Phenyl Vinyl Dimethicone Crosspolymer, Dimethicone/Polyglycerin-3 Crosspolymer, Dimethicone/PPG-20 Crosspolymer, Dimethicone Propylethylenediamine Behenate, Dimethicone Propyl PG-Betaine, Dimethicone/Silsesquioxane Copolymer, Dimethicone Silylate, Dimethicone?/inyl Dimethicone Crosspolymer, Dimethicone/Vinyltrimethylsiloxysilicate Crosspolymer, Dimethiconol, Dimethiconol Arginine, Dimethiconol Beeswax, Dimethiconol Behenate, Dimethiconol Borageate, Dimethiconol Candelillate, Dimethiconol Carnaubate, Dimethiconol Cysteine, Dimethiconol Dhupa Butterate, Dimethiconol Fluoroalcohol Dilinoleic Acid, Dimethiconol Hydroxystearate, Dimethiconol Illipe Butterate, Dimethiconol/IPDI Copolymer, Dimethiconol Isostearate, Dimethiconol Kokum Butterate, Dimethiconol Lactate, Dimethiconol Meadowfoamate, Dimethiconol Methionine, Dimethiconol/Methylsilanol/Silicate Crosspolymer, Dimethiconol Mohwa Butterate, Dimethiconol Panthenol, Dimethiconol Sal Butterate, Dimethiconol/Silica Crosspolymer, Dimethiconol/Silsesquioxane Copolymer, Dimethiconol Stearate, Dimethiconol/Stearyl, Methicone/Phenyl Trimethicone Copolymer, Dimethoxysilyl Ethylenediaminopropyl Dimethicone, Dimethylaminopropylamido PCA Dimethicone, Dimethyl Oxobenzo Dioxasilane, Dimethylsilanol Hyaluronate, Dioleyl Tocopheryl Methylsilanol, Diphenyl Amodimethicone, Diphenyl Dimethicone, Diphenyl Dimethicone Crosspolymer Diphenyl Dimethicone?/inyl Diphenyl Dimethicone/Silsesquioxane Crosspolymer, Diphenylethyl Benzyloxy Dilsiloxane, Diphenylisopropyl Dimethicone, Diphenylsiloxy Phenyl/Propyl Trimethicone, Diphenylsiloxy Phenyl Trimethicone Disiloxane, Disodium Amodimethicone Disuccinamide, Disodium PEG-12 Dimethicone Sulfosuccinate, Disodium PEG-8 Lauryl Dimethicone Sulfosuccinate, Divinyldimethicone/Dimethicone Copolymer, Divinyldimethicone/Dimethicone Crosspolymer, Drometrizole Trisiloxane, Ethylhexyl Acrylate/VP/Dimethicone Methacrylate Copolymer, Ethyl Methicone, Ethyl Trisiloxane, Fluoro C2-8 Alkyldimethicone, Gluconamidopropyl Aminopropyl Dimethicone, 4-(2-Beta-Glucopyranosiloxy) Propoxy-2-Hydroxybenzophenone, Glyceryl Undecyl Dimethicone, Glycidoxy Dimethicone, Hexadecyl Methicone, Hexyl Dimethicone, Hexyl Methicone, Hexyltrimethoxysilane, Hydrogen Dimethicone, Hydrogen Dimethicone/Octyl Silsesquioxane Copolymer, Hydrolyzed Collagen PG-Propyl Dimethiconol, Hydrolyzed Collagen PG-Propyl Methylsilanediol, Hydrolyzed Collagen PG-Propyl Silanetriol, Hydrolyzed Keratin PG-Propyl Methylsilanediol, Hydrolyzed Sesame Protein PG-Propyl Methylsilanediol, Hydrolyzed Silk PG-Propyl Methylsilanediol, Hydrolyzed Silk PG-Propyl Methylsilanediol Crosspolymer, Hydrolyzed Soy Protein/Dimethicone PEG-7 Acetate, Hydrolyzed Soy Protein PG-Propyl Methylsilanediol, Hydrolyzed Vegetable Protein PG-Propyl Silanetriol, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein PG-Propyl Methylsilanediol, Hydrolyzed Wheat Protein PG-Propyl Silanetriol, Hydroxyethyl Acetomonium PG-Dimethicone, Hydroxypropyldimethicone, Hydroxypropyl Dimethicone Behenate, Hydroxypropyl Dimethicone Isostearate, Hydroxypropyl Dimethicone Stearate, Isobutylmethacrylate/Bis-Hydroxypropyl Dimethicone Acrylate Copolymer, Isobutylmethacrylate/Trifluoroethylmethacrylate/Bis-Hydroxypropyl Dimethicone Acrylate Copolymer, Isopentyl Trimethoxycinnamate Trisiloxane, Isopolyglyceryl-3 Dimethicone, Isopolyglyceryl-3 Dimethiconol, Isopropyl Titanium Triisostearate/Triethoxysilylethyl, Polydimethylsiloxyethyl Dimethicone Crosspolymer, Isostearyl Carboxydecyl PEG-8 Dimethicone, Lactoyl Methylsilanol Elastinate, Lauryl Dimethicone, Lauryl Dimethicone PEG-15 Crosspolymer, Lauryl Dimethicone PEG-10 Phosphate, Lauryl Dimethicone/Polyglycerin-3 Crosspolymer, Lauryl Methicone, Lauryl PEG-8 Dimethicone, Lauryl PEG-10 Methyl Ether Dimethicone, Lauryl PEG-9 Polydimethylsiloxyethyl Dimethicone, Lauryl PEG/PPG-18/18 Methicone, Lauryl Phenylisopropyl Methicone, Lauryl Phenylpropyl Methicone, Lauryl Polydimethylsiloxyethyl Dimethicone/Bis-Vinyldimethicone Crosspolymer, Lauryl Polyglyceryl-3 Polydimethylsiloxyethyl Dimethicone, Lauryl Trimethicone, Linoleamidopropyl PG-Dimonium Chloride Phosphate Dimethicone, Methacryloyl Propyltrimethoxysilane, Methicone, Methoxy Amodimethicone/Silsesquioxane Copolymer, Methoxycinnamidopropyl Polysilsesquioxane, Methoxycinnamoylpropyl Silsesquioxane Silicate, Methoxy PEG-13 Ethyl Polysilsesquioxane, Methoxy PEG/PPG-7/3 Aminopropyl Dimethicone, Methoxy PEG/PPG-25/4 Dimethicone, Methoxy PEG-10 Propyltrimethoxysilane, Methyleugenyl PEG-8 Dimethicone, Methylpolysiloxane Emulsion, Methylsilanol Acetylmethionate, Methylsilanol Acetyltyrosine, Methylsilanol Ascorbate, Methylsilanol Carboxymethyl Theophylline, Methylsilanol Carboxymethyl Theophylline Alginate, Methylsilanol Elastinate, Methylsilanol Glycyrrhizinate, Methylsilanol Hydroxyproline, Methylsilanol Hydroxyproline Aspartate, Methylsilanol Mannuronate, Methylsilanol PCA, Methylsilanol PEG-7 Glyceryl Cocoate, Methylsilanol/Silicate Crosspolymer, Methylsilanol Spirulinate, Methylsilanol Tri-PEG-8 Glyceryl Cocoate, Methyl Trimethicone, Methyltrimethoxysilane, Myristylamidopropyl Dimethylamine Dimethicone PEG-7 Phosphate, Myristyl Methicone, Myristyl Trisiloxane, Nylon-611/Dimethicone Copolymer, PCA Dimethicone, PEG-7 Amodimethicone, PEG-8 Amodimethicone, PEG-8 Cetyl Dimethicone, PEG-3 Dimethicone, PEG-6 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG10 Dimethicone Crosspolymer, PEG-12 Dimethicone Crosspolymer, PEG-8 Dimethicone Dimer Dilinoleate, PEG-8 Dimethicone/Dimer Dilinoleic Acid Copolymer, PEG-10 Dimethicone/Vinyl Dimethicone Crosspolymer, PEG-8 Distearmonium Chloride PG-Dimethicone, PEG-10/Lauryl Dimethicone Crosspolymer, PEG-15/Lauryl Dimethicone Crosspolymer, PEG-15/Lauryl Polydimethylsiloxyethyl Dimethicone Crosspolymer, PEG-8 Methicone, PEG-6 Methicone Acetate, PEG-6 Methyl Ether Dimethicone, PEG-7 Methyl Ether Dimethicone, PEG-8 Methyl Ether Dimethicone, PEG-9 Methyl Ether Dimethicone, PEG-10 Methyl Ether Dimethicone, PEG-11 Methyl Ether Dimethicone, PEG-32 Methyl Ether Dimethicone, PEG-8 Methyl Ether Triethoxysilane, PEG-10 Nonafluorohexyl Dimethicone Copolymer, PEG-4 PEG-12 Dimethicone, PEG-8 PG-Coco-Glucoside Dimethicone, PEG-9 Polydimethylsiloxyethyl Dimethicone, PEG/PPG-20/22 Butyl Ether Dimethicone, PEG/PPG-22/22 Butyl Ether Dimethicone, PEG/PPG-23/23 Butyl Ether Dimethicone, PEG/PPG-24/18 Butyl Ether Dimethicone, PEG/PPG-27/9 Butyl Ether Dimethicone, PEG/PPG-3/10 Dimethicone, PEG/PPG-4/12 Dimethicone, PEG/PPG-6/4 Dimethicone, PEG/PPG-6/11 Dimethicone, PEG/PPG-8/14 Dimethicone, PEG/PPG-8/26 Dimethicone, PEG/PPG-10/2 Dimethicone, PEG/PPG-12/16 Dimethicone, PEG/PPG-12/18 Dimethicone, PEG/PPG-14/4 Dimethicone, PEG/PPG-15/5 Dimethicone, PEG/PPG-15/15 Dimethicone, PEG/PPG-16/2 Dimethicone, PEG/PPG-16/8 Dimethicone, PEG/PPG-17/18 Dimethicone, PEG/PPG-18/6 Dimethicone, PEG/PPG-18/12 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, PEG/PPG-30/10 Dimethicone, PEG/PPG-25/25 Dimethicone/Acrylates Copolymer, PEG/PPG-20/22 Methyl Ether Dimethicone, PEG/PPG-24/24 Methyl Ether Glycidoxy Dimethicone, PEG/PPG-10/3 Oleyl Ether Dimethicone, PEG/PPG-5/3 Trisiloxane, PEG-4 Trifluoropropyl Dimethicone Copolymer, PEG-8 Trifluoropropyl Dimethicone Copolymer, PEG-10 Trifluoropropyl Dimethicone Copolymer, PEG-8 Trisiloxane, Perfluorocaprylyl methoxysilylethyl Methicone, Perfluorononyl Dimethicone, Perfluorononyl Dimethicone/Methicone/Amodimethicone Crosspolymer, Perfluorononylethyl Carboxydecyl Behenyl Dimethicone, Perfluorononylethyl Carboxydecyl Hexacosyl Dimethicone, Perfluorononylethyl Carboxydecyl Lauryl/Behenyl Dimethicone, Perfluorononylethyl Carboxydecyl Lauryl Dimethicone, Perfluorononylethyl Carboxydecyl PEG-8 Dimethicone, Perfluorononylethyl Carboxydecyl PEG-10 Dimethicone, Perfluorononylethyl Dimethicone/Methicone Copolymer, Perfluorononylethyl PEG-8 Dimethicone, Perfluorononylethyl Stearyl Dimethicone, Perfluorooctylethyl/Diphenyl Dimethicone Copolymer, Perfluorooctylethyl Triethoxysilane, Perfluorooctylethyl Trimethoxysilane, Perfluorooctylethyl Trisiloxane, Perfluorooctyl Triethoxysilane, PG-Amodimethicone, Phenethyl Dimethicone, Phenethyl Disiloxane, Phenyl Dimethicone, Phenylisopropyl Dimethicone, Phenyl Methicone, Phenyl Methiconol, Phenylpropyldimethylsiloxysilicate, Phenylpropyl Ethyl Methicone, Phenyl Propyl Trimethicone, Phenyl Propyl Trimethicone/Diphenylmethicone, Phenyl Trimethicone, Platinum Divinyldisiloxane, Polyacrylate-6, Polydiethylsiloxane, Polydimethylsiloxyethyl Dimethicone/Bis-Vinyldimethicone Crosspolymer, Polydimethylsiloxyethyl Dimethicone/Methicone Copolymer, Polydimethylsiloxy PEG/PPG-24/19 Butyl Ether Silsesquioxane, Polydimethylsiloxy PPG-13 Butyl Ether Silsesquioxane, Polyglyceryl-3 Disiloxane Dimethicone, Polyglyceryl-3/Lauryl Polydimethylsiloxyethyl Dimethicone Crosspolymer, Polyglyceryl-3 Polydimethylsiloxyethyl Dimethicone, Poly(Glycol Adipate)/Bis-Hydroxyethoxypropyl Dimethicone Copolymer, Polymethylsilsesquioxane, Polymethylsilsesquioxane/Trimethylsiloxysilicate, Polyphenylsilsesquioxane, Polypropylsilsesquioxane, Polysilicone-1, Polysilicone-2, Polysilicone-3, Polysilicone-4, Polysilicone-5, Polysilicone-6, Polysilicone-7, Polysilicone-8, Polysilicone-9, Polysilicone-10, Polysilicone-11, Polysilicone-12, Polysilicone-13, Polysilicone-14, Polysilicone-15, Polysilicone-16, Polysilicone-17, Polysilicone-18, Polysilicone-19, Polysilicone-20, Polysilicone-21, Polysilicone-18 Cetyl Phosphate, Polysilicone-1 Crosspolymer, Polysilicone-18 Stearate, Polyurethane-10, Potassium Dimethicone PEG-7 Panthenyl Phosphate, Potassium Dimethicone PEG-7 Phosphate, PPG-12 Butyl Ether Dimethicone, PPG-2 Dimethicone, PPG-12 Dimethicone, PPG-27 Dimethicone, PPG-4 Oleth-10 Dimethicone, Propoxytetramethyl Piperidinyl Dimethicone, Propyl Trimethicone, Quaternium-80, Retinoxytrimethylsilane, Silanediol Salicylate, Silanetriol, Silanetriol Arginate, Silanetriol Glutamate, Silanetriol Lysinate, Silanetriol Melaninate, Silanetriol Trehalose Ether, Silica, Silica Dimethicone Silylate, Silica Dimethyl Silylate, Silica Silylate, Silicon Carbide, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, SiliconeQuaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Silicone Quaternium-22, Silicone Quaternium-24, Silicone Quaternium-25, Siloxanetriol Alginate, Siloxanetriol Phytate, Simethicone, Sodium Carboxydecyl PEG-8 Dimethicone, Sodium Dimethicone PEG-7 Acetyl Methyltaurate, Sodium Hyaluronate Dimethylsilanol, Sodium Lactate Methylsilanol, Sodium Mannuronate Methylsilanol, Sodium PCA Methylsilanol, Sodium PG-Propyldimethicone Thiosulfate Copolymer, Sodium PG-Propyl Thiosulfate Dimethicone, Sodium Propoxyhydroxypropyl Thiosulfate Silica, Sorbityl Silanediol, Soy Triethoxysilylpropyldimonium Chloride, Stearalkonium Dimethicone PEG-8 Phthalate, Stearamidopropyl Dimethicone, Steardimonium Hydroxypropyl Panthenyl PEG-7 Dimethicone Phosphate Chloride, Steardimonium Hydroxypropyl PEG-7 Dimethicone Phosphate Chloride, Stearoxy Dimethicone, Stearoxymethicone/Dimethicone Copolymer, Stearoxytrimethylsilane, Stearyl Aminopropyl Methicone, Stearyl Dimethicone, Stearyl/Lauryl Methacrylate Crosspolymer, Stearyl Methicone, Stearyl Triethoxysilanek, Stearyl Trimethicone, Styrene/Acrylates/Dimethicone Acrylate Crosspolymer, Styrene/Acrylates/Dimethicone Copolymer, TEA-Dimethicone PEG-7 Phosphate, Tetrabutoxypropyl Trisiloxane, Tetramethyl Hexaphenyl Tetrasiloxane, Tetramethyl Tetraphenyl Trisiloxane, Tocopheryloxypropyl Trisiloxane, Trideceth-9 PG-Amodimethicone, Triethoxycaprylylsilane, Triethoxysilylethyl Dimethicone/Methicone Copolymer, Triethoxysilylethyl Polydimethylsiloxyethyl Dimethicone, Triethoxysilylethyl Polydimethylsiloxyethyl Hexyl Dimethicone, Triethoxysilylpropylcarbamoyl Ethoxypropyl Butyl Dimethicone, Trifluoromethyl C1-4 Alkyl Dimethicone, Trifluoropropyl Cyclopentasiloxane, Trifluoropropyl Cyclotetrasiloxane, Trifluoropropyl Dimethicone, Trifluoropropyl Dimethicone/PEG-10 Crosspolymer, Trifluoropropyl Dimethicone/Trifluoropropyl Divinyldimethicone Crosspolymer, Trifluoropropyl Dimethicone/Vinyl Trifluoropropyl, Dimethicone/Silsesquioxane Crosspolymer, Trifluoropropyl Dimethiconol, Trifluoropropyldimethyl/trimethylsiloxysilicate, Trifluoropropyl Methicone, Trimethoxycaprylylsilane, Trimethoxysilyl Dimethicone, Trimethyl Pentaphenyl Trisiloxane, Trimethylsiloxyamodimethicone, Trimethylsiloxyphenyl Dimethicone, Trimethylsiloxysilicate, Trimethylsiloxysilicate/Dimethicone Crosspolymer, Trimethylsiloxysilicate/Dimethiconol Crosspolymer, Trimethylsiloxysilylcarbamoyl Pullulan, Trimethylsilyl Hydrolyzed Conchiolin Protein PG-Propyl Methylsilanediol Crosspolymer, Trimethylsilyl Hydrolyzed Silk PG-Propyl Methylsilanediol Crosspolymer, Trimethylsilyl Hydrolyzed Wheat Protein PG-Propyl Methylsilanediol Crosspolymer, Trimethylsilyl Pullulan, Trimethylsilyl Trimethylsiloxy Glycolate, Trimethylsilyl Trimethylsiloxy Lactate, Trimethylsilyl Trimethylsiloxy Salicylate, Triphenyl Trimethicone, Trisiloxane, Tris-Tributoxysiloxymethylsilane, Undecylcrylene Dimethicone, Vinyl Dimethicone, Vinyl Dimethicone/Lauryl Dimethicone Crosspolymer, Vinyl Dimethicone/Methicone Silsesquioxane Crosspolymer, Vinyldimethyl/Trimethylsiloxysilicate Stearyl Dimethicone Crosspolymer, VP/Dimethiconylacrylate/Polycarbamyl/Polyglycol Ester, Zinc Carboxydecyl Trisiloxane and Zinc Dimethicone PEG-8 Succinate and mixtures thereof.

More preferably the silicones to be contained in the mixture according to the inventions are Dimethicone, Cyclomethicone, Phenyl Trimethicone, Cyclohexasiloxane and Cyclopentasiloxane. A detailed overview of suitable volatile silicones can be found in Todd et al. in Cosm. Toil. 91, 27 (1976).

### Waxes and Stabilizers

Besides natural oils used, waxes may also be present in the preparations, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Metal salts of fatty acids such as, for example, magnesium, aluminium and/or zinc stearate or ricinoleate may be used as stabilizers.

### Primary Sun Protection Filters

Primary sun protection filters in the context of the invention are, for example, organic substances (light filters) which are liquid or crystalline at room temperature and which are capable of absorbing ultraviolet radiation and of releasing the energy absorbed in the form of longer-wave radiation, for example heat.

The formulations according to the invention advantageously contain at least one UV-A filter and/or at least one UV-B filter and/or a broadband filter and/or at least one inorganic pigment. Formulations according to the invention preferably contain at least one UV-B filter or a broadband filter, more particularly preferably at least one UV-A filter and at least one UV-B filter.

The UV filters cited below which can be used within the context of the present invention are preferred but naturally are not limiting. UV filters which are preferably used are selected from the group consisting of one, two, three, four, five or more of the following species:

| | | SUP- |
|---|---|---|
| UV FILTER | TRADEMARK | PLIER |
| 4-Methylbenzylidene Camphor | Neo Heliopan® MBC | Symrise |
| Anthranilic Acid Menthyl Ester | Neo Heliopan® MA | Symrise |
| Benzophenone-3 | Neo Heliopan® BB | Symrise |
| Benzophenone-4 | Uvinul® MS40 | BASF |
| Bis-Ethylhexyloxyphenol | Neo Heliopan® BMT | Symrise |
| Methoxyphenyl | | |
| Triazine | | |
| Butyl Methoxydibenzoylmethane | Neo Heliopan® 357 | Symrise |
| Diethylamine Hydroxybenzoyl | Uvinul® A Plus | BASF |
| Hexyl Benzoate | | |
| Diethylhexyl Butylamido Triazone | Iscotrizinol | 3V |
| Disodium Phenyl Dibenimidazole | Neo Heliopan® AP | Symrise |
| Sulfonic Acid | | |
| Ethylhexyl Methoxycinnamate | Neo Heliopan® AV | Symrise |
| Ethylhexyl salicylate | Neo Heliopan® OS | Symrise |
| Ethylhexyl triazone | Uvinul® T150 | BASF |
| Homoslate | Neo Heliopan® HMS | Symrise |
| Isoamyl p-Methoxycinnamate | Neo Heliopan® E | Symrise |
| Methoxypropylamine | 1000 | BASF |
| Cyclohexenylidene | | |
| Ethoxyethylcyanoacetate | | |
| Methylene Bis-Benzotriazolyl | Tinosorb® M | BASF |
| Tetramethylbutylphhenol | | |
| (nano) | | |
| Octocrylene | Neo Heliopan® 303 | Symrise |
| Phenylbenzimidazole Sulfonic Acid | Neo Heliopan® Hydro | Symrise |
| Phenylene Bis-Diphenyltriazone | Triasorb | Pierre |
| | | Fabre |
| Polysilicone-15 | Parsol® SLX | DSM |
| Tris-Biphenyl Triazine (nano) | Tinosorb® A2B | BASF |

In a preferred embodiment the sun protection filter forming component (ii) represents a blend of UV-A- and UV-B-filters selected from the group consisting of homosalate, octocrylene, bis-ethylhexyloxyphenol methoxyphenyl triazine, butyl methoxydibenzoylmethane, ethylhexyl salicylate and mixtures thereof. Particular preferred is a blend of all these filters which is commercially available in the market under the trademark NeoHeliopan® Flat (SYMRISE), which also subject to WO 2020 088778 A1.

Suitable pigments encompass oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures thereof.

In a further preferred embodiment a formulation according to the invention contains a total amount of sunscreen agents, i.e. in particular UV filters and/or inorganic pigments (UV filtering pigments) so that the formulation according to the invention has a light protection factor of greater than or equal to 5 and up to 50. Such formulations according to the invention are particularly suitable for protecting the skin and hair.

### Secondary Sun Protection Filters

Besides the groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Advantageous inorganic secondary light protection pigments are finely dispersed metal oxides and metal salts which are also mentioned in WO 2005 123101 A1. The total quantity of inorganic pigments, in particular hydrophobic inorganic micro-pigments in the finished cosmetic preparation according to the present invention is advantageously from 0.1 to 30% by weight, preferably 0.5 to 10.0% by weight, in each case based on the total weight of the preparation.

Also preferred are particulate UV filters or inorganic pigments, which can optionally be hydrophobed, can be used, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures thereof.

### Biogenic Agents and Antioxidants

Biogenic active substances include, for example, tocopherol, tocopherol acetate, tocopherol palmitate, ascorbic acid, (deoxy)ribonucleic acid and its fragmentation products, β-glucans, retinol, bisabolol, allantoin, phytantriol, panthenol, AHA acids, amino acids, ceramides, pseudoceramides, essential oils, plant extracts, such as such as prunus extract, bambaranus extract and vitamin complexes.

Antioxidants interrupt the photochemical reaction chain which is triggered when UV radiation penetrates the skin. Typical examples are amino acids (e.g. glycine, histidine, tyrosine, tryptophan) and their derivatives, imidazoles (e.g. urocanic acid) and their derivatives, peptides like D,L-carnosine, D-carnosine, L-carnosine and their derivatives (e.g. anserine), carotenoids, carotenes (e.g. -carotene, lycopene) and their derivates, chlorogenic acid and its derivatives, lipoic acid and its derivatives (e.g. dihydrolic acid), aurothioglucose, propylthiouracil and other thiols (e.g. thioredoxin, glutathione, cysteine, cystine, cystamin and their glycosyl, N-acetyl, methyl, ethyl, propyl, amyl, butyl and lauryl, palmitoyl, oleyl, linoleyl, cholesteryl and glyceryl esters) and their salts Dilaurylthiodipropionate, ditearylthiodipropionate, thiodipropionic acid and its derivatives (esters, ethers, peptides, lipids, nucleotides, nucleosides and salts) as well as sulfoximine compounds (e.g. (e.g. buthionine sulfoximines, homocysteine sulfoximines, butionine sulfones, penta-, hexa-, heptathionine sulfoximines) in very low tolerated dosages (e.g. pmol to mol/kg), furthermore (metal) chelators (e.g. hydroxy fatty acids, palmitic acid, phytinic acid, lactoferrin), hydroxy acids (e.g. (e.g. citric acid, lactic acid, malic acid), humic acid, gallic acid, bile extracts, bilirubin, biliverdin, EDTA, EGTA and their derivatives, unsaturated fatty acids and their derivatives (e.g. linolenic acid, linoleic acid, oleic acid), folic acid and its derivatives, ubiquinone and ubiquinol and their derivatives, vitamin C and its derivatives (e.g. ascorbyl palmitate, Mg-ascorbyl phosphate, ascorbylacetate), tocopherols and derivatives (e.g. vitamin E acetate), vitamin A and derivates (vitamin A palmitate) as well as conifer aryl benzoate of benzoic resin, rutinic acid and its derivatives, glycosylrutin, ferulic acid, furfurylidene glucitol, carnosine, butyl hydroxytoluene, butylhydroxyanisole, nordihydroguaiac resin acid, nordihydroguajaretic acid, trihydroxybutyrophenone, uric acid and its derivatives, mannose and its derivatives, superoxide dismutase, zinc and its derivatives (e.g. e.g. ZnO, ZnSO4) selenium and its derivatives (e.g. selenium-methionine), stilbenes and their derivatives (e.g. styrene oxide, trans-stilbene oxide) and the derivatives suitable for the invention (salts, esters, ethers, sugars, nucleotides, nucleosides, peptides and lipids) of these named active substances.

### Actives Modulating Hair Pigmentation

Preferred active ingredients for hair lightening are selected from the group consisting of: kojic acid (5-hydroxy-2-hydroxymethyl-4-pyranone), kojic acid derivatives, preferably kojic acid dipalmitate, arbutin, ascorbic acid, ascorbic acid derivatives, preferably magnesium ascorbyl phosphate, hydroquinone, hydroquinone derivatives, resorcinol, resorcinol derivatives, preferably 4-alkylresorcinols and 4-(1-phenylethyl)1,3-dihydroxybenzene (phenylethyl resorcinol), cyclohexylcarbamates (preferably one or more cyclohexyl carbamates disclosed in WO 2010/122178 and WO 2010/097480), sulfur-containing molecules, preferably glutathione or cysteine, alpha-hydroxy acids (preferably citric acid, lactic acid, malic acid), salts and esters thereof, N-acetyl tyrosine and derivatives, undecenoyl phenylalanine, gluconic acid, chromone derivatives, preferably aloesin, flavonoids, 1-aminoethyl phosphinic acid, thiourea derivatives, ellagic acid, nicotinamide (niacinamide), zinc salts, preferably zinc chloride or zinc gluconate, thujaplicin and derivatives, triterpenes, preferably maslinic acid, sterols, preferably ergosterol, benzofuranones, preferably senkyunolide, vinyl guiacol, ethyl guiacol, dionic acids, preferably octodecene dionic acid and/or azelaic acid, inhibitors of nitrogen oxide synthesis, preferably L-nitroarginine and derivatives thereof, 2,7-dinitroindazole or thiocitrulline, metal chelators (preferably alpha-hydroxy fatty acids, phytic acid, humic acid, bile acid, bile extracts, EDTA, EGTA and derivatives thereof), retinoids, soy milk and extract, serine protease inhibitors or lipoic acid or other synthetic or natural active ingredients for skin and hair lightening, the latter preferably used in the form of an extract from plants, preferably bearberry extract, rice extract, papaya extract, turmeric extract, mulberry extract, bengkoang extract, nutgrass extract, liquorice root extract or constituents concentrated or isolated therefrom, preferably glabridin or licochalcone A, artocarpus extract, extract of Rumex and Ramulus species, extracts of pine species (Pinus), extracts of Vitis species or stilbene derivatives isolated or concentrated therefrom, saxifrage extract, scutelleria extract, grape extract and/or microalgae extract, in particular Tetraselmis suecica Extract.

Advantageous skin and hair tanning active ingredients in this respect are substrates or substrate analogues of tyrosinase such as L-tyrosine, N-acetyl tyrosine, L-DOPA or L-dihydroxyphenylalanine, xanthine alkaloids such as caffeine, theobromine and theophyl-line and derivatives thereof, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)2), manganese(ll) bicarbonate complexes (“pseudocat-alases”) as described for example in EP 0 584 178, tetrasubstituted cyclohexene deriva-tives as described for example in WO 2005/032501, isoprenoids as described in WO 2005/102252 and in WO 2006/010661, melanin derivatives such as Melasyn-100 and MelanZe, diacyl glycerols, aliphatic or cyclic diols, psoralens, prostaglandins and ana-logues thereof, activators of adenylate cyclase and compounds which activate the transfer of melanosomes to keratinocytes such as serine proteases or agonists of the PAR-2 receptor, extracts of plants and plant parts of the Chrysanthemum species, san-guisorba species, walnut extracts, urucum extracts, rhubarb extracts, microalgae extracts, in particular Isochrysis galbana, trehalose, erythru-lose and dihydroxyacetone. Flavonoids which bring about skin and hair tinting or browning (e.g. quercetin, rhamnetin, kaempferol, fisetin, genistein, daidzein, chrysin and api-genin, epicatechin, diosmin and diosmetin, morin, quercitrin, naringenin, hesperidin, phloridzin and phloretin) can also be used.

The amount of the aforementioned examples of additional active ingredients for the modulation of skin and hair pigmentation (one or more compounds) in the products according to the invention is then preferably 0.00001 to 30 wt. %, preferably 0.0001 to 20 wt. %, particularly preferably 0.001 to 5 wt. %, based on the total weight of the preparation.

### Hair Growth Activators or Inhibitors

Formulations and products according to the present invention may also comprise one or more hair growth activators, i.e. agents to stimulate hair growth. Hair growth activators are preferably selected from the group consisting of pyrimidine derivatives such as 2,4-diaminopyrimidine-3-oxide (Aminexil), 2,4-diamino-6-piperidinopyrimidine-3-oxide (Minoxidil) and derivatives thereof, 6-amino-1,2-dihydro-1-hydroxy-2-imino-4-piperidinopyrimidine and its derivatives, xanthine alkaloids such as caffeine, theobromine and theophylline and derivatives thereof, quercetin and derivatives, dihydroquercetin (taxifolin) and derivatives, potassium channel openers, antiandrogenic agents, synthetic or natural 5-reductase inhibitors, nicotinic acid esters such as tocopheryl nicotinate, benzyl nicotinate and C1-C6 alkyl nicotinate, proteins such as for example the tripeptide Lys-Pro-Val, diphencypren, hormons, finasteride, dutasteride, flutamide, bicalutamide, pregnane derivatives, progesterone and its derivatives, cyproterone acetate, spironolactone and other diuretics, calcineurin inhibitors such as FK506 (Tacrolimus, Fujimycin) and its derivatives, Cyclosporin A and derivatives thereof, zinc and zinc salts, polyphenols, procyanidins, proanthocyanidins, phytosterols such as for example beta-sitosterol, biotin, eugenol, (±)-betacitronellol, panthenol, glycogen for example from mussels, extracts from microorganisms, algae, plants and plant parts of for example the genera dandelion (Leontodon or Taraxacum), Orthosiphon, Vitex, Coffea, Paullinia, Theobroma, Asiasarum, Cucurbita or Styphnolobium, Serenoa repens (saw palmetto), Sophora flavescens, Pygeum africanum, Panicum miliaceum, Cimicifuga racemosa, Glycine max, Eugenia caryophyllata, Cotinus coggygria, Hibiscus rosa-sinensis, Camellia sinensis, Ilex paraguariensis, Isochrysis galbana, licorice, grape, apple, barley or hops or/nd hydrolysates from rice or wheat.

Alternatively, formulations and products according to the present invention may comprise one or more hair growth inhibitors (as described above), i.e. agents to reduce or prevent hair growth. Hair growth inhibitors are preferably selected from the group consisting of activin, activin derivatives or activin agonists, ornithine decarboxylase inhibitors such as alpha-difluoromethylornithine or pentacyclic triterpenes like for example ursolic acid, betulin, betulinic acid, oleanolic acid and derivatives thereof, 5alpha-reductase inhibitors, androgen receptor antagonists, S-adenosylmethionine decarboxylase inhibitors, gamma-glutamyl transpeptidase inhibitors, transglutaminase inhibitors, soybean-derived serine protease inhibitors, extracts from microorganisms, algae, different microalgae or plants and plant parts of for example the families Leguminosae, Solanaceae, Graminae, Asclepiadaceae or Cucurbitaceae, the genera Chondrus, Gloiopeltis, Ceramium, Durvillea, Glycine max, Sanguisorba officinalis, Calendula officinalis, Hamamelis virginiana, Arnica montana, Salix alba, Hypericum perforatum or Gymnema sylvestre.

### Anti-Inflammatory Agents

Suitable anti-inflammatory agents may be selected from the group formed by:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

### Anti-Microbial Agents

Suitable anti-microbial agents are, in principle, all substances effective against Gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chloro-phenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

### Enzyme Inhibitors

Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen CAT). The substances inhibit enzyme activity, thereby reducing the formation of odour. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

### Odour Absorbers and Antiperspirant Active Agents

Suitable odour absorbers are substances which are able to absorb and largely retain odour-forming compounds. They lower the partial pressure of the individual components, thus also reducing their rate of diffusion. It is important that perfumes must remain unimpaired in this process. Odour absorbers are not effective against bacteria. They comprise, for example, as main constituent, a complex zinc salt of ricinoleic acid or specific, largely odour-neutral fragrances which are known to the person skilled in the art as “fixatives”, such as, for example, extracts of labdanum or styrax or certain abietic acid derivatives. The odour masking agents are fragrances or perfume oils, which, in addition to their function as odour masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal products, such as, for example, civet and castoreum. Typical synthetic fragrance compounds are products of the ester, ether, aldehyde, ketone, alcohol, and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linaool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable astringent antiperspirant active ingredients are primarily salts of aluminium, zirconium or of zinc. Such suitable antihydrotic active ingredients are, for example, aluminium chloride, aluminium chlorohydrate, aluminium dichlorohydrate, aluminium sesquichlorohydrate and complex compounds thereof, e.g. with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, e.g. with amino acids, such as glycine.

### Film Formers and Anti-Dandruff Agents

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

### Carriers and Hydrotropes

Preferred cosmetics carrier materials are solid or liquid at 25° C. and 1013 mbar (including highly viscous substances) as for example glycerol, 1,2-propylene glycol, 1,2-butylene glycol, 1,3-propylene glycol, 1,3-butylene glycol, ethanol, water and mixtures of two or more of said liquid carrier materials with water. Optionally, these preparations according to the invention may be produced using preservatives or solubilizers. Other preferred liquid carrier substances, which may be a component of a preparation according to the invention are selected from the group consisting of oils such as vegetable oil, neutral oil and mineral oil.

Preferred solid carrier materials, which may be a component of a preparation according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin and inulin and mixtures of two or more of these solids, in particular maltodextrins (preferably with a dextrose equivalent value of 15-20), lactose, silicon dioxide and/or glucose.

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives and/or Product Protection Agents

Suitable preservatives are, for example, phenoxyethanol, sodium benzoate or sorbic acid, blends of the mentioned ingredients and the other classes of compounds listed in Appendix 6, Parts A and B of the Kosmetikverordnung (“Cosmetics Directive”). Alternative products which could improve the product protection are for example 1,2-alkanediols such as for example 1,2-penatnediol, 1,2-hexanediol, 1,2-octanediol, 1,2-decanediol, 1,2-dodecanediol and mixtures thereof, 4-hydroxy acetophenone.

### Dyes

Suitable dyes are any of the substances suitable and approved for cosmetic purposes as listed, for example, in the publication “Kosmetische Färbemittel” of the Farbstoffkommission der Deutschen Forschungsgemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. Advantageous coloured pigments are for example titanium dioxide, mica, iron oxides (e.g. Fe₂O₃ Fe₃O4, FeO(OH)) and/or tin oxide. Advantageous dyes are for example carmine, Berlin blue, chromium oxide green, ultramarine blue and/or manganese violet.

### Cosmetic Preparations

Preferred compositions according to the present inventions are selected from the group of products for treatment, protecting, care and cleansing of the skin and/or hair or as a make-up product, preferably as a leave-on product (meaning that the one or more compounds stay on the skin and/or hair for a longer period of time, compared to rinse-off products).

The formulations according to the invention are preferably in the form of an emulsion, e.g. W/O (water-in-oil), O/W (oil-in-water), W/O/W (water-in-oil-in-water), O/W/O (oil-in-water-in-oil) emulsion, PIT emulsion, Pickering emulsion, emulsion with a low oil content, micro- or nanoemulsion, a solution, e.g. in oil (fatty oils or fatty acid esters, in particular C₆-C₃₂ fatty acid C₂-C₃₀ esters) or silicone oil, dispersion, suspension, creme, lotion or milk, depending on the production method and ingredients, a gel (including hydrogel, hydrodispersion gel, oleogel), spray (e.g. pump spray or spray with propellant) or a foam or an impregnating solution for cosmetic wipes, a detergent, e.g. soap, synthetic detergent, liquid washing, shower and bath preparation, bath product (capsule, oil, tablet, salt, bath salt, soap, etc.), effervescent preparation, a skin care product such as e.g. an emulsion (as described above), ointment, paste, gel (as described above), oil, balsam, serum, powder (e.g. face powder, body powder), eau de perfume, eau de toilette, after-shave, a mask, a pencil, stick, roll-on, pump, aerosol (foaming, non-foaming or post-foaming), a deodorant and/or antiperspirant, mouthwash and mouth rinse, a foot care product (including keratolytic, deodorant), an insect repellent, a sunscreen, aftersun preparation, a shaving product, aftershave balm, pre- and aftershave lotion, a depilatory agent, a hair care product such as e.g. shampoo (including 2-in-1 shampoo, anti-dandruff shampoo, baby shampoo, shampoo for dry scalps, concentrated shampoo), conditioner, hair tonic, hair water, hair rinse, styling creme, pomade, perm and setting lotion, hair spray, styling aid (e.g. gel or wax), hair smoothing agent (detangling agent, relaxer), hair dye such as e.g. temporary direct-dyeing hair dye, semi-permanent hair dye, permanent hair dye, hair conditioner, hair mousse, eye care product, make-up, make-up remover or baby product.

Auxiliary substances and additives can be included in quantities of 5 to 99% b.w., preferably 10 to 80% b.w., based on the total weight of the formulation. The amounts of cosmetic or dermatological auxiliary agents and additives and perfume to be used in each case can easily be determined by the person skilled in the art by simple trial and error, depending on the nature of the particular product.

The preparations can also contain water in a quantity of up to 99 wt.-percent., preferably from about 5 to about 80 wt.-percent and more preferably either from about 10 to about 50 or from about 60 to about 80 wt.-percent based on the total weight of the preparation.

### Oral Compositions

Finally, another object of the present invention is directed to a preferably non-therapeutic oral and/or oral care composition. Typical examples for suitable oral compositions encompass (hard boiled) candies, compressed tablets, chewing gums, toothpastes and mouth washes. The manufacture and composition of said oral compositions are described as follows:

### Candies

According to the present invention the preferred candies are so-called hard-boiled candies. Their bases are usually prepared from a mixture of sugar and other carbohydrates that are kept in an amorphous or glassy condition. This form can be considered a solid syrup of sugars generally having up to about 4.5% b.w. moisture, based on the weight of the candy base, with about 0.5 to about 2.5% b.w. being preferred and about 1.0 to about 1.5% b.w. being most preferred. Such materials normally contain up to 65% b.w. corn syrup, up to 80% b.w. sugar and from 0.1 to 5.0% b.w. water. Generally, the ratio of sugar (or other sweetener suitable for candy formulation) to corn syrup is within the range of about 70:25 to about 45:55 with about 60:40 being preferred. The syrup component generally is prepared from corn syrups high in fructose, but may include other materials. Further ingredients such as flavourings, sweeteners, acidulents, colorants and so forth may also be added.

Hard boiled candy bases may also be prepared from non-fermentable sugars such as sorbitol, mannitol, xylitol, maltitol, hydrogenated starch hydrolysate, hydrogenated corn syrup and mixtures thereof. The candy bases may contain up to about 95% sorbitol, a mixture of sorbitol and mannitol at a ratio of about 9.5 to 0.5 up to about 7.5 to 2.5 and hydrogenated corn syrup up to about 55% of the syrup component.

### Compressed Tablets

According to the present invention the oral compositions can represent compressed tablets, comprising the liquid flavour in amounts of typically about 0.1 to about 0.6% b.w. and preferably about 0.5% b.w.

### Chewing Gums

Chewing gums typically consist of a water-insoluble vase component, a water-soluble component and additives providing for example a specific flavour.

The water-insoluble base, which is also known as the “gum base”, typically comprises natural or synthetic elastomers, resins, fats and oils, plasticizers, fillers, softeners, dyes and optionally waxes. The base normally makes up 5 to 95% by weight, preferably 10 to 50% by weight and more particularly 20 to 35% by weight of the composition as a whole. In one typical embodiment of the invention, the base consists of 20 to 60% by weight synthetic elastomers, 0 to 30% by weight natural elastomers, 5 to 55% by weight plasticizers, 4 to 35% by weight fillers, 5 to 35% by weight softeners and small amounts of additives, such as dyes, antioxidants and the like, with the proviso that they are soluble in water at best in small quantities.

Suitable synthetic elastomers are, for example, polyisobutylenes with average molecular weights (as measured by GPC) of 10,000 to 100,000 and preferably 50,000 to 80,000, isobutylene/isoprene copolymers (“butyl elastomers”), styrene/butadiene copolymers (styrene:butadiene ratio, for example, 1:3 to 3:1). polyvinyl acetates with average molecular weights (as measured by GPC) of 2,000 to 90,000 and preferably 10,000 to 65,000, polyisoprenes, poly-ethylenes, vinyl acetate/vinyl laurate copolymers and mixtures thereof. Examples of suitable natural elastomers are rubbers, such as for example smoked or liquid latex or guayuls, and natural gums, such as jelutong, lechi caspi, perillo, sorva, massaranduba balata, massaranduba chocolate, nispero, rosindinba, chicle, gutta hang kang and mixtures thereof. The choice of the synthetic and natural elastomers and their mixing ratios essentially depends on whether or not bubbles are to be produced with the chewing gums (bubble gums). Elastomer mixtures containing jelutong, chicle, sorva and massaranduba are preferably used.

In most cases, the elastomers are too hard or lack plasticity for satisfactory processing, so that it has been found to be of advantage to use special plasticizers which, of course, must also satisfy in particular all requirements relating to acceptability as food additives. In this respect, suitable plasticizers are, above all, esters of resin acids, for example esters of lower aliphatic alcohols or polyols with completely or partly hydrogenated, monomeric or oligomeric resin acids. In particular, the methyl, glycerol or pentaerythritol esters or mixtures thereof are used for this purpose. Alternatively, terpene resins, which may be derived from .alpha.-pinene, .beta.-pinene, .delta.-limonene or mixtures thereof, could also be used.

Suitable fillers or texturizers are magnesium or calcium carbonate, ground pumice stone, silicates, especially magnesium or aluminium silicates, clays, aluminium oxides, talcum, titanium dioxide, mono-, di- and tricalcium phosphate and cellulose polymers.

Suitable softeners or emulsifiers are tallow, hydrogenated tallow, hydrogenated or partly hydrogenated vegetable oils, cocoa butter, partial glycerides, lecithin, triacetin and saturated or unsaturated fatty acids containing 6 to 22 and preferably 12 to 18 carbon atoms and mixtures thereof.

Suitable dyes and whiteners are, for example, the FD&C types, plant and fruit extracts permitted for colouring foods and titanium dioxide. The gum bases may also contain waxes or may be wax-free

In addition to the water-insoluble gum base, chewing gum preparations regularly contain a water-soluble component which is formed, for example, by softeners, sweeteners, fillers, flavours, flavour enhancers, emulsifiers, dyes, acidifiers, antioxidants and the like, with the proviso that the constituents have at least adequate solubility in water. Accordingly, individual constituents may belong both to the water-insoluble phase and to the water-soluble phase, depending on the water solubility of the special representatives. However, combinations may also be used, for example a combination of a water-soluble and a water-insoluble emulsifier, in which case the individual representatives are present in different phases. The water-insoluble component usually makes up 5 to 95% by weight and preferably 20 to 80% by weight of the preparation.

Water-soluble softeners or plasticizers are added to the chewing gum compositions to improve chewability and the chewing feel and are present in the mixtures in quantities of typically 0.5 to 15% by weight. Typical examples are glycerol, lecithin and aqueous solutions of sorbitol, hydrogenated starch hydrolysates or corn syrup.

Fillers are particularly suitable for the production of low-calorie chewing gums and may be selected, for example, from polydextrose, raftilose, raftilin, fructo-oligosaccharides (NutraFlora), palatinose oligosaccharides, guar gum hydrolyzates (Sun Fiber) and dextrins.

The chewing gums may additionally contain auxiliaries and additives which are suitable, for example, for dental care, more particularly for controlling plaque and gingivitis, such as for example chlorhexidine, CPC or triclosan. They may also contain pH adjusters (for example buffer or urea), anticaries agents (for example phosphates or fluorides), biogenic agents (antibodies, enzymes, caffeine, plant extracts), providing these substances are permitted in foods and do not undesirably interact with one another.

### Toothpastes and Mouthwashes

Toothpastes or tooth creams are generally understood to be paste-like preparations of water, thickeners, humectants, abrasives or polishes, surfactants, sweeteners, flavorings, deodorizing agents and agents active against oral and dental diseases. In toothpastes according to the invention, any of the usual polishes may be used, such as chalk, dicalcium phosphate, insoluble sodium metaphosphate, aluminium silicates, calcium pyrophosphate, finely particulate synthetic resins, silicas, aluminium oxide and aluminium oxide trihydrate. Particularly suitable polishes for toothpastes according to the invention are finely particulate xerogel silicas, hydrogel silicas, precipitated silicas, aluminium oxide trihydrate and finely particulate .alpha.-alumina, or mixtures of these polishes. Such polishes are preferably used in quantities of from about 15 to 40% by weight of the toothpaste. Preferred humectants used for toothpastes according to the invention include low molecular weight polyethylene glycols, glycerol, sorbitol or mixtures thereof in quantities of up to about 50% by weight of the toothpaste. Among the known thickeners for use with toothpastes according to the invention, particularly preferred are the thickening, finely particulate gel silicas and nonionic hydrocolloids, such as hydroxy ethyl cellulose, hydroxy propyl guar, hydroxy ethyl starch, polyvinyl pyrrolidone, high molecular weight polyethylene glycol and vegetable gums, such as tragacanth, agaragar, carrageen moss, gum arabic and xanthan gum. The desired flavor and aroma for preparations in accordance with the invention may be obtained by adding the components (a) and/or (b) and optionally also (c). It is also advantageous adding caries inhibitors to the oral preparations in the form of, for example, alkali fluorides, alkali monofluorophosphates or alkali salts of organophosphonic acids. In addition, the oral preparations according to the invention may contain other standard auxiliaries, such as dyes, preservatives and opacifiers, for example titanium dioxide. For mouthwashes, the oral compositions according to the invention may readily be combined with aqueous-alcoholic solutions containing different amounts of ethereal oils, emulsifiers, astringent and toning drug extracts, caries-inhibiting additives and flavour correctants.

### Additives

The oral compositions of the present invention may include additional additives as for examples sweeteners or vitamins, in amounts of from about 0.1 to about 10% b.w. These additives may also represent components of the respective medicaments.

### Sweeteners

Suitable sweet-tasting substances, including natural sources of these substances (component e5), such as for example sweet-tasting carbohydrates or sugars (e.g. sucrose (synonymous with saccharose), trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin) or vegetable preparations containing predominantly these carbohydrates (e.g. from sugar beet (Beta vulgaris ssp., sugar fractions, sugar syrup, molasses), from sugar cane (Saccharum officinarum ssp., e.g. molasses, sugar syrups), from sugar maple (Acer ssp.), from agave (agave thick juice), synthetic/enzymatic hydrolysates of starch or sucrose (e.g. invert sugar syrup, highly enriched fructose syrups made from corn starch), fruit concentrates (e.g. from apples or pears, apple syrup, pear syrup), sugar alcohols (e.g. erythritol, threitol, arabitol, ribitol, xylitol, sorbitol, mannitol, dulcitol, lactitol), proteins (e.g. miraculin, monellin, thaumatin, curculin, brazzein), sweeteners (magap, sodiumcyclamate, acesulfame K, neohesperidin dihydrochalcone, saccharin sodium salt, aspartame®, superaspartame, neotame, alitame, sucralose, stevioside, rebaudioside, lugduname, carrelame, sucrononate, sucrooctate, monatin, phyllodulcin), certain sweet-tasting amino acids (glycine, D-leucine, D-threonine, D-asparagine, D-phenylalanine, D-tryptophan, L-proline), other sweet-tasting low-molecular substances (e.g. hernandulcin, dihydrochalcone glycosides, glycyrrhizin, glycyrrhetinic acid ammonium salt or other glycyrrhetinic acid derivatives), liquorice extracts (Glycyrrhizza glabra ssp.), Lippia dulcis extracts, Momordica ssp. extracts or individual substances (in particular Momordica grosvenori [Luo Han Guo] and the mogrosides obtained therefrom), Hydrangea dulcis or Stevia ssp. (e.g. Stevia rebaudiana) extracts or individual substances.

### Vitamins

In another embodiment of the present invention the compositions may include vitamins (component el). Vitamins have diverse biochemical functions. Some have hormone-like functions as regulators of mineral metabolism (e.g., vitamin D), or regulators of cell and tissue growth and differentiation (e.g., some forms of vitamin A). Others function as antioxidants (e.g., vitamin E; and sometimes vitamin C). The largest numbers of vitamins (e.g. B complex vitamins) act as precursors for enzyme cofactors that help enzymes in their work as catalysts in metabolism. In this role, vitamins may be tightly bound to enzymes as part of prosthetic groups: For example, biotin is part of enzymes involved in making fatty acids. Vitamins may also be less tightly bound to enzyme catalysts as coenzymes, detachable molecules that function to carry chemical groups or electrons between molecules. For example, folic acid carries various forms of carbon group—methyl, formyl, and methylene—in the cell. Although these roles in assisting enzyme-substrate reactions are vitamins' best-known function, the other vitamin functions are equally important. In the course of the present invention suitable vitamins are selected from the group consisting of
Vitamin A (retinol, retinal, beta carotene),
Vitamin B₁ (thiamine),
Vitamin B₂ (riboflavin),
Vitamin B₃ (niacin, niacinamide),
Vitamin B₅ (panthothenic acid),
Vitamin B₆ (pyridoxine, pyridoxamine, paridoxal),
Vitamin B₇ (biotin),
Vitamin B₉ (folic acid, folinic acid),
Vitamin B₁₂ (cyanobalamin, hydoxycobalmin, methylcobalmin),
Vitamin C (ascorbic acid),
Vitamin D (cholecalciferol),
Vitamin E (tocopherols, tocotrienols), and
Vitamin K (phyolloquinone, menaquinone).

The preferred vitamins are ascorbic acid and tocopherols. Said vitamins may be present in the food composition in amounts of about 0.1 to about 5% b.w., and preferably about 0.5 to about 1% b.w.

### Food Compositions

Food compositions according to the invention are any preparations or compositions comprising the compositions according to the invention which are suitable for consumption and are used for nutrition or enjoyment purposes, and are generally products which are intended to be introduced into the human or animal oral cavity, to remain there for a certain time and then either be eaten (e.g. ready-to-eat foodstuffs or feeds, see also herein below) or removed from the oral cavity again (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the invention also include substances which in the unchanged, treated or prepared state are to be swallowed by a human or animal and then digested; in this respect, the orally consumable products according to the invention also include casings, coatings or other encapsulations which are to be swallowed at the same time or which may be expected to be swallowed. The expression “orally consumable product” covers ready-to-eat foodstuffs and feeds, that is to say foodstuffs or feeds that are already complete in terms of the substances that are important for the taste. The expressions “ready-to-eat foodstuff” and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds.

Preferred food compositions according to the invention also include “semi-finished products”. Within the context of the present text, a semi-finished product is to be understood as being an orally consumable product which, because of a very high content of flavourings and taste-imparting substances, is unsuitable for use as a ready-to-eat orally consumable product (in particular foodstuff or feed). Only by mixing with at least one further constituent (e.g. by reducing the concentration of the flavourings and taste-imparting substances in question) and optionally further process steps (e.g. heating, freezing) is the semi-finished product converted into a ready-to-eat orally consumable product (in particular foodstuff or feed). Examples of semi-finished products which may be mentioned here are

Food composition according to the invention preferably comprises one or more preparations for nutrition or enjoyment purposes. These include in particular (reduced-calorie) baked goods (e.g. bread, dry biscuits, cakes, other baked articles), confectionery (e.g. chocolates, chocolate bars, other products in bar form, fruit gums, dragées, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks or other foods. The preparations within the scope of the invention can also be used in the form of semi-finished products for the production of further preparations for nutrition or enjoyment purposes. The preparations within the scope of the invention can also be in the form of capsules, tablets (uncoated and coated tablets, e.g. enteric coatings), dragées, granules, pellets, solids mixtures, dispersions in liquid phases, in the form of emulsions, in the form of powders, in the form of solutions, in the form of pastes, or in the form of other preparations which can be swallowed or chewed, and in the form of food supplements.

The preparations can also be in the form of capsules, tablets (uncoated and coated tablets, e.g. enteric coatings), dragées, granules, pellets, solids mixtures, dispersions in liquid phases, in the form of emulsions, in the form of powders, in the form of solutions, in the form of pastes, or in the form of other preparations which can be swallowed or chewed, for example in the form of food supplements.

The semi-finished products are generally used for the production of ready-to-use or ready-to-eat preparations for nutrition or enjoyment purposes.

Further constituents of a ready-to-eat preparation or semi-finished product for nutrition or enjoyment purposes can be conventional base substances, auxiliary substances and additives for foods or enjoyment foods, for example water, mixtures of fresh or processed, vegetable or animal base or raw substances (e.g. raw, roast, dried, fermented, smoked and/or boiled meat, bone, cartilage, fish, vegetables, herbs, nuts, vegetable juices, vegetable pastes or mixtures thereof), digestible or non-digestible carbohydrates (e.g. sucrose, maltose, fructose, glucose, dextrins, amylose, amylopectin, inulin, xylans, cellulose, tagatose), sugar alcohols (e.g. sorbitol, erythritol), natural or hardened fats (e.g. tallow, lard, palm fat, cocoa fat, hardened vegetable fat), oils (e.g. sunflower oil, groundnut oil, maize germ oil, olive oil, fish oil, soya oil, sesame oil), fatty acids or their salts (e.g. potassium stearate), proteinogenic or non-proteinogenic amino acids and related compounds (e.g. γ-aminobutyric acid, taurine), peptides (e.g. glutathione), natural or processed proteins (e.g. gelatin), enzymes (e.g. peptidases), nucleic acids, nucleotides, taste correctors for unpleasant taste impressions, further taste modulators for further, generally not unpleasant taste impressions, other taste-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacylglycerols, gum arabic), stabilisers (e.g. carrageenan, alginate), preservatives (e.g. benzoic acid and its salts, sorbic acid and its salts), antioxidants (e.g. tocopherol, ascorbic acid), chelators (e.g. citric acid), organic or inorganic acidifying agents (e.g. acetic acid, phosphoric acid), additional bitter substances (e.g. quinine, caffeine, limonene, amarogentine, humulone, lupulone, catechols, tannins), substances that prevent enzymatic browning (e.g. sulfite, ascorbic acid), ethereal oils, plant extracts, natural or synthetic colourings or colouring pigments (e.g. carotinoids, flavonoids, anthocyans, chlorophyll and derivatives thereof), spices, trigeminally active substances or plant extracts containing such trigeminally active substances, synthetic, natural or nature-identical flavourings or odorants as well as odour correctors.

Food compositions according to the invention, for example those in the form of preparations or semi-finished products, preferably comprise a flavour composition in order to complete and refine the taste and/or odour. A preparation can comprise as constituents a solid carrier and a flavour composition. Suitable flavour compositions comprise, for example, synthetic, natural or nature-identical flavourings, odorants and taste-imparting substances, reaction flavourings, smoke flavourings or other flavour-giving preparations (e.g. protein (partial) hydrolysates, preferably protein (partial) hydrolysates having a high arginine content, barbecue flavourings, plant extracts, spices, spice preparations, vegetables and/or vegetable preparations) as well as suitable auxiliary substances and carriers. Particularly suitable here are the flavour compositions or constituents thereof which produce a roasted, meaty (in particular chicken, fish, seafood, beef, pork, lamb, mutton, goat), vegetable-like (in particular tomato, onion, garlic, celery, leek, mushroom, aubergine, seaweed), spicy (in particular black and white pepper, cardamom, nutmeg, pimento, mustard and mustard products), fried, yeast-like, boiled, fatty, salty and/or pungent flavour impression and accordingly can enhance the spicy impression. The flavour compositions generally comprise more than one of the mentioned ingredients.

The food compositions of the present invention are preferably selected from the group comprising
confectionery, preferably reduced-calorie or calorie-free confectionery, preferably selected from the group comprising muesli bar products, fruit gums, dragées, hard caramels and chewing gum,
non-alcoholic drinks, preferably selected from the group comprising green tea, black tea, (green, black) tea drinks enriched with (green, black) tea extracts, rooibos tea, other herbal teas, fruit-containing low-sugar or sugar-free soft drinks, isotonic drinks, nectars, fruit and vegetable juices, fruit and vegetable juice preparations,
instant drinks, preferably selected from the group comprising instant (green, black, rooibos, herbal) tea drinks,
cereal products, preferably selected from the group comprising low-sugar and sugar-free breakfast cereals and muesli bars,
dairy products, preferably selected from the group comprising reduced-fat and fat-free milk drinks, yoghurt, kefir, whey, buttermilk and ice-cream,
products made from soy protein or other soybean fractions, preferably selected from the group comprising soy milk, products produced from soy milk, drinks containing isolated or enzymatically treated soy protein, drinks containing soy flour, preparations containing soy lecithin, products produced from preparations containing soy lecithin and mixtures with fruit preparations and optionally flavours,
sweetener preparations, tablets and sachets,
sugar-free dragées,
ice-cream, with or without milk-based constituents, preferably sugar-free.

### Sweeteners

The term “sweeteners” here denotes substances having a relative sweetening power of at least 25, based on the sweetening power of sucrose (which accordingly has a sweetening power of 1). Sweeteners to be used in an orally consumable product (in particular foodstuff, feed or medicament) according to the invention (a) are preferably non-cariogenic and/or have an energy content of not more than 5 kcal per gram of the orally consumable product.

Advantageous sweeteners in a preferred orally consumable product (in particular foodstuff, feed or medicament) according to the invention are selected from the following groups:
(a) Naturally occurring sweeteners, preferably selected from the group comprising miraculin, monellin, mabinlin, thaumatin, curculin, brazzein, pentaidin, D-phenylalanine, D-tryptophan, and extracts or fractions obtained from natural sources, comprising those amino acids and/or proteins, and the physiologically acceptable salts of those amino acids and/or proteins, in particular the sodium, potassium, calcium or ammonium salts, neohesperidin dihydrochalcone, naringin dihydrochalcone, stevioside, steviolbioside, rebaudiosides, in particular rebaudioside A, rebaudioside B, rebaudioside C, rebaudioside D, rebaudioside E, rebaudioside F, rebaudioside G, rebaudioside H, dulcosides and rubusoside, suavioside A, suavioside B, suavioside G, suavioside H, suavioside I, suavioside J, baiyunoside 1, baiyunoside 2, phlomisoside 1, phlomisoside 2, phlomisoside 3 and phlomisoside 4, abrusoside A, abrusoside B, abrusoside C, abrusoside D, cyclocaryoside A and cyclocaryoside I, osladin, polypodoside A, strogin 1, strogin 2, strogin 4, selligueain A, dihydroquercetin 3-acetate, perillartin, telosmoside A₁₅, periandrin I-V, pterocaryosides, cyclocaryosides, mukuroziocides, trans-anethole, trans-cinnamaldehyde, bryosides, bryonosides, bryonodulcosides, carnosiflosides, scandenosides, gypenosides, trilobatin, phloridzin, dihydroflavanols, hematoxylin, cyanin, chlorogenic acid, albiziasaponin, telosmosides, gaudichaudioside, mogrosides, mogroside V, hernandulcins, monatin, phyllodulcin, glycyrrhetinic acid and derivatives thereof, in particular glycosides thereof such as glycyrrhizine, and the physiologically acceptable salts of those compounds, in particular the sodium, potassium, calcium or ammonium salts; and
   extracts or concentrated fractions of the extracts, selected from the group comprising thaumatococcus extracts (katamfe plant), extracts from Stevia ssp. (in particular Stevia rebaudiana), swingle extracts (Momordica or Siratia grosvenorii, Luo-Han-Guo), extracts from Glycyrrhiza ssp. (in particular Glycyrrhiza glabra), extracts from Rubus ssp. (in particular Rubus suavissimus), citrus extracts and extracts from Lippia dulcis;
(b) Synthetic sweet-tasting substances, preferably selected from the group comprising magap, sodium cyclamate or other physiologically acceptable salts of cyclamic acid, acesulfame K or other physiologically acceptable salts of acesulfame, neohesperidin dihydrochalcone, naringin dihydrochalcone, saccharin, saccharin sodium salt, aspartame, superaspartame, neotame, alitame, advantame, perillartin, sucralose, lugduname, carrelame, sucrononate and sucrooctate.

### Thickeners

Advantageous thickeners in a preferred orally consumable product (in particular foodstuff, feed or medicament) according to the invention are selected from the group comprising: crosslinked polyacrylic acids and derivatives thereof, polysaccharides and derivatives thereof, such as xanthan gum, agar-agar, alginates or tyloses, cellulose derivatives, for example carboxymethylcellulose or hydroxycarboxymethylcellulose, fatty alcohols, monoglycerides and fatty acids, polyvinyl alcohol and polyvinylpyrrolidone.

Preference is given according to the invention to an orally consumable product (in particular foodstuff or feed) which comprises milk thickened with lactic acid bacteria and/or cream thickened with lactic acid bacteria and which preferably is selected from the group comprising yoghurt, kefir and quark.

A food composition according to the invention comprising milk thickened with lactic acid bacteria and/or cream thickened with lactic acid bacteria is advantageously an orally consumable product which comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HN019, Lactobacillus acidophilus LA5, Lactobacillus acidophilus NCFM, Lactobacillus johnsonii Lal, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

### Additives for Chewing Gums

Particular preference is given to an orally consumable product (in particular foodstuff, feed or medicament) according to the invention that is a chewing gum and comprises a chewing-gum base. The chewing-gum base is preferably selected from the group comprising chewing-gum or bubble-gum bases. The latter are softer, so that gum bubbles can also be formed therewith. Preferred chewing-gum bases according to the invention include, in addition to the natural resins or the natural latex chicle that are traditionally used, elastomers such as polyvinyl acetate (PVA), polyethylene, (low or medium molecular weight) polyisobutene (PIB), polybutadiene, isobutene-isoprene copolymers (butyl rubber), polyvinyethyl ether (PVE), polyvinylbutyl ether, copolymers of vinyl esters and vinyl ethers, styrene-butadiene copolymers (styrene-butadiene rubber, SBR) or vinyl elastomers, for example based on vinyl acetate/vinyl laurate, vinyl acetate/vinyl stearate or ethylene/vinyl acetate, as well as mixtures of the mentioned elastomers, as described, for example, in EP 0 242 325, U.S. Pat. Nos. 4,518,615, 5,093,136, 5,266,336, 5,601,858 or U.S. Pat. No. 6,986,709. In addition, chewing-gum bases that are preferably to be used according to the invention preferably comprise further constituents such as, for example, (mineral) fillers, plasticisers, emulsifiers, antioxidants, waxes, fats or fatty oils, such as, for example, hardened (hydrogenated) vegetable or animal fats, mono-, di- or tri-glycerides. Suitable (mineral) fillers are, for example, calcium carbonate, titanium dioxide, silicon dioxide, talcum, aluminium oxide, dicalcium phosphate, tricalcium phosphate, magnesium hydroxide and mixtures thereof. Suitable plasticisers, or agents for preventing adhesion (detackifiers), are, for example, lanolin, stearic acid, sodium stearate, ethyl acetate, diacetin (glycerol diacetate), triacetin (glycerol triacetate), triethyl citrate. Suitable waxes are, for example, paraffin waxes, candelilla wax, carnauba wax, microcrystalline waxes and polyethylene waxes. Suitable emulsifiers are, for example, phosphatides such as lecithin, mono- and di-glycerides of fatty acids, for example glycerol monostearate.

Chewing gums according to the invention (in particular as disclosed above) preferably comprise constituents such as sugars of different types, sugar substitutes, other sweet-tasting substances, sugar alcohols (in particular sorbitol, xylitol, mannitol), ingredients having a cooling effect, taste correctors for unpleasant taste impressions, further taste-modulating substances (e.g. inositol phosphate, nucleotides such as guanosine monophosphate, adenosine monophosphate or other substances such as sodium glutamate or 2-phenoxypropionic acid), humectants, thickeners, emulsifiers, stabilisers, odour correctors and flavours (e.g. eucalyptus-menthol, cherry, strawberry, grapefruit, vanilla, banana, citrus, peach, blackcurrant, tropical fruits, ginger, coffee, cinnamon, combinations (of the mentioned flavours) with mint flavours as well as spearmint and peppermint on their own). The combination inter alia of the flavours with further substances that have cooling, warming and/or mouth-watering properties is of particular interest.

### INDUSTRIAL APPLICATION

Another object of the present invention refers to a method for
improving antimicrobial stability of a cosmetic or foodstuff preparation; and/or
masking unpleasant flavors, particularly unpleasant flavors of fluorine derivatives and bitter compounds; and/or
enhancing and/or boosting the performance of a flavor, a sweetener or a soothing agent encompassing the step of adding a working amount of the composition according to the present invention to said preparation. Typically the compositions are added in amounts of from 0.01 to 2 wt.-percent and preferably from about 0.1 to 1 wt.-percent—calculated on the preparations.

Also claimed is the use of the compositions according to the present invention for
improving the antimicrobial stability of a cosmetic or foodstuff preparation; and/or
masking unpleasant flavors, particularly unpleasant flavors of fluorine derivatives and bitter compounds; and/or
enhancing and/or boosting the performance of a flavor, a sweetener or a soothing agent.

For the sake of good order it is emphasized that all preferred embodiments, namely mixtures, compositions, ratio and the like also apply to the method and the use as claimed above. Therefore, any repetition is superfluous.

### Modulation of Inflammatory Response by Substituted Azacycles

The human immune response is a highly orchestrated and fine-tuned system reacting to any non-host particle. It detects and battles hostile microorganisms or toxigenic substances by modulating the human host physiological functions as well as stimulating innate and acquired immune responses for the clearance of the foreign particles. Initial host immune events include sometimes unpleasant reactions such as itching, swelling, and reddening of affected areas and there is high interest in the cosmetic industry to detect multi-functional mixtures with additional soothing activity besides their primary property counteracting aforementioned reactions. In this regard, cytokines like IL-1 beta, IL-6, TNF alpha and PGE2 are known to mediate vasodilation, fever, pain and reddening, the classical physiological signals for inflammation. Macrophage cells are key for first line human defences. They orchestrate the response by means of signalling molecules and are a well-known model system to investigate soothing activities of anti-inflammatory agents.

### Mouse Macrophage Cell Culture

RAW 264.7 (mouse macrophage tumor cells) cells were provided from the University of Cordoba in Spain and maintained in RPMI1640 medium containing FCS.

### Cell Stimulation

Cells are seeded in 24-well-plates (approx. 500 000 cells/ml in 1 ml) for ELISA experiments.

Cells are incubated without or with LPS (10 ng/ml) for 24 h. The pyrozol derivates or additives such as Thymol or Menthol (3 doses, 1, 10, 100 μM) are added 30 min before LPS treatment as single dose or in combination. After 24 h, supernatants are removed, centrifuged, and stored at −80° C. After 24 h, supernatants are removed, centrifuged, and investigated for PGE2 concentrations in EIA (PGE2, from Cayman, isoprostane from Cayman) or ELISA (IL-1B, IL-6, TNF-alpha, Biotechne)). Each dose is investigated 2 times.

Optimal dose range for additives and substituted azacycles was determined in a preliminary assays dose finding assay (data not shown). Thus, test items are investigated at 10 μM 100 μM or any combination of the two doses for additive effects. Synergism is defined as additive effect surpassing the hypothetical mathematical value of effective doses. The hypothetical value is depicted in all graphs as dashed line.

Azacycles used in the experiments are referenced as followed:
CoNo11: 2-methylsulfanyl-1-[(1R)-2-[5-(p-tolyl)-1H-imidazol-2-yl]-1-piperidyl]-propan-1-one
CoNo 164: 2-methyl-1-[2-[5-(p-tolyl)-1H-imidazol-2-yl]-1-piperidyl]butan-1-one
CoNo 166: (2S)-2-methyl-1-[2-[5-(p-tolyl)-1H-imidazol-2-yl]-1-piperidyl]butan-1-one Cytotoxicity

Cells are seeded in 96 well plates (approx. 5000 cells/well in 200 μl) for cytotoxicity testing. Cells are incubated with LPS and the test items (3 doses, n=2). After 24 h, cytotoxicity is evaluated in the wells using the AlamarBlue methods.

### SHORT DESCRIPTION OF THE FIGURES

FIG. 1 depicts impact of substance CoNo11 either at single concentration of 10 μM or 100 μM or in combination with Thymol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects.

FIG. 2 depicts impact of substance CoNo11 either at single concentration of 10 μM or 100 μM or in combination with Menthol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects

FIG. 3 depicts impact of substance CoNo166 either at single concentration of 10 μM or 100 μM or in combination with Thymol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects.

FIG. 4 depicts impact of substance CoNo166 either at single concentration of 10 μM or 100 μM or in combination with Menthol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects.

FIG. 5 depicts impact of substance CoNo166 either at single concentration of 10 μM or 100 μM or in combination with Thymol at 10 μM or 100 μM on the LPS-stimulated, pro-inflammatory synthesis of PGE2 in RAW macrophages. Graph shows outcome of enzyme-linked assay done in duplicates referenced to the LPS-stimulated control as 100%. Dashed line shows hypothetical value for additive effects.

FIG. 6 depicts impact of substance CoNo164 either at single concentration of 10 μM or 100 μM or in combination with Thymol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects.

FIG. 7 depicts impact of substance CoNo164 either at single concentration of 10 μM or 100 μM or in combination with Menthol at 10 μM or 100 μM on the LPS-stimulated, pro-inflammatory synthesis of IL-1B in RAW macrophages. Graph shows outcome of enzyme-linked assay done in duplicates referenced to the LPS-stimulated control as 100%. Dashed line shows hypothetical value for additive effects.

FIG. 8 depicts impact of substance CoNo164 either at single concentration of 10 μM or 100 μM or in combination with Thymol at 10 μM or 100 μM on the LPS-stimulated, pro-inflammatory synthesis of PGE2 in RAW macrophages. Graph shows outcome of enzyme-linked assay done in duplicates referenced to the LPS-stimulated control as 100%. Dashed line shows hypothetical value for additive effects.

FIG. 9 depicts impact of substance CoNo11 either at single concentration of 10 μM or 100 μM or in combination with Thymol at 10 μM or 100 μM on the LPS-stimulated, pro-inflammatory synthesis of all tested pro-inflammatory marker tested in in RAW macrophages. Graph shows outcome of enzyme-linked assay done in duplicates referenced to the LPS-stimulated control as 100%. Dashed line shows hypothetical value for additive effects.

### CONCLUSION

The skilled person is aware that synergistic effects exceed the hypothetic additive effect of single substances when used in combination.

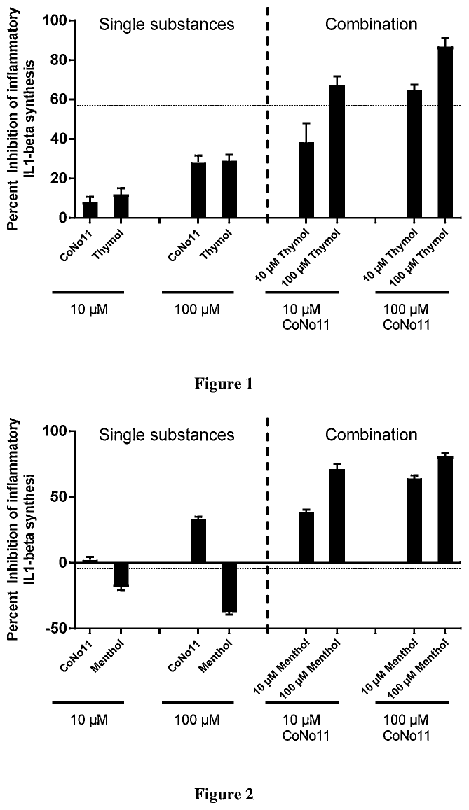

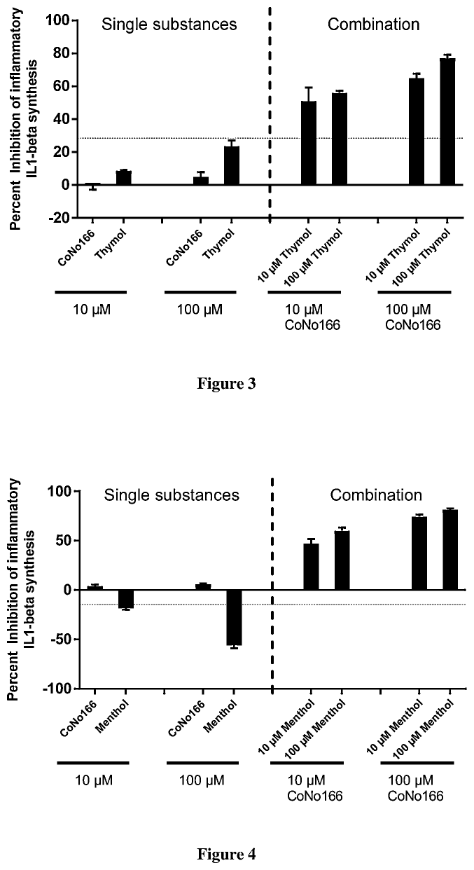

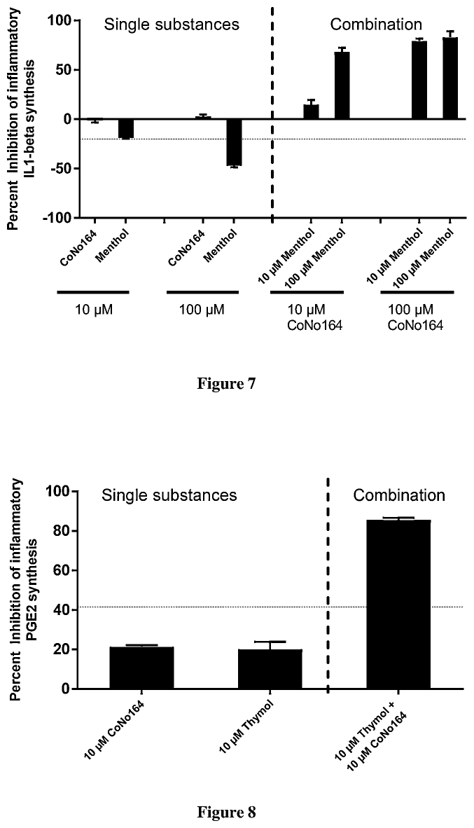

Some of the most active TRMP8 agonists the azacycles CoNo11, CoNo166, CoNo164 when used in combination with Thymol a strong synergistic soothing effect on LPS-stimulated IL-1 beta synthesis is depicted in FIG. 1, FIG. 3, FIG. 6, and FIG. 8. Application of Menthol resulted in an even more distinct synergistic effect. Menthol applied as single substance stimulates IL-1 beta synthesis as visualized by negative values while in combination with CoNo11, CoNo166 and CoNo164 (FIG. 2, FIG. 4 and FIG. 7) results in strong inhibition of IL-1 beta synthesis in all tested combinations.

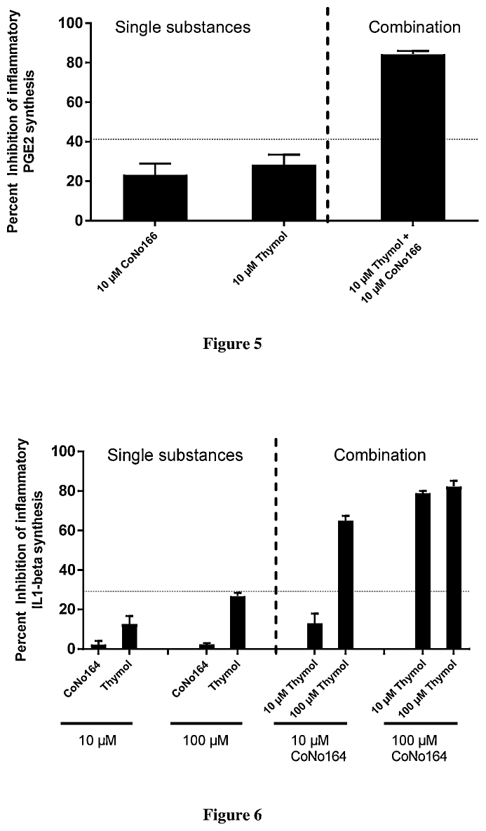

Moreover, for some substances we have also an anti-inflammatory effect on PGE2. For sake of simplicity only relevant data is depicted. Substituted azacycles CoNo166 and CoNo164 at a concentration of 10 μM in combination with 10 μM Thymol synergistically mitigated PGE2 synthesis (FIG. 5 and FIG. 8).

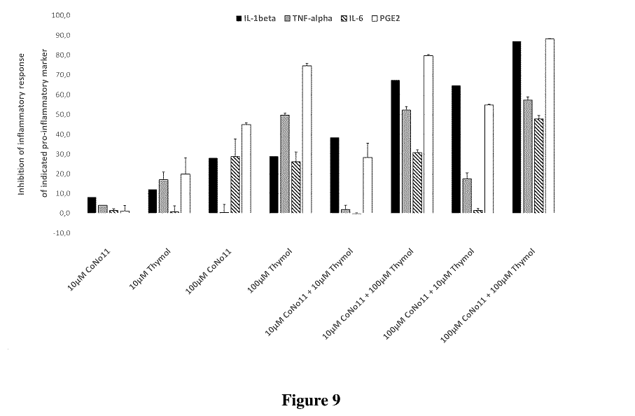

All depicted effects are due to modulatory capacity of the indicated molecules and not caused by cytotoxic effects (data not shown). Furthermore, IL-1 beta and PGE2 mitigation of synthesis is substance-specific as neither TNF alpha or IL-6 are affected in a similar way (example FIG. 9).

### EXAMPLES

### Formulation Examples for Cosmetic Compositions

The following examples F1 to F55 show various formulations for cosmetic compositions. “Composition 1” stands for any mixture according to Examples 1 to 38 as defined above.

TABLE F1
Liquid soap; transparent (Amounts in % b.w.)

| Ingredients | INCI Name | Amount |
|---|---|---|
| Tagat O 2 | PEG-20 Glyceryl Oleate | 2.5 |
| Coconut oil diethanolamine | Cocamide DEA | 5.0 |
| condensate | | |
| Abil B 8842 | Cyclomethicone | 0.5 |
| Sodium laurylethersulfate. | Sodium Laureth Sulfate | 35.0 |
| 28% | | |
| Tego-Betaine L7 | Cocamidopropyl Betaine | 5.0 |
| Soap. 25% | Coconut acid. Potassium | 20.0 |
| | salt. Potassium | |
| | Oleate | |
| Hydrolite® 5 | 1,2-propanediol | 0.4 |
| Composition 1 | | 0.3 |
| Water | Water | Ad 100 |

TABLE F2
Syndet soap; liquid (Amounts in % b.w.)

| Ingredients | INCI Name | Amount |
|---|---|---|
| Elfan OS 46 | Sodium Olefin C14-C16 Sulfonate | 35.5 |
| Armoteric LB | Lauryl Betaine | 8.0 |
| Elfan SG | | 10.0 |
| Elfacos GT 282 L | Talloweth-60 Myristyl Glycol | 3.0 |
| PCL-Liquid 100 | Cetearyl Ethylhexanoate | 4.0 |
| Hydrolite ® 5 Green | Hydrolite ® 5 Green | 0.4 |
| Preservative | Methylchloroisothiazolinone | 0.1 |
| Composition 1 | | 0.4 |
| Water | Water | Ad 100 |

TABLE F3
Cosmetic lotion for body wash (Amounts in % b.w.)

| Ingredients | INCI Name | Amount |
|---|---|---|
| Lumerol K 28 | Disodium Laureth Sulfosuccinate. | 33.0 |
| | Cocamidopropyl Betaine. | |
| | Magnesium Lauryl Sulfate | |
| Amphotensid B 4 | Cocamidopropyl Betaine | 10.0 |
| Perlglanzmittel | MIPA-Pareth-25 Sulfate. | 4.0 |
| GM 4055 | Glycol Stearate | |
| Sodium Chloride | Sodium Chloride | 2.0 |
| Avocado oil | Persea Gratissima | 3.0 |
| | (Avocado) Oil | |
| Water | Water | Ad 100 |
| Hydrolite ® 5 | Hydrolite ® 5 Green | 0.5 |
| Euxyl ® K727 | Phenoxyethanol. Methyldibromo | 0.3 |
| | Glutaronitrile Isothiazolinones | |
| Composition 1 | | 0.6 |

TABLE F4
Cosmetic lotion for body wash with Triclosan
(Amounts in % b.w.)

| Ingredients | INCI Name | Amount |
|---|---|---|
| Texapon N 25 | Sodium Laureth Sulfate | 37.5 |
| Lamepon S | Potassium Cocoyl Hydrolyzed | 28.0 |
| | Collagen | |
| Lamesoft LMG | Hydrogenated Tallow Glycerides. | 5.0 |
| | TEA-Co-coyl Hydrolyzed Collagen | |
| Lamesoft 156 | Glyceryl Laurate. TEA-Cocoyl | 5.0 |
| | Hydrolyzed Collagen | |
| Sodium Chloride | Sodium Chloride | 1.7 |
| Irgasan DP 300 | Triclosan | 0.5 |
| Water | Water | Ad 100 |
| Hydrolite ® 5 | Hydrolite ® 5 Green | 0.3 |
| Euxyl ® K703 | Phenoxyethanol. Benzoic Acid. | 0.4 |
| | Dehydroacetic Acid | |
| Composition 1 | | 0.4 |

TABLE F5
Intimate wash (Amounts in % b.w.)

| Ingredients | INCI Name | Amount |
|---|---|---|
| Tegobetaine HS | Cocamidopropyl Betaine. | 15.0 |
| | Glyceryl Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid. 80% | Lactic Acid | 0.1 |
| Water | Water | Ad 100 |
| Hydrolite ® 5 | Hydrolite ® 5 Green | 0.2 |
| Euxyl ® K700 | Potassium Sorbate. | 0.3 |
| | Benzyl Alcohol. | |
| | Phenoxyethanol | |
| Composition 1 | | 0.5 |

TABLE F6
Liquid soap (Amounts in % b.w.)

| Ingredient | INCI | Amount |
|---|---|---|
| Deionized water | Water | 2.0 |
| Soap bases mix | Sodium tallowates/palmitates | 95.8 |
| Titanium dioxide | Titanium dioxide | 1.0 |
| Hydrolite ® 5 | Hydrolite ® 5 Green | 1.2 |
| Preservatives | Phenoxyethanol | 0.5 |
| Composition 1 | | 0.5 |

TABLE F7
Shampoo (Amounts in % b.w.)

| Ingredients | Amount |
|---|---|
| Sodium lauryl ether sulfate | 12 |
| (e.g. Texapon NSO) | |
| Cocamidopropyl betaine | 2 |
| (e.g. Dehyton K) | |
| Sodium chloride | 1.4 |
| Citric acid | 1.3 |
| 1,2-pentanediol (Hydrolite ® 5) | 0.3 |
| Phenoxyethanol. methyl -. ethyl -. | 0.5 |
| butyl- and propylparaben | |
| Composition 1 | 0.5 |
| Water | Ad 100 |

TABLE F8
2-in-1 Shampoo (Amounts in % b.w.)

| Ingredients | INCI Name | Amount |
|---|---|---|
| Deionized water | Water | Ad 100 |
| Plantacare PS 10 | Sodium Laureth Sulfate. | 20.0 |
| | Lauryl Glucoside | |
| Euperlan PK 771 | Glycol Distearate. | 6.0 |
| | Sodium Lauryl Sulfate. | |
| | Cocamide MEA. Laureth-10 | |
| Sodium chloride | Sodium Chloride | 1.4 |
| Citric acid monohydrate | Citric acid | 0.1 |
| crystalline | | |
| Hydrolite ® 5 | Hydrolite ® 5 Green | 0.5 |
| Dragocid Liquid | Phenoxyethanol, Parabens | 0.5 |
| Composition 1 | | 0.5 |

TABLE F9
Anti-dandruff Shampoo (Amounts in % b.w.)

| Ingredients | Amount |
|---|---|
| Climbazole | 0.50 |
| Sodium Laureth Sulfate | 37.00 |
| Cocamidopropyl Betaine | 8.00 |
| PEG-6 Caprylic/Capric Glycerides | 2.50 |
| Laureth-2 | 2.00 |
| Water (Aqua). Glycerol. Thymus Vulgaris | 0.50 |
| (Thyme). Flower/Leaf Extract | |
| Rosmarinus Officinalis (Rosemary) | 0.50 |
| Leaf Water. Water (Aqua). Butylene | |
| Glycol. Pentylene Glycol | |
| Bisabolol | 0.10 |
| Panthenol | 0.50 |
| Polyquaternium-10 | 0.40 |
| 1,2-pentanediol (Hydrolite ® 5) | 0.50 |
| Phenoxyethanol. Methylparaben. Ethylparaben. | |
| Butylparaben. Propylparaben. Isobutylparaben | 0.70 |
| Composition 1 | 0.30 |
| Water (Aqua) | Ad 100 |

TABLE F10
Hair conditioner with Crinipan. rinse-off (Amounts in % b.w.)

| Ingredients | INCI Name | Amount |
|---|---|---|
| Lanette ® O | Cetearyl Alcohol | 4.00 |
| Dragoxat 89 | Ethylhexyl Isononanoate | 2.00 |
| Genamin ® KDM-P | Behentrimonium Chloride | 1.00 |
| SF 1550 | Phenyl Trimethicone | 0.10 |
| Neo Heliopan ® BB | Benzophenone-3 | 0.10 |
| Crinipan ® AD | Climbazole | 0.80 |
| Glycerol 99.5 P. | Glycerol | 6.00 |
| Water | Water (Aqua) | Ad 100 |
| Actipone ® | Water (Aqua). Butylene | 0.50 |
| Alpha Pulp | Glycol. Malic Acid. Actinidia | |
| | Chinensis (Kiwi)Fruit Juice. | |
| | Citrus. Aurantium Dulcis (Orange). | |
| | Juice. Citrus Paradisi (Grapefruit) | |
| | Juice. Pyrus Malus (Apple) Juice. | |
| | Trideceth-9. PrunusAmygdalus | |
| | Dulcis (Sweet Almond) Seed Extract | |
| Extrapone ® | Propylene Glycol. Water (Aqua). | 0.50 |
| Bamboo P | Butylene Glycol. Bambusa | |
| | Vulgaris Shoot Extract | |
| Sodium Hydroxide | Sodium Hydroxide | 0.40 |
| 10% solution | | |
| Colour I | Colour | 0.60 |
| Colour II | Colour | 0.30 |
| Hydrolite ® 5 | Hydrolite ® 5 Green | 0.40 |
| Preservative | Methylparaben | 0.20 |
| Composition 1 | Hydroxyacetophenone | 0.70 |

TABLE F11
Sprayable hair conditioner with zinc pyrithrione.
leave-on (Amounts in % b.w.)

| Ingredients | INCI Name | Amount |
|---|---|---|
| Monomuls 60-35 C | Hydrogenated Palm Glycerides | 1.70 |
| Cetiol OE | Dicaprylyl Ether | 7.20 |
| Abil 100 | Dimethicone | 3.60 |
| Dehyquart F 75 | Distearoylethyl Hydroxyethylmonium. | 4.00 |
| | Methosulfate. Cetearyl Alcohol | |
| Eumulgin B1 | Ceteareth-12 | 3.50 |
| Cetiol S | Diethylhexylcyclohexane | 7.20 |
| D-Panthenol | Panthenol | 0.10 |
| Glycerol 99.5 P. | Glycerol | 1.50 |
| Water | Water (Aqua) | Ad 100 |
| Actipone ® Rosemary | Water (Aqua). Propylene. | 0.10 |
| | Glycol. Rosmarinus Officinalis. | |
| | (Rosemary) Leaf Extract | |
| Frescolat ® ML Cryst. | Menthyl Lactate | 0.50 |
| Dragosantol100 | Bisabolol | 0.10 |
| Zinc Omadine | Zinc pyrithione | 0.10 |
| Hydrolite ® 5 | Hydrolite ® 5 | 0.40 |
| Phenonip ® | phenoxyethanol. methylparaben. | 0.30 |
| | ethylparaben. butylparaben. | |
| | propylparaben. isobutylparaben | |
| Composition 1 | | 0.5 |

TABLE F12
Hair conditioner with UV protection (Amounts in % b.w.)

| Ingredients | I NCI Name | Amount |
|---|---|---|
| Renex PEG 6000 | PEG-150 | 2.50 |
| Hair Conditioner Base | Cetyl alcohol. behentrimonium | 3.00 |
| | chloride. Triticum Vulgare | |
| | (Wheat) bran extract. linoleic acid | |
| PCL-Solid | Stearyl heptanoate. stearyl caprylate | 0.50 |
| Dow Corning 5200 | Laurylmethicone copolyol | 0.50 |
| Natrosol 250 HR | Hydroxyethylcellulose | 0.50 |
| Benzophenone-4 | Benzophenone-4 | 1.00 |
| Neo Heliopan AP | Disodiumphenyldibenz-imidazole | 1.00 |
| | tetrasulphonate | |
| Amino methyl propanol | Amino methyl propanol | 2.00 |
| Dow Corning 949 cationic | Amodimethicone. cetrimonium | 2.00 |
| emulsion | chloride. trideceth-12 | |
| Hydrolite ® 5 | Hydrolite ® 5 | 0.80 |
| 1.2-hexanediol | 1.2-hexanediol | 0.50 |
| Composition 1 | | 0.50 |
| Water | Water (Aqua) | Ad 100 |

TABLE 13
Shower gel (Amounts in % b.w.)

| Ingredients | INCI | Amount |
|---|---|---|
| Deionized water | Water | Ad 100 |
| Plantacare PS 10 | Sodium Laureth Sulfate. | 20.0 |
| | Lauryl Glucoside | |
| Sodium chloride | Sodium Chloride | 1.4 |
| Citric acid monohydrate | Citric Acid | 1.3 |
| crystalline | | |
| Hydrolite ® 5 | Hydrolite ® 5 | 0.6 |
| SymDiol ®68 | 1.2-hexanediol. | 0.4 |
| Composition 1 | Caprylyl glycol | 0.4 |

TABLE F14
Shaving foam (Amounts in % b.w.)

| Ingredients | Amount |
|---|---|
| Dem. Water | 77.2 |
| Triethanolamine | 4.0 |
| Edenor L2 SM (Stearinic acid. | 5.3 |
| Palmitinic acid) (Cognis) | |
| Laureth-23 | 3.0 |
| Stearylalcohol | 0.5 |
| Sodium lauryl sulfate | 3.0 |
| Extrapone Seaweed | 1.0 |
| (water. propylene glycol. | |
| potassium iodide. Fucus | |
| Vesiculosus Extract) | |
| Dragosantol (Bisabolol. Farnesol) | 0.1 |
| 1,2-pentanediol (Hydrolite ® 5) | 1.0 |
| Euxyl ® K220 (Methylisothiazolinone. | 0.6 |
| Ethylhexylglyerol) | |
| Composition 1 | 0.3 |
| Propane. butane 4.2 Bar | 4.0 |

TABLE F15
Depilatory cream (Amounts in % b.w.)

| Ingredients | Amount |
|---|---|
| Cetearyl alcohol | 10.0 |
| Ceteareth-12 | 2.0 |
| PCL-Liquid (Cetearylethylhexanoate. | 3.0 |
| Isopropylmyristate) | |
| Dragosantol (Bisabolol. Farnesol) | 0.1 |
| Edenor L2 SM (Stearinic acid. | 1.0 |
| Palmitinic acid) | |
| Dem. Water | 52.2 |
| Urea | 5.0 |
| Dem. Water | 10.0 |
| Calcium thioglycolate | 6.0 |
| Sodium hydroxide solution. 10% | 10.0 |
| 1,2-pentanediol (Hydrolite ® 5) | 0.5 |
| Neo Dragocid Powder (Methyl parabene. | 0.2 |
| sorbinic acid. Dehydro acetic acid. | |
| Propyl parabene) | |
| Composition 1 | 0.4 |

TABLE F16
After Shave Tonic (Amounts in % b.w.)

| Ingredients | INCI | Amount |
|---|---|---|
| SymSol ® PF-3 | Water (Aqua). Pentylene Glycol. | 3.00 |
| | Sodium Lauryl Sulfoacetate. | |
| | SodiumOleoyl Sarcosinate. | |
| | Sodium Chloride. Disodium | |
| | Sulfoacetate. SodiumOleate. | |
| | Sodium Sulfate | |
| SymSitive ® 1609 | Pentylene Glycol. | 1.00 |
| | 4-t-Butylcyclohexan ol | |
| Frescolat ® ML | Menthyl Lactate | 0.30 |
| Glycerol 99.5 P. | Glycerol | 5.00 |
| Water | Water (Aqua) | Ad 100 |
| Extrapone ® Glacier | Glycerol. Water (Aqua) | 1.00 |
| Water GW | | |
| SymCalmin ® | Butylene Glycol. Pentylene | 0.50 |
| | Glycol. Hydroxyphenyl | |
| | Propamidobenzoic Acid | |
| Dragosine ® | Carnosine | 0.10 |
| Hydrolite ® 5 | Pentylene Glycol | 5.00 |
| Ethanol 96 % | Alcohol Denat. | 5.00 |
| Colour Pigment | Colour Pigment | 0.05 |
| Hydrolite ® 5 | 1,2-pentanediol | 0.15 |
| Composition 1 | Hydroxyacetophenone | 1.00 |

TABLE F17
Deodorant formulation in the form
of a roll-on gel (Amounts in % b.w.)

| Ingredients | Amount |
|---|---|
| 1.3-butylene glycol | 2.00 |
| PEG-40-hydrogenated castor oil | 2.00 |
| Hydroxyethylcellulose | 0.50 |
| 1,2-pentanediol (Hydrolite ® 5) | 0.30 |
| 1.3-propanediol | 0.50 |
| 3-phenylpropanol | 0.40 |
| Ethylhexyl glycerin | 0.10 |
| Composition 1 | 0.50 |
| Water | ad 100.00 |

TABLE F18
Clear deo anti-perspirant roll-on (Amounts in % b.w.)

| Ingredients | INCI | Amount |
|---|---|---|
| Methocel E4M | Hydroxypropyl Methylcellulose | 0.50 |
| Premium | | |
| Water | Water (Aqua) | Ad 100 |
| Neo-PCL Water | Trideceth-9. PEG-5 Ethylhexanoate. | 1.00 |
| Soluble N | Water (Aqua) | |
| Solubilizer | PEG-40 Hydrogenated Castor Oil. | 3.00 |
| | Trideceth-9. Propylene Glycol. | |
| | Water (Aqua) | |
| Deolite | Dimethyl Phenylpropanol. | 0.50 |
| | Pentylene Glycol | |
| Locron LW | Aluminium Chlorohydrate | 25.00 |
| Aloe Vera Gel | Aloe Barbadensis Leaf Juice | 1.00 |
| Concentrate 10/1 | | |
| Propylene Glycol -1.2 | Propylene Glycol | 4.00 |
| 99 P GC | | |
| Ethanol 96% | Alcohol Denat. | 30.00 |
| Hydrolite ® 5 | 1,2pentanediol | 1.00 |
| Composition 1 | Hydroxyacetophenone | 0.25 |

TABLE F19
Deodorant stick (Amounts in % b.w.)

| Ingredients | Amount |
|---|---|
| Sodium stearate | 8.00 |
| PPG-3 Myristyl ether | 70.00 |
| 1.2-propylene glycol | 10.00 |
| 1.1-dimethyl-3-phenylpropanol | 0.20 |
| 2-butyloctanoic acid | 0.20 |
| 1,2-pentanediol (Hydrolite ® 5) | 0.60 |
| Heptoxy-1.2-propanediol | 0.20 |
| Phenoxyethanol | 0.30 |
| Composition 1 | 0.50 |
| Water | Ad 100 |

TABLE F20
Zirconium suspensoid antiperspirant stick (Amounts in % b.w.)

| Ingredients | INCI | Amount |
|---|---|---|
| PCL Liquid 100 | Cetearyl ethylhexanonate | to 100 |
| Silicone Fluid 345 | Cyclomethicone | 10.00 |
| CRODACOL C90 | Cetyl Alcohol | 8.00 |
| SYNCROWAX HGLC | C18-36 Triglyceride | 8.00 |
| CRODAMOL PTC | Pentaerythritol Tetracaprylate/Caprate | 5.00 |
| SYNCROWAX HRC | Tribehenin | 4.00 |
| VOLPO N5 | Oleth-5 | 1.00 |
| Titanium Dioxide | | 1.00 |
| Rezal 36GP | Aluminium Tetrachlorohydrex GLY | 20.00 |
| Dry Flo C | Aluminium Starch Octenyl Succinate | 22.50 |
| Hydrolite ® 5 | 1,2-pentanediol | 0.60 |
| Preservative | Phenoxyethanol | 0.40 |
| Hexoxy-1.2-propanediol | | 0.10 |
| Composition 1 | | 0.40 |

TABLE F21
Deodorant pump spray with SymClariol (Amounts in % b.w.)

| Ingredients | INCI | Amount |
|---|---|---|
| SymClariol ® | Decylene Glycol | 0.50 |
| Solubilizer | PEG-40 Hydrogenated Castor Oil. | 4.00 |
| | Trideceth-9. Propylene Glycol. Water (Aqua) | |
| Neo-PCL Water Soluble N | Trideceth-9. PEG-5 Ethylhexanoate. Aqua | 1.50 |
| SymRelief ® | Bisabolol. Zingiber Officinale | 0.10 |
| | (Ginger) Root Extract | |
| Water | Water (Aqua) | Ad 100 |
| 1.2 Propylene Glycol | Propylene Glycol | 6.00 |
| Hydrolite ® 5 | 1,2-pentanediol | 0.40 |
| SymDiol ® 68 | 1.2-Hexanediol. Caprylyl Glycol | 0.20 |
| Composition 1 | | 0.40 |

TABLE F22
Antiperspirant formulations (Amounts in % b.w.)

| Ingredients | Amounts |
|---|---|
| Reach AZP-908 SUF | 24.00 |
| Cyclomethicone (Pentamer) | Ad 100 |
| Polydecene (Silkflo 364 NF) | 17.50 |
| Neo Heliopan OS (ethylhexyl salicylate) | 2.50 |
| L-Menthyl lactate (Frescolate ML) | 0.25 |
| Polyethylene | 3.00 |
| Hydrogenated castor oil | 2.00 |
| Promyristyl PM-3 | 7.00 |
| PEG-8 Distearate | 3.00 |
| Silicon dioxide (Cab-O-Sil M-5) | 1.00 |
| Stearyl alcohol | 15.00 |
| Octyldodecanol | 0.10 |
| 1,2-pentanediol (Hydrolite ® 5) | 0.80 |
| 3-Phenylpropanol | 0.40 |
| Composition 1 | 0.60 |

TABLE F23
Deodorant spray with Triclosan (Amounts in % b.w.)

| Ingredients | Amount |
|---|---|
| PEG-40-hydrogenated castor oil | 3.00 |
| Ethylhexylglycerol (Octoxyglycerol) | 0.80 |
| Ethanol | 40.00 |
| Citrate buffer | 0.50 |
| Triclosan ® (5-chloro-2-(2.4- | 0.25 |
| dichlorophenoxy)phenol) | |
| 1,2-pentanediol (Hydrolite ® 5) | 0.75 |
| Phenoxyethanol | 0.40 |
| Composition 1 | 0.40 |
| Water | Ad 100 |

TABLE F24
O/W lotion (Amounts in % b.w.)

| Ingredients | Amount |
|---|---|
| Paraffin oil | 5.00 |
| Isopropyl palmitate | 5.00 |
| Cetyl alcohol | 2.00 |
| Beeswax | 2.00 |
| Ceteareth-20 | 2.00 |
| PEG-20-glyceryl stearate | 1.50 |
| Glycerol | 3.00 |
| 1,2-pentanediol (Hydrolite ® 5) | 0.30 |
| Methylparaben | 0.30 |
| Composition 1 | 0.60 |
| Water | ad 100.00 |

TABLE F25
Body lotion (Amounts in % b.w.)

| Ingredients | Amount |
|---|---|
| Cetearyl Alcohol | 2.00 |
| Ethylhexyl Isononanoate | 5.00 |
| Cetearyl Ethylhexanoate. | 3.00 |
| Isopropyl Myristate | |
| Glyceryl Oleate Citrate. | 4.00 |
| Caprylic/Capric Triglyceride | |
| Water (Aqua) | 79.50 |
| Carbomer | 0.30 |
| Sodium Benzoate | 0.100 |
| Propylene Glycol | 5.00 |
| Sodium Hydroxide 30% solution | 0.30 |
| 1,2-pentanediol (Hydrolite ® 5) | 0.30 |
| Triethylene Glycol. Imidazolidinyl | 0.30 |
| Urea. Methylparaben. Propylparaben. | |
| Dehydroacetic Acid | |
| Composition 1 | 0.20 |

TABLE F26
Cream (Amounts in % b.w.)

| Ingredients | Amount |
|---|---|
| Paraffin oil | 10.00 |
| Ozokerite | 4.00 |
| Vaseline | 4.00 |
| Vegetable oil | 10.00 |
| Wool wax alcohol | 2.00 |
| Aluminum stearate | 0.40 |
| 1,2-pentanediol (Hydrolite ® 5) | 2.00 |
| Phenoxyethanol | 0.50 |
| Composition 1 | 0.50 |
| Water | ad 100.00 |

TABLE F27
Cream (Amounts in % b.w.)

| Ingredients | INCI | Amount |
|---|---|---|
| Dracorin ® CE | Glyceryl Stearate Citrate | 1.00 |
| Lanette ® O | Cetearyl Alcohol | 2.00 |
| Cutina ® GMS-V | Glyceryl Stearate | 1.00 |
| Tegosoft ® MM | Myristyl Myristate | 1.00 |
| Xiameter ® PMX-0246. | Cyclohexasiloxane (and) | 0.50 |
| Cyclosiloxane | Cyclopentasiloxane | |
| Dragoxat ® 89 | Ethylhexyl Isononanoate | 2.00 |
| PCL-Liquid 100 | Cetearyl Ethylhexanoate | 4.00 |
| Neutral Oil | Caprylic/Capric Triglyceride | 4.00 |
| Carbopol ® Ultrez 21 | Acrylates/C10-30 Alkyl | 0.20 |
| | Acrylate Crosspolymer | |
| Keltrol ® CG-T | Xanthan Gum | 0.10 |
| Water | Water (Aqua) | Ad 100 |
| Glycerol 99.5 P. | Glycerol | 3.00 |
| Propylene Glycol -1.2 | Propylene Glycol | 2.00 |
| 99 P GC | | |
| Sodium Benzoate | Sodium Benzoate | 0.10 |
| Sodium Hydroxide | Sodium Hydroxide | 0.50 |
| 10% solution | | |
| Hydrolite ® 5 | 1,2-pentanediol | 0.30 |
| Euxyl ® K702 | Dehydroacetic Acid. | 0.30 |
| | Benzoic Acid. Phenoxy- | |
| | ethanol.Polyaminopropyl | |
| | Biguanide. Ethylhexylglycerin | |
| Composition 1 | | 0.70 |

TABLE F28
Hand and body cream (Amounts in % b.w.)

| Ingredients | INCI | Amount |
|---|---|---|
| Dracorin ® GOC | Glyceryl Oleate Citrate. | 2.00 |
| | Caprylic/Capric Triglyceride | |
| PCL-Solid | Stearyl Heptanoate. Stearyl Caprylate | 2.50 |
| Lanette ® O | Cetearyl Alcohol | 1.50 |
| Cutina ® GMS-V | Glyceryl Stearate | 1.00 |
| Dragoxat ® 89 | Ethylhexyl Isononanoate | 3.00 |
| PCL-Liquid 100 | Cetearyl Ethylhexanoate | 7.00 |
| Isodragol ® | Triisononanoin | 4.00 |
| Xiameter ® PMX-0345 Cyclosiloxane | Cyclopentasiloxane (and) Cyclohexasiloxane | 0.50 |
| Water | Water (Aqua) | Ad 100 |
| Carbopol ® Ultrez 21 | Acrylates/C10-30 Alkyl Acrylate Crosspolymer | 0.20 |
| Keltrol ® CG-RD | Xanthan Gum | 0.10 |
| Glycerol 85 P. | Glycerol | 3.00 |
| DragoBetaGlucan | Water (Aqua). Butylene Glycol. Glycerol. | 1.50 |
| | Avena Sativa (Oat) Kernel Extract | |
| Potassium Sorbat | Potassium Sorbate | 0.10 |
| Sodium Hydroxide 10% solution | Sodium Hydroxide | 0.50 |
| Hydrolite ® 5 | 1,2-pentanediol | 0.20 |
| Euxyl ® K300 | Methyl-. Butyl-. Ethyl-. Propyl. | 0.30 |
| | Isobutylparaben. Phenoxyethanol. | |
| Composition 1 | | 0.30 |

TABLE F29
Face cream (Amounts in % b.w.)

| Ingredients | INCI | Amount |
|---|---|---|
| Emulsiphos ® | Potassium Cetyl Phosphate. | 1.50 |
| | Hydrogenated Palm Glycerides | |
| Cutina ® GMS-V | Glyceryl Stearate | 1.70 |
| Lanette ® O | Cetearyl Alcohol | 3.00 |
| Tegosoft ® MM | Myristyl Myristate | 1.00 |
| PCL-Liquid 100 | Cetearyl Ethylhexanoate | 1.00 |
| Isodragol ® | Triisononanoin | 3.00 |
| Dragoxat ® 89 | Ethylhexyl Isononanoate | 4.00 |
| Avocado Oil | Persea Gratissima (Avocado) Oil | 3.00 |
| Abil ® 350 | Dimethicone | 0.50 |
| Covi-ox ® T-70 | Tocopherol | 0.10 |
| Edeta ® BD | Disodium EDTA | 0.10 |
| Carbopol ® Ultrez 21 | Acrylates/C10-30 Alkyl Acrylate Crosspolymer | 0.30 |
| Keltrol ® CG-RD | Xanthan Gum | 0.150 |
| Water | Water (Aqua) | Ad 100 |
| Glycerol 99.5 P. | Glycerol | 4.00 |
| Propylene Glycol -1.2 99 P GC | Propylene Glycol | 3.00 |
| SymMatrix ® | Maltodextrin. Rubus Fruticosus (Blackberry) | 0.50 |
| | Leaf Extract | |
| Sodium Hydroxide 10% solution | Sodium Hydroxide | 0.50 |
| Hydrolite ® 5 | 1,2-pentanediol | 0.30 |
| Euxyl ® K712 | Sodium Benzoate. Potassium Sorbate | 0.20 |
| Composition 1 | | 0.60 |

TABLE F30
Moisturizing body care cream (Amounts in % b.w.)

| Ingredient | Amount |
|---|---|
| PEG-7 hydrogenated castor oil | 6.00 |
| Cetearyl ethyl hexanoate | 10.00 |
| Isopropyl myristate | 5.00 |
| Mineral oil | 7.00 |
| Shea Butter (Butyrospermum parkii) | 0.50 |
| Aluminum stearate | 0.50 |
| Magnesium stearate | 0.50 |
| Bisabolol | 0.20 |
| Quaternium-18-Hectorit | 0.70 |
| Dipropylene glycol | 5.0 |
| Magnesium sulfate | 0.70 |
| 1,2-pentanediol (Hydrolite ® 5) | 4.00 |
| Preservative (Phenoxyethanol) | 0.20 |
| Composition 1 | 0.40 |
| Aqua dem. | 58.90 |

TABLE F31
Anti-wrinkle cream (Amounts in % b.w.)

| Ingredients | Amount |
|---|---|
| Glyceryl Stearate Citrate | 1.00 |
| Glyceryl Laurate | 1.00 |
| Cetearyl Alcohol (and) Myristyl Myristate | 3.00 |
| Cetearyl Ethylhexanoate | 4.00 |
| Mineral oil | 4.00 |
| Cyclohexasiloxane | 0.50 |
| Acrylates/C10-30 Alkyl Acrylate Crosspolymer | 0.20 |
| Water | Ad 100 |
| 1.2-Hexanediol | 2.00 |
| Sodium Hydroxide 10% solution | 0.10 |
| Narcissus Tazetta Bulb Extract | 1.00 |
| 1,2-pentanediol (Hydrolite ® 5) | 0.30 |
| Composition 1 | 0.50 |

TABLE F32
Functional skin oil for disinfection (Amounts in % b.w.)

| Ingredients | INCI | Amount |
|---|---|---|
| Neutral Oil | Caprylic/Capric Triglyceride | Ad 100 |
| Sweet Almond Oil | Prunus Dulcis | 20.00 |
| Dragoxat ® 89 | Ethylhexyl Isononanoate | 4.00 |
| Isopropyl Palmitate | Isopropyl Palmitate | 6.00 |
| PCL-Liquid 100 | Cetearyl Ethylhexanoate | 15.00 |
| Dragosantol ® 100 | Bisabolol | 0.20 |
| Retinyl Acetate In Oil | Retinyl Acetate | 0.50 |
| (1 Mio. Ie/G) | | |
| Vitamin E Acetate | Tocopheryl Acetate | 0.50 |
| Covi-ox ® T-70 | Tocopherol | 0.10 |
| Hydrolite ® 5 | 1,2-pentanediol | 0.30 |
| Preservative | Methyl-. Butyl-. Ethyl-. Propylparaben | 0.30 |
| Composition 1 | | 0.40 |

TABLE F33
Septic wound cream (Amounts in % b.w.)

| Ingredients (INCI) | Amount |
|---|---|
| Sorbitan Isostearate. Hydrogenated Castor | 6.00 |
| Oil. Ceresin. Beeswax (Cera Alba) | |
| Petrolatum | 21.00 |
| Cera Alba | 5.00 |
| Cetearyl Alcohol | 7.00 |
| Prunus Dulcis | 7.00 |
| Lanolin | 5.00 |
| Paraffinum Liquidum | 12.00 |
| Water (Aqua) | Ad 100 |
| Panthenol | 7.00 |
| Magnesium Sulfate | 0.70 |
| 1,2-pentanediol (Hydrolite ® 5) | 1.00 |
| Tocopheryl Acetate | 1.00 |
| Octenidine dihydrochloride | 0.10 |
| Phenoxyethanol | 0.50 |
| Composition 1 | 0.50 |

TABLE F34
Moisturizing and disinfecting face mask (Amounts in % b.w.)

| Ingredients | INCI | Amount |
|---|---|---|
| Water | Water (Aqua) | Ad 100 |
| Stabileze QM | PVM/Ma Decadiene Crosspolymer | 0.50 |
| Biotive ® L-Arginine | Arginine | 0.75 |
| Actipone ® Laminaria Saccharina GW | Glycerol. Water (Aqua). Laminaria Saccharina | 1.00 |
| | Extract | |
| Extrapone ® Cucumber | Water (Aqua). Propylene Glycol. Cucumis Sativus | 1.00 |
| | (Cucumber) Juice | |
| Glycerol 99.5 P. | Glycerol | 7.00 |
| Neo Actipone ® Soap Nutshell | Sapindus Mukurossi Peel Extract | 0.50 |
| Colour I | Colour | 0.01 |
| Hydrolite ® 5 | Pentylene Glycol | 5.00 |
| Solubilizer | PEG-40 Hydrogenated Castor Oil. Trideceth-9. | 0.60 |
| | Water (Aqua) | |
| Hydrolite ® 5 | 1,2-pentanediol | 0.08 |
| Preservative | Phenoxyethanol | 0.40 |
| Composition 1 | | 0.40 |

TABLE F35
Sprayable disinfecting gel (Amounts in % b.w.)

| Ingredients | INCI | Amount |
|---|---|---|
| Water | Water (Aqua) | Ad 100 |
| Stabileze QM | PVM/Ma Decadiene Crosspolymer | 0.25 |
| Sodium Hydroxide 10% solution | Sodium Hydroxide | 0.40 |
| Coffein pure | Caffeine | 0.50 |
| Extrapone ® Horse Chestnut | Propylene Glycol. Water (Aqua). Glucose. Aesculus | 1.00 |
| | Hippocastanum (Horse Chestnut) Seed Extract. | |
| | Lactic Acid | |
| Hydrolite ® 5 | 1,2-pentanediol | 3.00 |
| 1.3 Butylene Glycol | Butylene Glycol | 5.00 |
| Biotive ® Esculin Sesquihydrate | Esculin | 0.30 |
| Ethanol 96% | Alcohol Denat. | 10.00 |
| Solubilizer | PEG-40 Hydrogenated Castor Oil. Trideceth-9. | 0.50 |
| | Water (Aqua) | |
| Preservative | Phenoxyethanol | 0.70 |
| Composition 1 | | 0.30 |

TABLE F36
Mineral wash and cleaning gel (Amounts in % b.w.)

| Ingredients | INCI | Amount |
|---|---|---|
| Water | Water (Aqua) | Ad 100 |
| Pionier ® NP 37 G | Sodium Carbomer | 1.50 |
| SymSol ® PF-3 | Water (Aqua). Pentylene Glycol. Sodium Lauryl | 5.00 |
| | Sulfoacetate. SodiumOleoyl Sarcosinate. Sodium | |
| | Chloride. Disodium Sulfoacetate. SodiumOleate. | |
| | Sodium Sulfate | |
| Hydroviton ® 24 | Water (Aqua). Pentylene Glycol. Glycerol. Sodium | 1.00 |
| | Lactate. Lactic Acid. Serine. Urea. Sorbitol. Sodium | |
| | Chloride. Allantoin | |
| Extrapone ® Silk GW | Water (Aqua). Glycerol. Hydrolyzed Silk | 1.00 |
| Hydrolite ® 5 Green | Hydrolite ® 5 Green | 4.00 |
| Actipearls Red Star # DH10402/6 | Water (Aqua). Propylene Glycol. Algin. Gellan | 1.00 |
| | Gum. Xanthan Gum. CalciumChloride. CI 12490 | |
| | (Pigment Red 5). Mica (CI 77019). Titanium | |
| | Dioxide (CI 77891) | |
| 1,2-pentanediol (Hydrolite ® 5) | 1,2-pentanediol | 0.50 |
| 3-Phenylpropanol | | 0.70 |
| Composition 1 | | 0.30 |

TABLE F37
Anti-acne wash (Amounts in % b.w.)

| Ingredients (INCI) | Amount |
|---|---|
| Water (Aqua) | 45.70 |
| Polyquaternium-7 | 0.50 |
| Cocamidopropyl Betaine 9.000 | 9.00 |
| Coco Glucoside 2.000 | 2.00 |
| Polysorbate 80. Glycerol. Gossypium Herbaceum. | 1.00 |
| (Cotton) Seed Oil. Water (Aqua) | |
| Trideceth-9. PEG-5 Ethylhexanoate. Water (Aqua) | 1.00 |
| Glycereth-90 Isostearate. Laureth-2 | 0.50 |
| Sodium Laureth Sulfate 37.000 | 37.00 |
| Glycerol. Triticum Vulgare (Wheat) Gluten. | 1.00 |
| Water (Aqua) | |
| Sodium Chloride | 0.30 |
| 1,2-pentanediol (Hydrolite ® 5) | 1.00 |
| Composition 1 | 0.50 |

TABLE F38
Cosmetic sun protection composition (Amounts in % b.w.)

| Ingredient | Amount |
|---|---|
| Ethylhexyl cinnamic acid | 7.50 |
| Benzophenon-3 | 2.00 |
| Polyglyceryl dimer soyate | 0.80 |
| Sorbitane stearate | 1.00 |
| Tocopheryl acetate | 0.50 |
| Glyceryl stearate. PEG-100 Stearate | 3.00 |
| PEG-40. hydrogenated castor oil | 1.00 |
| Titanium dioxide. aluminum oxide hydrate. | 3.00 |
| Dimethicon/Methicon Copolymer | |
| Butyrospermum parkii (Shea Butter) | 1.00 |
| C12-15 alkyl benzoate | 6.50 |
| Butylene glycol | 5.00 |
| Xanthan gum | 0.30 |
| Disodium EDTA | 0.10 |
| Allantoin | 0.10 |
| Polyacryl amide. C₁₃₋₁₄ isoparaffin. Laureth-7 | 1.00 |
| 1,2-pentanediol (Hydrolite ® 5) | 5.00 |
| 4-t Butylcyclohexanol | 1.00 |
| Preservatives (Methyl-. Butyl-. Ethyl-. | 0.30 |
| Propylparaben. Phenoxyethanol) | |
| Composition 1 | 0.60 |
| Aqua dem. | Ad 100 |

TABLE F39
Sun protection spray (Amounts in % b.w.)

| Ingredients | INCI | Amount |
|---|---|---|
| Water. demineralized | Water (aqua) | 69.50 |
| Glycerol | Glycerol | 4.00 |
| 1.3 butylene glycol | Butylene glycol | 5.00 |
| D-Panthenol | Panthenol | 0.50 |
| Lara Care A-200 | Galactoarabinan | 0.25 |
| Baysilone oil M 10 | Dimethicone | 1.00 |
| Edeta BD | Disodium EDTA | 0.10 |
| Copherol 1250 | Tocopheryl acetate | 0.50 |
| Cetiol OE | Dicaprylyl ether | 3.00 |
| Neo Heliopan ® HMS | Homosalate | 5.00 |
| Neo Heliopan ® AV | Ethylhexyl methoxycinnamate | 6.00 |
| Neo Heliopan ® 357 | Butyl methoxydibenzoylmethane | 1.00 |
| Corapan TQ | Diethylhexylnaphthalate | 2.00 |
| Alpha Bisabolol | Bisabolol | 0.10 |
| Pemulen TR-2 | Acrylates/C10-30 alkyl acrylate | 0.25 |
| | crosspolymer | |
| NaOH. 10% | Sodium hydroxide | 0.60 |
| Hydrolite ® 5 | 1,2-pentanediol | 0.20 |
| Phenoxyethanol | Phenoxyethanol | 0.40 |
| Solbrol M | Methylparaben | 0.10 |
| Solbrol P | Propylparaben | 0.10 |
| Composition 1 | | 0.50 |

TABLE F40
Sunscreen spray O/W. SPE 15-20 (Amounts in % b.w.)

| Ingredients | INCI | Amount |
|---|---|---|
| Dracorin ® GOC | Glyceryl Oleate Citrate. Caprylic/Capric | 2.00 |
| | Triglyceride | |
| Corapan ® TQ | Diethylhexyl 2.6-Naphthalate | 3.00 |
| Neo Heliopan ® HMS | Homosalate | 7.00 |
| Neo Heliopan ® OS | Ethylhexyl Salicylate | 5.00 |
| Neo Heliopan ® 357 | Butyl Methoxydibenzoylmethane | 3.00 |
| Isoadipate | Diisopropyl Adipate | 6.00 |
| Baysilone ® Oil M10 | Dimethicone | 1.00 |
| Edeta ® BD | Disodium EDTA | 0.10 |
| Vitamin E Acetate | Tocopheryl Acetate | 0.50 |
| Dragosantol ® 100 | Bisabolol | 0.10 |
| Pemulen ® TR-2 | Acrylates/C10-30 Alkyl Acrylate Crosspolymer | 0.25 |
| Water | Water (Aqua) | Ad 100 |
| Glycerol 99.5 P. | Glycerol | 4.00 |
| Butylene Glycol | Butylene Glycol | 5.00 |
| Neo Heliopan ® Hydro (103089). | Phenylbenzimidazole Sulfonic Acid | 8.00 |
| used as 25% aq. solution | | |
| Biotive ® L-Arginine | Arginine | 0.55 |
| Hydrolite ® 5 | 1,2-pentanediol | 0.40 |
| Sobrol M | Methylparaben | 0.30 |
| Composition 1 | | 0.60 |

TABLE F41
Sun protection soft cream (W/O). SPF 40 (Amounts in % b.w.)

| Ingredients | INCI | Amount |
|---|---|---|
| Dehymuls PGPH | Polyglyceryl-2 dipolyhydroxystearate | 5.00 |
| Copherol 1250 | Tocopheryl acetate | 0.50 |
| Permulgin 3220 | Ozocerite | 0.50 |
| Zinc stearate | Zinc stearate | 0.50 |
| Tegosoft TN | C12-15 Alkyl benzoate | 10.00 |
| Neo Heliopan ® E1000 | Isoamyl-p-methoxycinnamate | 2.00 |
| Neo Heliopan ® 303 | Octocrylene | 5.00 |
| Neo Heliopan ® MBC | 4-Methylbenzylidene camphor | 3.00 |
| Zinc oxide. neutral | Zinc oxide | 5.00 |
| Water. distilled | Water (aqua) | Add 100 |
| EDETA BD | Disodium EDTA | 0.10 |
| Glycerol | Glycerol | 4.00 |
| Magnesium sulfate | Magnesium sulfate | 0.50 |
| Hydrolite ® 5 | 1,2-pentanediol | 0.30 |
| Symdiol ® 68 | 1.2-Hexanediol. Caprylylglycol | 0.30 |
| Composition 1 | | 0.80 |

TABLE F42
Sun protection milk (W/O) (Amounts in % b.w.)

| Ingredients | INCI | Amount |
|---|---|---|
| Dehymuls PGPH | Polyglyceryl-2 dipolyhydroxystearate | 3.00 |
| Beeswax 8100 | Beeswax | 1.00 |
| Monomuls 90-0-18 | Glyceryl oleate | 1.00 |
| Zinc stearate | Zinc stearate | 1.00 |
| Cetiol SN | Cetearyl isononanoate | 5.00 |
| Cetiol OE | Dicaprylyl ether | 5.00 |
| Tegosoft TN | C12-15 alkyl benzoate | 4.00 |
| Vitamin E | Tocopherol | 0.50 |
| Neo Heliopan ® OS | Ethylhexyl salicylate | 5.00 |
| Neo Heliopan ® AV | Ethylhexyl methoxycinnamate | 7.50 |
| Uvinul ® T150 | Ethylhexyl triazone | 1.50 |
| Water. distilled | Water (Aqua) | To 100 |
| Trilon BD | Disodium EDTA | 0.10 |
| Glycerol | Glycerol | 5.00 |
| Neo Heliopan ® | Disodium phenyl dibenzimidazole | 15.00 |
| AP 10% solution. | tetrasulfonate | |
| neutralized with NaOH | | |
| Hydrolite ® 5 | 1,2-pentanediol | 0.25 |
| Alpha bisabolol | Bisabolol | 0.10 |
| SymOcide ® PT | Phenoxyethanol. Tropolone | 0.25 |
| Composition 1 | | 0.25 |

TABLE F43
After sun gel (Amounts in % b.w.)

| Ingredients | INCI | Amount |
|---|---|---|
| SymSol ® PF-3 | Water (Aqua). Pentylene Glycol. Sodium Lauryl | 3.000 |
| | Sulfoacetate. SodiumOleoyl Sarcosinate. Sodium | |
| | Chloride. Disodium Sulfoacetate. SodiumOleate. | |
| | Sodium Sulfate | |
| Glycerol 99.5 P. | Glycerol | 5.000 |
| SymHelios ® 1031 | Benzylidene Dimethoxydimethylin danone | 0.100 |
| Water | Water (Aqua) | Ad 100 |
| Pemulen ® TR-2 | Acrylates/C10-30 Alkyl Acrylate Crosspolymer | 1.000 |
| D-Panthenol 75 W | Panthenol | 0.500 |
| SymFinity ® 1298 | Echinacea Purpurea Extract | 0.100 |
| Extrapone ® Pearl GW | Water (Aqua). Glycerol. Hydrolyzed Pearl. | 1.000 |
| | Xanthan Gum | |
| Sodium Hydroxide 10% solution | Sodium Hydroxide | 2.500 |
| Ethanol 96% | Alcohol Denat. | 15.000 |
| Hydrolite ® 5 | 1,2-pentanediol | 0.20 |
| SymOcide ® PS | Phenoxyethanol. 1.2-Hexanediol. Decyleneglycol | 0.50 |
| Composition 1 | | 0.50 |

TABLE F44
After sun lotion (Amounts in % b.w.)

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| 1.2-Hexanediol | 0.60 |
| Composition 1 | 0.30 |
| 1,2-pentanediol (Hydrolite ® 5) | 4.0 |
| Aqua dem. | Ad 100 |
| Triethanolamine | 0.2 |

TABLE F45
Hair styling gel (Amounts in % b.w.)

| Ingredients | Amount |
|---|---|
| Water | Ad 100 |
| PVM/MA Decadiene Crosspolymer | 0.60 |
| PVP | 3.00 |
| Isocetyl Stearate | 4.00 |
| Ethylhexyl Methoxycinnamate | 0.50 |
| Aminomethyl Propanol | 0.40 |
| 1,2-pentanediol (Hydrolite ® 5) | 0.60 |
| SymDiol ® 68T (1.2-Hexanediol. | 0.30 |
| 1.2-Octanediol. Tropolone) | |
| Phenoxyethanol | 0.20 |
| Composition 1 | 0.40 |

TABLE F46
Silicone emulsion (Amounts in % b.w.)

| Ingredients | Amount |
|---|---|
| Potassium Cetyl Phosphate. Hydrogenated | 1.00 |
| Palm Glycerides | |
| Cyclohexasiloxane | 4.00 |
| Cetearyl Alcohol | 1.50 |
| Phenyl Trimethicone | 3.00 |
| Stearyl Heptanoate. Stearyl Caprylate | 3.00 |
| Dimethicone | 1.00 |
| Xanthan Gum | 0.20 |
| Isoamyl p-Methoxycinnamate | 5.00 |
| Water | Ad 100 |
| Methylpropanediol | 3.00 |
| 1,2-pentanediol (Hydrolite ® 5) | 0.30 |
| Diazolidinyl urea | 0.10 |
| Composition 1 | 0.50 |

TABLE F47
Microemulsion gel (Amounts in % b.w.)

| Ingredient | Amount |
|---|---|
| Glycerol isostearate | 1.80 |
| Octoxyglycerol | 1.00 |
| Ceteareth-15 | 5.20 |
| PEG-150 Distearate | 1.00 |
| Aluminium chlorohydrate | 5.00 |
| Isotridecylisononanoate | 3.30 |
| Cyclomethicone | 6.60 |
| 1,2-pentanediol (Hydrolite ® 5) | 0.70 |
| Euxyl ® K145 | 0.10 |
| (Methylchloroisothiazolinone. | |
| Methylisothiazlinone. Bronopol) | |
| Composition 1 | 0.40 |
| Water | Ad 100 |

TABLE F48
Air freshener in gel form (Amounts in % b.w.)

| Ingredient | Amount |
|---|---|
| Demineralised water | Ad 100 |
| Genugel ® X-6424 (carrageenan) | 2.00 |
| Arkopal ® N 100 or Tergitol ® | 3.50 |
| NP 10 (Emulsifer) | |
| 1,2-pentanediol (Hydrolite ® 5) | 0.60 |
| Preventol ® D 7 (5-chloro-2- | 0.10 |
| methyl-4-isothiazolin-3-one. 2- | |
| methyl-2H isothiazol-3-one) | |
| Composition 1 | 0.80 |

TABLE F49
Cleaner. APC liquid. alkaline
pH 8-10 (Amounts in % b.w.)

| Ingredients (INCI) | Amount |
|---|---|
| Aqua | 59.06 |
| Tri Sodium Citrate Dihydrate | 3.00 |
| Sodium Laureth Sulfate | 30.00 |
| Trideceth-9 | 5.00 |
| Ethanol | 2.00 |
| Citric Acid 10% solution | 0.24 |
| 1,2-pentanediol (Hydrolite ® 5) | 0.50 |
| Mixture of 5-Chloro-2-methyl- | 0.10 |
| 2H-isothiazol-3-one and 2- | |
| Methyl-2H-isothiazol-3-one | |
| Composition 1 | 0.30 |

TABLE F50
Fabric softener (Amounts in % b.w.)

| Ingredients (INCI) | Amount |
|---|---|
| Aqua | 72.10 |
| Dialkylester ammomium methosulfate | 16.60 |
| Polydimethylsiloxane | 0.30 |
| Magnesiumchloride | 10.00 |
| 1,2-pentanediol (Hydrolite ® 5) | 0.60 |
| Mixture of 5-Chloro-2-methyl-2H- | 0.10 |
| isothiazol-3-one and 2-Methyl-2H- | |
| isothiazol-3-one | |
| Composition 1 | 0.40 |

TABLE F51
Liquid detergent (Amounts in % b.w.)

| Ingredients | Amount |
|---|---|
| Deionized water | 39.60 |
| Optical brightener | 0.10 |
| Coconut fatty acids (C12-C18) | 7.50 |
| Potassium hydroxide 50% solution | 4.30 |
| Propane-1,2-diol | 5.00 |
| Fatty alcohols C12-C15. 8 EO | 12.00 |
| Na-salt of secondary alkyl | 17.00 |
| sulfonates (C13-C17) | |
| Triethanolamine | 2.00 |
| Trisodium citrate dihydrate | 5.00 |
| Dequest 2066 Diethylenetriamine | 3.00 |
| penta(methylene phosphonic acid) | |
| Ethanol | 3.00 |
| Enzymes | 0.70 |
| 1,2-pentanediol (Hydrolite ® 5) | 0.50 |
| Composition 1 | 1.00 |

TABLE F52
Liquid detergent concentrate
(Amounts in % b.w.)

| Ingredients | Amount |
|---|---|
| Deionized water | 12.9 |
| Coconut fatty acids (C12-C18) | 10.0 |
| Fatty alcohols C12-C15. 8 EO | 26.0 |
| Na-salt of secondary alkyl | 26.5 |
| sulfonates (C13-C17) | |
| Triethanol amine | 8.5 |
| Na-salt of fatty alcohol sulfates | 3.0 |
| C12-C14 | |
| Ethanol | 5.5 |
| Urea | 4.5 |
| Enzymes | 0.9 |
| Citric acid | 1.0 |
| 1,2-pentanediol (Hydrolite ® 5) | 0.7 |
| Composition 1 | 0.8 |

TABLE F53
Toilet cleaner (Amounts in % b.w.)

| Ingredients | Amount |
|---|---|
| Water | 93.0 |
| Kelzan ASX-T | 0.5 |
| Parafin sulfonate. sodium salt | 1.0 |
| Citric acid | 5.0 |
| Colorant (FD & C Yellow No. 6) | 0.1 |
| 1,2-pentanediol (Hydrolite ® 5) | 0.3 |
| Preservative | 0.05 |
| (Benzisothiazolinone. Glutaral) | |
| Composition 1 | 0.6 |

TABLE F54
Dish washing concentrate
(Amounts in % b.w.)

| Ingredients | Amount |
|---|---|
| Sodium laurylsulfate | 31.0 |
| Propane-1.2-diole | 6.0 |
| Ethyl alcohol 96% | 7.0 |
| Palm tree glucosides | 6.0 |
| Coco betaine | 18.0 |
| 1,2-pentanediol | 0.4 |
| (Hydrolite ® 5) | |
| Composition 1 | 0.5 |
| Water | 31.6 |

TABLE F55
Dish washing concentrate
(Amounts in % b.w.)

| Ingredients | Amount |
|---|---|
| Palm tree glucosides | 4.0 |
| Sodium lauryl sulfate | 45.0 |
| Coco betaine | 8.0 |
| Ethyl alcohol 96% | 1.0 |
| Colorant (C.I. | 0.05 |
| Pigment Blue 15) | |
| 1,2-pentanediol | 0.2 |
| (Hydrolite ® 5) | |
| Composition 1 | 0.7 |
| Water | Ad 100 |

TABLE F56
Solution for wet wipes (Amounts in % b.w.)

| Ingredients | INCI | Amount |
|---|---|---|
| SymSol ® PF-3 | Water (Aqua). Pentylene Glycol. | 2.00 |
| | Sodium Lauryl Sulfoacetate. | |
| | SodiumOleoyl Sarcosinate. Sodium | |
| | Chloride. Disodium Sulfoacetate. | |
| | SodiumOleate. Sodium Sulfate | |
| Dragosantol ® 100 | Bisabolol | 0.10 |
| Glycerol 99.5 P. | Glycerol | 5.00 |
| Water | Water (Aqua) | Ad 100 |
| Hydrolite ® 5 | 1,2-pentanediol | 5.00 |
| D-Panthenol 75 W | Panthenol | 0.80 |
| DragoCalm ® | Water (Aqua). Glycerol. Avena | 1.00 |
| | Sativa (Oat) Kernel Extract | |
| Witch Hazel- | Hamamelis Virginiana (Witch | 1.00 |
| Distillate | Hazel) Water. Water (Aqua). | |
| | Alcohol | |
| Allplant Essence ® | Pelargonium Graveolens Flower/ | 1.00 |
| Org. Rose | Leaf/Stem Water | |
| Geranium P | | |
| Preservative | Phenoxyethanol | 0.30 |
| Composition 1 | | 0.50 |

TABLE F57
Chewing gum, free of sugar;
all amounts in % b.w.

| Composition | A | B | C |
|---|---|---|---|
| Gum base | 30.00 | 30.00 | 30.00 |
| Sorbit, powdered | 40.00 | 40.00 | 40.00 |
| Isomalt, powdered | 9.50 | 9.50 | 9.50 |
| Xylitol | 2.00 | 2.00 | 2.00 |
| Mannit D | 3.00 | 3.00 | 3.00 |
| Aspartame | 0.10 | 0.10 | 0.10 |
| Acesulfam K | 0.10 | 0.10 | 0.10 |
| Emulgum/ | 0.30 | 0.30 | 0.30 |
| Plasticizing agent | | | |
| Sorbitol (70% water) | 13.00 | 13.00 | 13.00 |
| Glycerol | 1.00 | 1.00 | 1.00 |
| Composition 1 | 0.01 | 0.01 | 0.01 |
| Total | | 100 | |

TABLE F58
Tooth paste; all amounts in % b.w.

| Composition | A | B | C |
|---|---|---|---|
| Deionised water | 36.39 | 36.39 | 36.39 |
| Glycerol | 20.00 | 20.00 | 20.00 |
| Solbrol M (sodium salt) | 0.15 | 0.15 | 0.15 |
| Sodium monofluor | 0.76 | 0.76 | 0.76 |
| phosphate | | | |
| Saccharin | 0.20 | 0.20 | 0.20 |
| Dicalciumphosphate | 36.00 | 36.00 | 36.00 |
| dihydrate | | | |
| Aerosil 200 | 3.00 | 3.00 | 3.00 |
| Sodium carboxymethyl | 1.20 | 1.20 | 1.20 |
| cellulose | | | |
| Sodium lauryl sulfate | 1.30 | 1.30 | 1.30 |
| Composition 1 | 0.01 | 0.01 | 0.01 |
| Total | | 100 | |

TABLE F59
Mouth wash concentrate; all amounts in % b.w.

| Composition | A | B | C |
|---|---|---|---|
| Ethanol 96% | 42.00 | 42.00 | 42.00 |
| Cremophor RH 455 | 5.00 | 5.00 | 5.00 |
| Deionised water | 50.67 | 50.67 | 50.67 |
| Allantoin | 0.20 | 0.20 | 0.20 |
| Sodium saccharin 450 | 0.10 | 0.10 | 0.10 |
| Colour L-Blue 5000 | 0.03 | 0.03 | 0.03 |
| (1% in Wasser) | | | |
| Composition 1 | 0.01 | 0.01 | 0.01 |
| Total | | 100 | |

TABLE F60
Hard boiled candy, sugar-free;
all amounts in % b.w.

| Composition | A | B | C |
|---|---|---|---|
| Water | 2.24 | 2.24 | 2.24 |
| Isomalt | 94.98 | 94.98 | 94.98 |
| Xylitol | 2.40 | 2.40 | 2.40 |
| Sucralose | 0.03 | 0.03 | 0.03 |
| Acesulfame K | 0.050 | 0.050 | 0.050 |
| Citric acid | 0.050 | 0.050 | 0.050 |
| Composition 1 | 0.01 | 0.01 | 0.01 |
| Total | | 100 | |

TABLE F61
Hard boiled candy; all amounts in % b.w.

| Composition | A | B | C |
|---|---|---|---|
| Water | 2.75 | 2.24 | 2.24 |
| Glucose | 60.1 | 94.98 | 94.98 |
| Glucose syrup | 36.9 | 2.40 | 2.40 |
| Composition 1 | 0.01 | 0.01 | 0.01 |
| Total | | 100 | |

1. A composition comprising
(a) at least one substituted azacycle according to formula (I), a salt or a solvate thereof 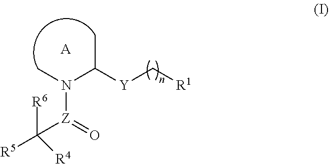
wherein
n is 0 or 1 and R1 is selected from
(i) halogen,
(ii) C6-C10 aryl,
(iii) C5-C10 mono- or bicyclic heteroaryl wherein up to 2 C-atoms are replaced by at least one hetero atom independently selected from sulfur, nitrogen, and oxygen,
(iv) C6-C10 aryl substituted with up to four substituents independently selected from the group consisting of halogen; OH; C═N; NO2; C1-C6 alky; C1-C6 alkyl comprising up to 5 halogen atoms; C1-C3 alkyl comprising up to 3 OH groups; C2-C6 alkenyl; C1-C6 alkoxy; C1—C6 alkoxy comprising up to 3 halogen atoms; C1-C3 alkoxy C1-C3 alkyl; C3-C7 cycloalkyl; —C(O)R10 wherein R10 is selected from C1-C3 alkyl; —OC(O)R11 wherein R11 is selected from H, and C1-C3 alkyl; —C(O)O—R12 wherein R12 is selected from hydrogen and C1-C3 alkyl; —(CH2)mN(R13)R14 wherein m is 0 or 1, R13 is selected from hydrogen, C1-C3-alkyl, and —SOR15 wherein R15 is C1-C3-alkyl, and R14 is selected from hydrogen, C1-C3-alkyl, and —SO2R16 wherein R16 is C1-C3-alkyl, or wherein R13 and 14 form together with the N atom to which they are attached morpholine, thiomorpholine, or 1,1-dioxothiomorpholine; —SR17 wherein R17 is selected from hydrogen and C1-C3 alkyl; and —S(O)2R18 wherein R18 is selected from hydrogen and C1-C3 alkyl; with the proviso that when the aryl ring is substituted with two or more substituents, two substituents may form a cyclic ring together with the carbon atoms to which they are attached, and
(v) C5-C10 mono- or bicyclic heteroaryl wherein up to 2 C-atoms are replaced by a hetero atoms independently selected from sulfur, nitrogen, and oxygen, substituted with up to four substituents selected from the group consisting of halogen; OH (hydroxyl); C═N (cyano); NO2 (nitro); C1-C6 alky; C1-C6 alky comprising up to 5 halogen atoms; C2-C6 alkenyl; C1-C6 alkoxy; C1-C6 alkoxy comprising up to 3 halogen atoms; C1-C3 alkoxy C1-C3 alkyl; C3-C7 cycloalkyl; —C(O)R20 wherein R20 is selected from C1-C3 alkyl; —OC(O)R21 wherein R21 is selected from H, and C1-C3 alkyl; —C(O)O—R22 wherein R22 is selected from hydrogen and C1-C3 alkyl; —(CH2)mN(R23)R24 wherein m is 0 or 1, R23 is selected from hydrogen, C—C alkyl, and —SOR25 wherein R25 24 1 3 2 is C1-C3 alkyl, and R is selected from hydrogen, C 26 26 1-C3 alkyl, and —SO2R wherein R is C1-C3 alkyl, or wherein R23 and R24 form together with the N atom to which they are attached morpholine, thiomorpholine, or 1,1-dioxothiomorpholine; —SR27 wherein R27 is selected from hydrogen and C1-C3 alkyl; and —S(O)2R28 wherein R28 is selected from hydrogen and C1-C3 alkyl; Y is a monocyclic or bicyclic, unsaturated or aromatic heterocyclic ring comprising one, two, three or four heteroatoms independently selected from nitrogen, sulfur and oxygen, wherein the ring is optionally mono-, di-, or tri-substituted with a group selected from halogen, methyl, ethyl, —N(R40)R41, —CON(R40)R41, —[CR40R41]—C(O)OR40, —C 40 40 41 p (O)R, and —SO2N(R)R wherein R40 and R41 are independently selected from hydrogen and C1-C4 alkyl, and p is 0, 1, or 2; A is a 5 to 7 membered heterocyclic ring optionally comprising one additional hetero atom selected from oxygen and sulfur, wherein the heterocyclic ring A is optionally substituted by one or two groups selected from —OH and ═O; Z is either C, S or S(O);
and R4, R5 and R6 form together with the carbon atom to which they are attached a hydrocarbon group optionally comprising up to five hetero atoms selected from O, N, S, and F;
(b) at least one physiological cooling or warming agent, and/or
(c) at least one aroma or flavoring compound, and/or
(d) at least one fragrance,
wherein the compounds are present in a ratio by weight (a):(b+c+d) of from about 1:1,500,000:1 to about 1:1.

2. The composition of claim 1₁ comprising the ingredients in a ratio by weight (a):(b+c+d) of from about 1:1,500,000 to about 1:10.

3. The composition of claim 1₁ comprising the ingredients in a ratio by weight (a):(b+c+d) of from about 1:100,000 to about 1:100.

4. The composition of claim 1, comprising the compounds in a ratio by weight (a):(b+c+d) of from about 1:40,000 to about 1:500.

5. The composition of claim 1, wherein compound (a) is selected from the group consisting of
(2-(4-phenyl-1H-imidazol-2-yl)piperidin-1-yl)(1,2,3-thiadiazol-5-yl)methanone,
(2S)-2-methyl-1-[2-[5-(p-tolyl)-1H-imidazol-2-yl]-1-piperidyl]butan-1-one,
2-methyl-1-[2-[5-(p-tolyl)-1H-imidazol-2-yl]-1-piperidyl]butan-1-one,
2-methylsulfanyl-1-[(1R)-2-[5-(p-tolyl)-1H-imidazol-2-yl]-1-piperidyl]-propan-1-one,
(2-(4-phenyl-1H-imidazol-2-yl)piperidin-1-yl)(thietan-3-yl)methanone,
(2,2-dimethylcyclopropyl)(2-(4-phenyl-1H-imidazol-2-yl)piperidin-1-yl)methanone,
(2-ethoxycyclopropyl)(2-(4-phenyl-1H-imidazol-2-yl)piperidin-1-yl)methanone,
(2-methoxycyclopropyl)(2-(4-phenyl-1H-imidazol-2-yl)piperidin-1-yl)methanone,
(2-methylcyclopropyl)(2-(4-phenyl-1H-imidazol-2-yl)piperidin-1-yl)methanone,
(2S)-2-methyl-1-(2-(5-(p-tolyl)-1H-imidazol-2-yl)piperidin-1-yl)butan-1-one,
(5-methyltetrahydrofuran-3-yl)(2-(4-phenyl-1H-imidazol-2-yl)piperidin-1yl)methanone,
(E)-1-(2-(5-(3,4-difluorophenyl)-1H-imidazol-2-yl)piperidin-1-yl)-2-methylbut-2-en-1-one,
(E)-2-methyl-1-(2-(5-(p-tolyl)-1H-imidazol-2-yl)piperidin-1-yl)but-2-en-1-one,
(E)-2-methyl-1-(2-(5-(p-tolyl)-1H-imidazol-2-yl)piperidin-1-yl)pent-3-en-1-one,
(R)-2-(methylthio)-1-((R)-2-(5-(p-tolyl)-1H-imidazol-2-yl)piperidin-1-yl)propan-1-one,
(S)-2-(methylthio)-1-((R)-2-(5-(p-tolyl)-1H-imidazol-2-yl)piperidin-1-yl)propan-1-one,
(S)-2-(methylthio)-1-((S)-2-(5-(p-tolyl)-1H-imidazol-2-yl)piperidin-1-yl)propan-1-one,
(S)-2-methyl-1-((R)-2-(5-(p-tolyl)-1H-imidazol-2-yl)piperidin-1-yl)butan-1-one,
(tetrahydrofuran-3-yl)(2-(5-(p-tolyl)-1H-imidazol-2-yl)piperidin-1-yl)methanone,
1-(2-(1H-benzo[d]imidazol-2-yl)piperidin-1-yl)-2-(methylthio)propan-1-one,
1-(2-(4-(1-methyl-1H-pyrazol-4-yl)-1H-imidazol-2-yl)piperidin-1-yl)-2-(methylthio)propan-1-one,
1-(2-(4-(2,2-difluorobenzo[d][1,3]dioxol-5-yl)-1H-imidazol-2-yl)piperidin-1-yl)-2-(methylthio)propan-1-one,
1-(2-(4-(2,3-dihydrobenzofuran-5-yl)-1H-imidazol-2-yl)piperidin-1-yl)-2-(methylthio)propan-1-one,
1-(2-(4-(2,3-dihydrobenzofuran-6-yl)-1H-imidazol-2-yl)piperidin-1-yl)-2-(methylthio)propan-1-one,
1-(2-(4-(2,5-dimethylphenyl)-1H-imidazol-2-yl)piperidin-1-yl)-2-(methylthio)propan-1-one,
1-(2-(4-(2-chlorophenyl)-1H-imidazol-2-yl)piperidin-1-yl)-2-(methylthio)propan-1-one,
1-(2-(4-(2-cyclopropyloxazol-5-yl)-1H-imidazol-2-yl)piperidin-1-yl)-2-(methylthio)propan-1-one,
1-(2-(4-(2-fluorophenyl)-1H-imidazol-2-yl)piperidin-1-yl)-2-(methylthio)propan-1-one,
1-(2-(4-(2-hydroxyphenyl)-1H-imidazol-2-yl)piperidin-1-yl)-2-(methylthio)propan-1-one,
(2-(4-(3-((dimethylamino)methyl)phenyl)-1H-imidazol-2-yl)piperidin-1-yl)-2-(methylthio)propan-1-one,
1-(2-(4-(3,5-difluorophenyl)-1H-imidazol-2-yl)piperidin-1-yl)-2-(methylthio)propan-1-one,
1-(2-(4-(3,5-dimethylisoxazol-4-yl)-1H-imidazol-2-yl)piperidin-1-yl)-2-(methylthio)propan-1-one,
1-(2-(4-(3-chloro-4-methylphenyl)-1H-imidazol-2-yl)piperidin-1-yl)-2-(methylthio)propan-1-one,
1-(2-(4-(3-methylfuran-2-yl)-1H-imidazol-2-yl)piperidin-1-yl)-2-(methylthio)propan-1-one,
1-(2-(4-(4-((1,1-dioxidothiomorpholino)methyl)phenyl)-1H-imidazol-2-yl)piperidin-1-yl)-2-(methylthio)propan-1-one,
1-(2-(4-(4-(1-hydroxyethyl)phenyl)-1H-imidazol-2-yl)piperidin-1-yl)-2-(methylthio)propan-1-one,
1-(2-(4-(4-(difluoromethoxy)-3-fluorophenyl)-1H-imidazol-2-yl)piperidin-1-yl)-2-(methylthio)propan-1-one,
1-(2-(4-(4-(difluoromethyl)thiophen-3-yl)-1H-imidazol-2-yl)piperidin-1-yl)-2-(methylthio)propan-1-one,
1-(2-(4-(4-(methylsulfonyl)phenyl)-1H-imidazol-2-yl)piperidin-1-yl)-2-(methylthio)propan-1-one,
1-(2-(4-(4-(tert-butyl)phenyl)-1H-imidazol-2-yl)piperidin-1-yl)-2-(methylthio)propan-1-one,
1-(2-(4-(4-cyclobutylphenyl)-1H-imidazol-2-yl)piperidin-1-yl)-2-(methylthio)propan-1-one,
1-(2-(4-(4-cyclopropyl-2-fluorophenyl)-1H-imidazol-2-yl)piperidin-1-yl)-2-(methylthio)propan-1-one,
1-(2-(4-(4-fluorophenyl)-1H-imidazol-2-yl)piperidin-1-yl)-2-(methylthio)propan-1-one,
1-(2-(4-(4-hydroxyphenyl)-1H-imidazol-2-yl)piperidin-1-yl)-2-(methylthio)propan-1-one,
1-(2-(4-(4-isopropylphenyl)-1H-imidazol-2-yl)piperidin-1-yl)-2-(methylthio)propan-1-one,
1-(2-(4-(4-methyl-3-(trifluoromethyl)phenyl)-1H-imidazol-2-yl)piperidin-1-yl)-2-(methylthio)propan-1-one,
1-(2-(4-(5-((dimethylamino)methyl)thiophen-2-yl)-1H-imidazol-2-yl)piperidin-1-yl)-2-(methylthio)propan-1-one,
1-(2-(4-(5-methylfuran-2-yl)-1H-imidazol-2-yl)piperidin-1-yl)-2-(methylthio)propan-1-one,
1-(2-(4-(5-methylpyridin-3-yl)-1H-imidazol-2-yl)piperidin-1-yl)-2-(methylthio)propan-1-one,
1-(2-(4-(6-methylpyridin-3-yl)-1H-imidazol-2-yl)piperidin-1-yl)-2-(methylthio)propan-1-one,
1-(2-(4-(benzo[b]thiophen-6-yl)-1H-imidazol-2-yl)piperidin-1-yl)-2-(methylthio)propan-1-one,
1-(2-(4-methyl-5-phenyl-1H-imidazol-2-yl)piperidin-1-yl)-2-(methylthio)propan-1-one,
1-(2-(4-phenyl-1H-imidazol-2-yl)piperidin-1-yl)-2-((trifluoromethyl)thio)ethan-1-one,
1-(2-(4-phenyl-1H-imidazol-2-yl)piperidin-1-yl)butan-1-one,
1-(2-(4-phenyl-1H-imidazol-2-yl)piperidin-1-yl)hexa-3,5-dien-1-one,
1-(2-(4-phenyl-1H-imidazol-2-yl)piperidin-1-yl)pent-4-en-1-one,
1-(2-(4-phenyl-1H-imidazol-2-yl)piperidin-1-yl)propan-1-one,
1-(2-(5-(2,3-difluorophenyl)-1H-imidazol-2-yl)piperidin-1-yl)-2-(methylthio)propan-1-one,
1-(2-(5-(2,4-difluorophenyl)-1H-imidazol-2-yl)piperidin-1-yl)-2-(methylthio)propan-1-one,
1-(2-(5-(2,5-difluorophenyl)-1H-imidazol-2-yl)piperidin-1-yl)-2-(methylthio)propan-1-one,
1-(2-(5-(2-fluoro-4-methoxyphenyl)-1H-imidazol-2-yl)piperidin-1-yl)-2-(methylthio)propan-1-one,
1-(2-(5-(2-fluoro-4-methylphenyl)-1H-imidazol-2-yl)piperidin-1-yl)-2-(methylthio)propan-1-one,
1-(2-(5-(2-methoxypyrimidin-5-yl)-1H-imidazol-2-yl)piperidin-1-yl)-2-(methylthio)propan-1-one,
1-(2-(5-(3-(methylamino)phenyl)-1H-imidazol-2-yl)piperidin-1-yl)-2-(methylthio)propan-1-one,
1-(2-(5-(3,4-difluorophenyl)-1H-imidazol-2-yl)piperidin-1-yl)-2-(methylthio)propan-1-one,
1-(2-(5-(3,4-difluorophenyl)-1H-imidazol-2-yl)piperidin-1-yl)-2-methylbut-3-en-1-one,
1-(2-(5-(3,4-dimethoxyphenyl)-1H-imidazol-2-yl)piperidin-1-yl)-2-(methylthio)propan-1-one,
1-(2-(5-(3,4-dimethylphenyl)-1H-imidazol-2-yl)piperidin-1-yl)-2-(methylthio)propan-1-one,
1-(2-(5-(3-fluoro-4-(methoxymethyl)phenyl)-1H-imidazol-2-yl)piperidin-1-yl)-2-(methylthio)propan-1-one,
1-(2-(5-(3-fluoro-4-methylphenyl)-1H-imidazol-2-yl)piperidin-1-yl)-2-(methylthio)propan-1-one,
1-(2-(5-(3-fluorophenyl)-1H-imidazol-2-yl)piperidin-1-yl)-2-(methylthio)propan-1-one,
1-(2-(5-(3-methoxyisothiazol-4-yl)-1H-imidazol-2-yl)piperidin-1-yl)-2-(methylthio)propan-1-one,
1-(2-(5-(3-methoxyphenyl)-1H-imidazol-2-yl)piperidin-1-yl)-2-(methylthio)propan-1-one,
1-(2-(5-(4-((methylamino)methyl)phenyl)-1H-imidazol-2-yl)piperidin-1-yl)-2-(methylthio)propan-1-one,
1-(2-(5-(4-(2-methoxyethyl)phenyl)-1H-imidazol-2-yl)piperidin-1-yl)-2-(methylthio)propan-1-one,
1-(2-(5-(4-(aminomethyl)phenyl)-1H-imidazol-2-yl)piperidin-1-yl)-2-(methylthio)propan-1-one,
1-(2-(5-(4-(dimethylamino)phenyl)-1H-imidazol-2-yl)piperidin-1-yl)-2-(methylthio)propan-1-one,
1-(2-(5-(4-(hydroxymethyl)phenyl)-1H-imidazol-2-yl)piperidin-1-yl)-2-(methylthio)propan-1-one,
1-(2-(5-(4-acetylphenyl)-1H-imidazol-2-yl)piperidin-1-yl)-2-(methylthio)propan-1-one,
1-(2-(5-(4-aminophenyl)-1H-imidazol-2-yl)piperidin-1-yl)-2-(methylthio)propan-1-one,
1-(2-(5-(4-chloro-3-fluorophenyl)-1H-imidazol-2-yl)piperidin-1-yl)-2-(methylthio)propan-1-one,
1-(2-(5-(4-chlorophenyl)-1H-imidazol-2-yl)piperidin-1-yl)-2-(methylthio)propan-1-one,
1-(2-(5-(4-ethylphenyl)-1H-imidazol-2-yl)piperidin-1-yl)-2-(methylthio)propan-1-one,
1-(2-(5-(4-fluorophenyl)-4H-1,2,4-triazol-3-yl)piperidin-1-yl)-2-(methylthio)propan-1-one,
1-(2-(5-(4-methoxy-2-methylphenyl)-1H-imidazol-2-yl)piperidin-1-yl)-2-(methylthio)propan-1-one,
1-(2-(5-(4-methoxyphenyl)-1H-imidazol-2-yl)piperidin-1-yl)-2-(methylthio)propan-1-one,
1-(2-(5-(4-methyl-6-(methylthio)pyridin-3-yl)-1H-imidazol-2-yl)piperidin-1-yl)-2-(methylthio)propan-1-one,
1-(2-(5-(5-(methoxymethyl)thiophen-3-yl)-1H-imidazol-2-yl)piperidin-1-yl)-2-(methylthio)propan-1-one,
1-(2-(5-(6-methoxypyridin-3-yl)-1H-imidazol-2-yl)piperidin-1-yl)-2-(methylthio)propan-1-one,
1-(2-(5-(benzo[d][1,3]dioxol-5-yl)-1H-imidazol-2-yl)piperidin-1-yl)-2-(methylthio)propan-1-one,
1-(2-(5-(p-tolyl)-1H-imidazol-2-yl)piperidin-1-yl)propan-1-one,
1-(2-(5-benzyl-1H-imidazol-2-yl)piperidin-1-yl)-2-(methylthio)propan-1-one,
1-(2-(5-bromo-1H-imidazol-2-yl)piperidin-1-yl)-2-(methylthio)propan-1-one,
2-(2-(1-(2-(methylthio)propanoyl)piperidin-2-yl)-1H-imidazol-5-yl)benzonitrile,
2-(2-(4-phenyl-1H-imidazol-2-yl)piperidine-1-carbonyl)cyclopropane-1-carbonitrile,
2-(5-methylfuran-2-yl)-1-(2-(4-phenyl-1H-imidazol-2-yl)piperidin-1-yl)propan-1-one,
2-(allylthio)-1-(2-(5-(p-tolyl)-1H-imidazol-2-yl)piperidin-1-yl)propan-1-one,
2-(dimethylamino)-1-(2-(4-phenyl-1H-imidazol-2-yl)piperidin-1-yl)butan-1-one,
2-(dimethylamino)-1-(2-(4-phenyl-1H-imidazol-2-yl)piperidin-1-yl)propan-1-one,
2-(ethylthio)-1-(2-(5-(p-tolyl)-1H-imidazol-2-yl)piperidin-1-yl)propan-1-one,
2-(furan-2-yl)-1-(2-(4-phenyl-1H-imidazol-2-yl)piperidin-1-yl)ethan-1-one,
2-(furan-2-yl)-1-(2-(4-phenyl-1H-imidazol-2-yl)piperidin-1-yl)propan-1-one,
2-(furan-3-yl)-1-(2-(4-phenyl-1H-imidazol-2-yl)piperidin-1-yl)ethan-1-one,
2-(furan-3-yl)-1-(2-(5-(p-tolyl)-1H-imidazol-2-yl)piperidin-1-yl)propan-1-one,
2-(methylamino)-N-(4-(2-(1-(2-(methylthio)propanoyl)piperidin-2-yl)-1H-imidazol-5-yl)phenyl)acetamide,
2-(methylsulfinyl)-1-(2-(5-(p-tolyl)-1H-imidazol-2-yl)piperidin-1-yl)propan-1-one,
2-(methylsulfonyl)-1-(2-(4-phenyl-1H-imidazol-2-yl)piperidin-1-yl)ethan-1-one,
2-(methylsulfonyl)-1-(2-(4-phenyl-1H-imidazol-2-yl)piperidin-1-yl)propan-1-one,
2-(methylthio)-1-(2-(2-(p-tolyl)-2H-tetrazol-5-yl)piperidin-1-yl)propan-1-one,
2-(methylthio)-1-(2-(3-phenyl-1,2,4-oxadiazol-5-yl)piperidin-1-yl)propan-1-one,
2-(methylthio)-1-(2-(4-(1-(trifluoromethyl)-1H-pyrazol-4-yl)-1H-imidazol-2-yl)piperidin-1-yl)propan-1-one,
2-(methylthio)-1-(2-(4-(4-(morpholinomethyl)phenyl)-1H-imidazol-2-yl)piperidin-1-yl)propan-1-one,
2-(methylthio)-1-(2-(4-(5-(trifluoromethyl)furan-2-yl)-1H-imidazol-2-yl)piperidin-1-yl)propan-1-one,
2-(methylthio)-1-(2-(4-(5-(trifluoromethyl)thiophen-2-yl)-1H-imidazol-2-yl)piperidin-1-yl)propan-1-one,
2-(methylthio)-1-(2-(4-(5-methylthiophen-3-yl)-1H-imidazol-2-yl)piperidin-1-yl)propan-1-one,
2-(methylthio)-1-(2-(4-(m-tolyl)-1H-imidazol-2-yl)piperidin-1-yl)propan-1-one,
2-(methylthio)-1-(2-(4-(o-tolyl)-1H-imidazol-2-yl)piperidin-1-yl)propan-1-one,
2-(methylthio)-1-(2-(4-(pyridin-3-yl)-1H-imidazol-2-yl)piperidin-1-yl)propan-1-one,
2-(methylthio)-1-(2-(4-(pyridin-4-yl)-1H-imidazol-2-yl)piperidin-1-yl)propan-1-one,
2-(methylthio)-1-(2-(4-(pyrimidin-5-yl)-1H-imidazol-2-yl)piperidin-1-yl)propan-1-one,
2-(methylthio)-1-(2-(4-(quinoxalin-6-yl)-1H-imidazol-2-yl)piperidin-1-yl)propan-1-one,
2-(methylthio)-1-(2-(4-(thiophen-3-yl)-1H-imidazol-2-yl)piperidin-1-yl)propan-1-one,
2-(methylthio)-1-(2-(4-phenyl-1H-imidazol-2-yl)piperidin-1-yl)ethan-1-one,
2-(methylthio)-1-(2-(5-(2-(trifluoromethyl)phenyl)-1H-imidazol-2-yl)piperidin-1-yl)propan-1-one,
2-(methylthio)-1-(2-(5-(2,3,4-trifluorophenyl)-1H-imidazol-2-yl)piperidin-1-yl)propan-1-one,
2-(methylthio)-1-(2-(5-(2,4,5-trifluorophenyl)-1H-imidazol-2-yl)piperidin-1-yl)propan-1-one,
2-(methylthio)-1-(2-(5-(3-(trifluoromethyl)phenyl)-1H-imidazol-2-yl)piperidin-1-yl)propan-1-one,
2-(methylthio)-1-(2-(5-(4-(trifluoromethoxy)phenyl)-1H-imidazol-2-yl)piperidin-1-yl)propan-1-one,
2-(methylthio)-1-(2-(5-(4-(trifluoromethyl)phenyl)-1H-imidazol-2-yl)piperidin-1-yl)propan-1-one,
2-(methylthio)-1-(2-(5-(4-propylphenyl)-1H-imidazol-2-yl)piperidin-1-yl)propan-1-one,
2-(methylthio)-1-(2-(5-(4-vinylphenyl)-1H-imidazol-2-yl)piperidin-1-yl)propan-1-one,
2-(methylthio)-1-(2-(5-(5-methylthiophen-2-yl)-1H-imidazol-2-yl)piperidin-1-yl)propan-1-one,
2-(methylthio)-1-(2-(5-(p-tolyl)-1,3,4-oxadiazol-2-yl)piperidin-1-yl)propan-1-one,
2-(methylthio)-1-(2-(5-(p-tolyl)-1H-imidazol-2-yl)azepan-1-yl)propan-1-one,
2-(methylthio)-1-(2-(5-(p-tolyl)-1H-imidazol-2-yl)piperidin-1-yl)propan-1-one,
2-(methylthio)-1-(2-(5-(p-tolyl)-4H-1,2,4-triazol-3-yl)piperidin-1-yl)propan-1-one,
2-(methylthio)-1-(2-(5-(p-tolyl)isoxazol-3-yl)piperidin-1-yl)propan-1-one,
2-(methylthio)-1-(2-(5-(p-tolyl)oxazol-2-yl)piperidin-1-yl)propan-1-one,
2-(methylthio)-1-(2-(5-(p-tolyl)thiazol-2-yl)piperidin-1-yl)propan-1-one,
2-(methylthio)-1-(2-(5-phenyl-1H-imidazol-2-yl)piperidin-1-yl)propan-1-one,
2-(methylthio)-1-(2-(5-phenyl-1H-imidazol-2-yl)pyrrolidin-1-yl)propan-1-one,
2-(methylthio)-1-(2-(5-phenyl-4H-1,2,4-triazol-3-yl)piperidin-1-yl)propan-1-one,
2-(methylthio)-1-(2-(5-phenyloxazol-2-yl)piperidin-1-yl)propan-1-one,
2-(methylthio)-1-(4-(5-(p-tolyl)-1H-imidazol-2-yl)thiazolidin-3-yl)propan-1-one,
2,2,3-trimethyl-1-(2-(5-(p-tolyl)-1H-imidazol-2-yl)piperidin-1-yl)but-3-en-1-one,
2,2,3-trimethyl-1-(2-(5-(p-tolyl)-1H-imidazol-2-yl)piperidin-1-yl)butan-1-one,
2,2-dimethyl-1-(2-(5-(p-tolyl)-1H-imidazol-2-yl)piperidin-1-yl)but-3-en-1-one,
2,2-dimethyl-1-(2-(5-(p-tolyl)-1H-imidazol-2-yl)piperidin-1-yl)butan-1-one,
2,3-dimethoxy-1-(2-(4-phenyl-1H-imidazol-2-yl)piperidin-1-yl)propan-1-one,
2,3-dimethyl-1-(2-(4-phenyl-1H-imidazol-2-yl)piperidin-1-yl)butan-1-one,
2,5-difluoro-4-(2-(1-(2-(methylthio)propanoyl)piperidin-2-yl)-1H-imidazol-4-yl)benzonitrile,
2-amino-1-(2-(5-(p-tolyl)-1H-imidazol-2-yl)piperidin-1-yl)propan-1-one,
2-cyclobutyl-1-(2-(4-phenyl-1H-imidazol-2-yl)piperidin-1-yl)propan-1-one,
2-cyclopentyl-2-ethoxy-1-(2-(4-phenyl-1H-imidazol-2-yl)piperidin-1-yl)ethan-1-one,
2-cyclopropyl-1-(2-(4-phenyl-1H-imidazol-2-yl)piperidin-1-yl)ethan-1-one,
2-cyclopropyl-1-(2-(4-phenyl-1H-imidazol-2-yl)piperidin-1-yl)propan-1-one,
2-cyclopropyl-2-methoxy-1-(2-(4-phenyl-1H-imidazol-2-yl)piperidin-1-yl)ethan-1-one,
2-ethoxy-1-(2-(5-(3-fluorophenyl)-1H-imidazol-2-yl)piperidin-1-yl)propan-1-one,
2-ethyl-1-(2-(4-phenyl-1H-imidazol-2-yl)piperidin-1-yl)but-2-en-1-one,
2-ethyl-1-(2-(4-phenyl-1H-imidazol-2-yl)piperidin-1-yl)butan-1-one,
2-fluoro-4-(2-(1-(2-(methylthio)propanoyl)piperidin-2-yl)-1H-imidazol-5-yl)benzonitrile,
2-mercapto-1-(2-(5-(p-tolyl)-1H-imidazol-2-yl)piperidin-1-yl)propan-1-one,
2-methoxy-1-(2-(4-phenyl-1H-imidazol-2-yl)piperidin-1-yl)butan-1-one,
2-methoxy-1-(2-(4-phenyl-1H-imidazol-2-yl)piperidin-1-yl)propan-1-one,
2-methoxy-1-(2-(5-(p-tolyl)-1H-imidazol-2-yl)piperidin-1-yl)propan-1-one,
2-methyl-1-(2-(4-phenyl-1H-imidazol-2-yl)piperidin-1-yl)butan-1-one,
2-methyl-1-(2-(4-phenyl-1H-imidazol-2-yl)piperidin-1-yl)propan-1-one,
2-methyl-1-(2-(5-(p-tolyl)-1H-imidazol-2-yl)piperidin-1-yl)but-3-en-1-one,
2-methyl-1-(2-(5-(p-tolyl)-1H-imidazol-2-yl)piperidin-1-yl)butan-1-one,
2-methyl-1-(2-(5-(p-tolyl)-1H-imidazol-2-yl)piperidin-1-yl)butane-1,3-dione,
2-methyl-1-(2-(5-(p-tolyl)-1H-imidazol-2-yl)piperidin-1-yl)pentan-1-one,
2-methyl-1-(2-(5-(p-tolyl)-1H-imidazol-2-yl)piperidin-1-yl)prop-2-en-1-one,
2-methyl-1-(2-(5-(p-tolyl)-1H-imidazol-2-yl)piperidin-1-yl)propan-1-one,
2-methyl-2-(methylthio)-1-(2-(5-(p-tolyl)-1H-imidazol-2-yl)piperidin-1-yl)propan-1-one,
2-methyl-3-(methylthio)-1-(2-(4-phenyl-1H-imidazol-2-yl)piperidin-1-yl)propan-1-one,
2-methylene-1-(2-(5-(p-tolyl)-1H-imidazol-2-yl)piperidin-1-yl)butan-1-one,
3-(2-(1-(2-(methylthio)propanoyl)piperidin-2-yl)-1H-imidazol-4-yl)benzonitrile,
3,3,3-trifluoro-2-methyl-1-(2-(4-phenyl-1H-imidazol-2-yl)piperidin-1-yl)propan-1-one,
3,3,4,4,4-pentafluoro-1-(2-(4-phenyl-1H-imidazol-2-yl)piperidin-1-yl)butan-1-one,
3,3-difluoro-1-(2-(4-phenyl-1H-imidazol-2-yl)piperidin-1-yl)butan-1-one,
3-fluoro-4-(2-(1-(2-(methylthio)propanoyl)piperidin-2-yl)-1H-imidazol-5-yl)benzonitrile,
3-methyl-1-(2-(4-phenyl-1H-imidazol-2-yl)piperidin-1-yl)but-2-en-1-one,
3-methyl-1-(2-(4-phenyl-1H-imidazol-2-yl)piperidin-1-yl)butan-1-one,
4-(2-(1-(2-(methylthio)propanoyl)piperidin-2-yl)-1H-imidazol-4-yl)benzonitrile,
4-(2-(1-(2-(methylthio)propanoyl)piperidin-2-yl)-1H-imidazol-5-yl)benzaldehyde,
4,4,4trifluoro-2-methyl-1-(2-(4-phenyl-1H-imidazol-2-yl)piperidin-1-yl)butan-1-one,
bicyclo[1.1.1 ]pentan-1-yl(2-(4-phenyl-1H-imidazol-2-yl)piperidin-1-yl)methanone,
cyclobutyl(2-(5-(3-fluorophenyl)-1H-imidazol-2-yl)piperidin-1-yl)methanone,
cyclopentyl(2-(4-phenyl-1H-imidazol-2-yl)piperidin-1-yl)methanone,
cyclopropyl(2-(4-phenyl-1H-imidazol-2-yl)piperidin-1-yl)methanone,
methyl-4-(2-(1-(2-(methylthio)propanoyl)piperidin-2-yl)-1H-imidazol-5-yl)benzoate,
N-(2-hydroxyethyl)-4-(2-(1-(2-(methylthio)propanoyl)piperidin-2-yl)-1H-imidazol-4-yl)benzenesulfonamide,
2-(methylthio)-1-(2-(5-(3-nitrophenyl)-1H-imidazol-2-yl)piperidin-1-yl)propan-1-one,
N-(4-(2-(1-(2-(methylthio)propanoyl)piperidin-2-yl)-1H-imidazol-4-yl)phenyl)methane-sulfonamide,
2-(methylthio)-1-(2-(5-(4-nitrophenyl)-1H-imidazol-2-yl)piperidin-1-yl)propan-1-one,
1-(sec-butylsulfinyl)-2-(5-(p-tolyl)-1H-imidazol-2-yl)piperidine,
1-(sec-butylsulfonyl)-2-(5-(p-tolyl)-1H-imidazol-2-yl)piperidine,
1-(isopropylsulfinyl)-2-(5-(p-tolyl)-1H-imidazol-2-yl)piperidine,
1-(isopropylsulfonyl)-2-(5-(p-tolyl)-1H-imidazol-2-yl)piperidine,
1-((1-(methylthio)ethyl)sulfinyl)-2-(5-(p-tolyl)-1H-imidazol-2-yl)piperidine,
1-((1-(methylthio)ethyl)sulfonyl)-2-(5-(p-tolyl)-1H-imidazol-2-yl)piperidine,
1-((2-(methylthio)propan-2-yl)sulfinyl)-2-(5-(p-tolyl)-1H-imidazol-2-yl)piperidine,
1-((2-(methylthio)propan-2-yl)sulfonyl)-2-(5-(p-tolyl)-1H-imidazol-2-yl)piperidine,
1-(but-3-en-2-ylsulfinyl)-2-(5-(p-tolyl)-1H-imidazol-2-yl)piperidine,
1-(but-3-en-2-ylsulfonyl)-2-(5-(p-tolyl)-1H-imidazol-2-yl)piperidine,
1-(pent-3-en-2-ylsulfinyl)-2-(5-(p-tolyl)-1H-imidazol-2-yl)piperidine,
1-(pent-3-en-2-ylsulfonyl)-2-(5-(p-tolyl)-1H-imidazol-2-yl)piperidine,
1-((2-methylbut-3-en-2-yl)sulfinyl)-2-(5-(p-tolyl)-1H-imidazol-2-yl)piperidine,
1-((2-methylbut-3-en-2-yl)sulfonyl)-2-(5-(p-tolyl)-1H-imidazol-2-yl)piperidine,
1-(2-(5-(4-(hydroxymethyl)-phenyl)-1H-imidazol-2-yl)piperidin-1-yl)-2-methylbutan-1-one,
2-(methylsulfonyl)-1-(2-(5-(p-tolyl)-1H-imidazol-2-yl)piperidin-1-yl)propan-1-one,
1-(4-hydroxy-2-(5-(p-tolyl)-1H-imidazol-2-yl)piperidin-1-yl)-2-(methylthio)propan-1-one,
1-(5-hydroxy-2-(5-(p-tolyl)-1H-imidazol-2-yl)piperidin-1-yl)-2-(methylthio)propan-1-one,
1-(4-hydroxy-2-(5-(p-tolyl)-1H-imidazol-2-yl)piperidin-1-yl)-2-methylbutan-1-one,
1-(5-hydroxy-2-(5-(p-tolyl)-1H-imidazol-2-yl)piperidin-1-yl)-2-methylbutan-1-one,
1-(2-(5-(4-(hydroxymethyl)phenyl)-1H-imidazol-2-yl)piperidin-1-yl)-2-(methylsulfonyl)propan-1-one,
1-(4-hydroxy-2-(5-(p-tolyl)-1H-imidazol-2-yl)piperidin-1-yl)-2-(methylsulfonyl)propan-1-one,
1-(5-hydroxy-2-(5-(p-tolyl)-1H-imidazol-2-yl)piperidin-1-yl)-2-(methylsulfonyl)propan-1-one,
1-(2-hydroxy-6-(5-(p-tolyl)-1H-imidazol-2-yl)piperidin-1-yl)-2-(methylthio)propan-1-one,
1-(2-hydroxy-6-(5-(p-tolyl)-1H-imidazol-2-yl)piperidin-1-yl)-2-methylbutan-1-one,
1-(2-hydroxy-6-(5-(p-tolyl)-1H-imidazol-2-yl)piperidin-1-yl)-2-(methylsulfonyl)propan-1-one,
1-(3-hydroxy-2-(5-(p-tolyl)-1H-imidazol-2-yl)piperidin-1-yl)-2-(methylthio)propan-1-one,
1-(3-hydroxy-2-(5-(p-tolyl)-1H-imidazol-2-yl)piperidin-1-yl)-2-methylbutan-1-one,
1-(3-hydroxy-2-(5-(p-tolyl)-1H-imidazol-2-yl)piperidin-1-yl)-2-(methylsulfonyl)propan-1-one,
1-(2-(5-(4-(hydroxymethyl)phenyl)-1H-imidazol-2-yl)piperidin-1-yl)-2-(methylsulfinyl)-propan-1-one,
1-(4-hydroxy-2-(5-(p-tolyl)-1H-imidazol-2-yl)piperidin-1-yl)-2-(methylsulfinyl)propan-1-one,
1-(5-hydroxy-2-(5-(p-tolyl)-1H-imidazol-2-yl)piperidin-1-yl)-2-(methylsulfinyl)propan-1-one,
1-(2-hydroxy-6-(5-(p-tolyl)-1H-imidazol-2-yl)piperidin-1-yl)-2-(methylsulfinyl)propan-1-one,
1-(3-hydroxy-2-(5-(p-tolyl)-1H-imidazol-2-yl)piperidin-1-yl)-2-(methylsulfinyl)propan-1-one,
1-(2-hydroxy-6-(5-(4-hydroxymethyl)phenyl)-1H-imidazol-2-yl)piperidin-1-yl)-2-(methylthio)propan-1-one,
1-(5-hydroxy-2-(5-(4-(hydroxymethyl)phenyl)-1H-imidazol-2-yl)piperidin-1-yl)-2-(methylthio)propan-1-one,
1-(4-hydroxy-2-(5-(4-(hydroxymethyl)phenyl)-1H-imidazol-2-yl)piperidin-1-yl)-2-(methylthio)propan-1-one,
1-(3-hydroxy-2-(5-(4-(hydroxymethyl)-phenyl)-1H-imidazol-2-yl)piperidin-1-yl)-2-(methylthio)propan-1-one,
1-(2-hydroxy-6-(5-(4-(hydroxymethyl)phenyl)-1H-imidazol-2-yl)piperidin-1-yl)-2-methylbutan-1-one,
1-(5-hydroxy-2-(5-(4-hydroxymethyl)phenyl)-1H-imidazol-2-yl)piperidin-1-yl)-2-methylbutan-1-one,
1-(4-hydroxy-2-(5-(4-(hydroxymethyl)phenyl)-1H-imidazol-2-yl)piperidin-1-yl)-2-methylbutan-1-one,
1-(3-hydroxy-2-(5-(4-(hydroxymethyl)phenyl)-1H-imidazol-2-yl)piperidin-1-yl)-2-methylbutan-1-one,
4-(2-(1-(2-methylbutanoyl)piperidin-2-yl)-1H-imidazol-5-yl)benzaldehyde,
4-(2-(1-(2-(methylthio)propanoyl)piperidin-2-yl)-1H-imidazol-5-yl)benzoic acid,
4-(2-(1-(2-methylbutanoyl)piperidin-2-yl)-1H-imidazol-5-yl)benzoic acid,
(2,2-dimethylcyclopropyl)(2-(5-(p-tolyl)-1H-imidazol-2-yl)piperidin-1-yl)methanone,
1-(2-(5-bromo-1H-imidazol-2-yl)piperidin-1-yl)-2-methylbutan-1-one,
1-(2-(5-(4-chlorophenyl)-1H-imidazol-2-yl)piperidin-1-yl)-2-methylbutan-1-one,
2-(4-(2-(1-(2-(methylthio)propanoyl)piperidin-2-yl)-1H-imidazol-5-yl)phenyl)acetonitrile,
2-(4-(2-(1-(2-methylbutanoyl)piperidin-2-yl)-1H-imidazol-5-yl)phenyl)acetonitrile,
2-(4-(2-(1-(2-methylbutanoyl)piperidin-2-yl)-1H-imidazol-5-yl)phenyl)acetonitrile,
2,3-dimethyl-1-(2-(5-(p-tolyl)-1H-imidazol-2-yl)piperidin-1-yl)butan-1-one,
2-methyl-1-(2-(5-(o-tolyl)-1H-imidazol-2-yl)piperidin-1-yl)butan-1-one,
2-methyl-1-(2-(5-(m-tolyl)-1H-imidazol-2-yl)piperidin-1-yl)butan-1-one,
1-(2-(5-(4-(hydroxymethyl)phenyl)-1H-imidazol-2-yl)piperidin-1-yl)-2-methylbutan-1-one,
1-(isopropylsulfonyl)-2-(5-(p-tolyl)-1H-imidazol-2-yl)piperidine,
1-(sec-butylsulfonyl)-2-(5-(p-tolyl)-1H-imidazol-2-yl)piperidine,
2-methyl-1-(2-(5-(5-methylthiophen-2-yl)-1H-imidazol-2-yl)piperidin-1-yl)butan-1-one,
2-(methylthio)-1-(2-(5-(p-tolyl)-1H-imidazol-2-yl)piperazin-1-yl)propan-1-one,
2-(methylthio)-1-(2-(4-(p-tolyl)oxazol-2-yl)piperidin-1-yl)propan-1-one,
2-methyl-1-(2-(4-(p-tolyl)oxazol-2-yl)piperidin-1-yl)butan-1-one,
and mixtures thereof.

6. The composition of claim 1, wherein said physiological cooling agent forming group (b1) is are selected from the group consisting of:
Menthol,
Menthol glyceryl acetal,
Menthol glyceryl ketal,
Menthol menthyl ether,
Menthone glyceryl acetal,
Menthone glyceryl ketal,
Menthoxy-1,2-propandiol,
Menthoxy-2-methyl-1,2-propanediol,
Menthyl acetate,
Menthyl ethylene glycol carbonate,
Menthyl formiate,
Menthyl glutamate,
Menthyl glycerol carbonate,
Menthyl hydroxy isobutyrate,
Menthyl isobutyrate,
Menthyl lactate,
Menthyl malonate,
Menthyl methyl ether,
Menthyl N-ethyl oxamate,
Menthyl propylene glycol carbonate,
Menthyl pyroglutamate,
Menthyl-(2-methoxy)acetate,
Menthyl-(2-methoxyethoxy)acetate,
Menthyl succinate,
O-Menthyl succinic acid ester amide,
O-Menthyl succinic acid ester-NN-(dimethyl)amide,
Menthane carboxylic acid-N-(4-cyanophenyl)amide,
Menthane carboxylic acid-N-(4-cyanomethylphenyl)amide,
Menthane carboxylic acid-N-ethylamide (WS-3),
(WS-4),
Nα-(menthane carbonyl) glycine ethylester (WS-5),
(1R,2S,5R)—N-(4-Methoxyphenyl)-5-methyl-2-(1-isopropyl)cyclohexane-carboxamide (WS-12),
(WS-14),
2,3-dimethyl-2-(2-propyl)-butyric acid-N-methylamide (WS23),
Isopulegol acetate,
p-Menthane-3,8-diol,
Cubebol,
3-Methyl-2(1-pyrrolidinyl)-2-cyclopentene-1-one),
Tetrahydropyrimidine-2-one,
N-(2-(pyridin-2-yl)ethyl)-3-p-menthanecarboxamide,
[(1R,2S,5R)-2-isopropyl-5-methyl-cyclohexyl] 2-(ethylamino)-2-oxoacetate,
and mixtures thereof.

7. The composition of claim 1, wherein said physiological warming agent forming group (b2) is are selected from the group consisting of:
vanillyl derivatives;
capsaicin;
allyl isothiocyanate;
gingerol;
4-(l-menthoxymethyl)-2-phenyl-I;3-dioxolan;
4-(l-menthoxymethyl)-2-(3′;4′-dihydroxyphenyl)-I;3-dioxolan;
4-(l-menthoxymethyl)-2-(2′-hydroxy-3′-methoxyphenyl)-3-dioxolan;
4-(l-menthoxymethyl)-2-(4′-methoxyphenyl)-3-dioxolan;
4-(l-menthoxymethyl)-2-(3′;4′methylenedioxyphenyl)-3-dioxolan;
4-(l-menthoxymethyl)-2-(3′-methoxy-4′-hydroxyphenyl)-3-dioxolan;
red pepper oil;
red pepper oleoresin;
ginger oleoresin;
nonylic acid vanillyl amide;
jambu oleoresin;
Zanthoxylum pieritum extract;
sanshool I;
sanshool II;
sanshoamide;
black pepper extract;
chavicine;
piperine;
spilanthol;
and mixtures thereof.

8. The composition of claim 1, wherein said flavoring compound is selected from the group consisting of:
12-methyltridecanal;
acetaldehyde;
acetanisole;
acethylthiazole-2;
acethylthiazoline-2;
acetic acid;
acetyl methylcarbinol;
acetyl pyrazine-2;
acetyl pyridine-2;
allyl capronate;
allyl isothiocyanate;
amyl cinnamaldehyde-alpha;
anethole;
anisaldehyde-para;
anisic alcohol;
benzaldehyde;
benzyl acetate;
borneol-l;
butyl acetate;
butylidene phthalide-3;
butyric acid;
camphor;
caproic acid;
carvacrol;
carvomenthone;
carvon-d;
carvon-l;
carvylacetate-cis;
caryophyllene;
cineole-1;4;
cineole-1;8;
cinnamalcohol;
cinnamaldehyde;
cinnamyl acetate;
citral;
citronellal;
citronellol;
citronellyl acetate;
cresol;
cuminaldehyde;
cyclopentadecanolide;
damascenone-alpha;
damascenone-beta;
damascone-alpha;
damascoe-beta;
decadienal-2;4;
decadienal-2;4;
decalactone delta;
decalacton gamma;
decanal;
dehydromenthofurolactone;
diallyl disulfide;
diethylpyrazine-2;3;
dihydroanethol;
dihydrocarvone;
dihydrocoumarin;
dihydroionone-beta;
dihydromenthofurolactone;
dihydronootkatone;
dimethyl lanthranilate;
dimethyl isopropyldithiazine;
dimethyl pyrazine;
dimethyl sulfide;
diphenyloxide;
divanilline;
d-menthol;
d-menthone;
d-menthyl acetate;
dodecalactone-delta;
dodecalactone-gamma;
epithiomenthan-1;8;
ethyl acetate;
ethyl butyrate;
ethyl capronate;
ethyl caprylate;
ethyl cinnamate;
ethyl isobutyrate;
ethyl isovaleria-nate;
ethyl lactate;
ethyl maltol;
ethyl methyl butyrate-2;
ethyl methyl thiopropionate;
ethyl phenol-4;
ethyl vanillin;
eugenol;
fenchol;
filbertone;
frambinone methyl ether;
frambinone;
furaneol;
furfurylthiol;
geraniol;
geranyl acetate;
geranyl isobutyrate;
guaiacol;
heliotropin;
heptalactone-gamma;
heptanone-2;
heptenal-Z-4;
hexalactone-gamma;
hexanal;
hexanol;
hexenol-E-2;
hexenol-Z-3;
hexenyl acetate-E-2;
hexenyl acetate-Z-3;
hexenyl capronate-Z-3;
hexyl acetate;
hexyl cinnamaldehyde-alpha;
hotrienol;
indole;
ionon-beta;
ionone-alpha;
isoamyl acetate;
isoamyl butyrate;
isoamyl isovaerianate;
isobutyl acetate;
isobutyl thiazole;
isobutylmethoxypyrazine;
isobutyraldehyde;
isoeugenol;
Isomenthon;
isopropylmethoxypyrazine;
isopropylmethylpyrazine-2;4;
isopulegol;
isova-leraldehyde;
jasmine lactone;
jasmon cis;
ketoisophorone;
limonene-d;
linalool oxide;
linalool-d;
linalool-l;
linalyl acetate;
l-menthol;
l-menthone;
l-menthyl acetate;
maltol;
massoilactone;
melonal;
menthadienyl acetate;
menthofurolactone;
menththiol-1;8;
methional;
methoxymethylpyrazine-2;3;
methyl anthranilate;
methyl butyl acetate-2;
methyl butyrate;
methyl cinnamate;
methyl dihydrojasmonate;
methyl furanthiol-2;3;
methyl jasmonate;
methyl salicylate;
methyl tetrahydrofuranthiol-2;3;
methyl thiobutyrate;
methyl thiohexyl acetate-1;3;
methylcyclopen-tenolone;
methylthiohexanol-1;3;
myrtenal;
naringin;
neo-menthol;
neral;
nerol;
nerylacetate;
nonadienal-2;4;
nonadienal-2;6;
nonalactone delta;
nonalactone gamma;
nonenal-E-2;
nonenal-Z-6;
nonenol-Z-6;
nootkatone;
octalactone gamma;
octalactone delta;
octanal;
octenol-1;3;
pellitorin;
pentenone-1;3;
pentyl acetate;
phenyl ethyl acetate;
phenyl ethyl alcohol;
phenyl acetaldehyde;
pinene-alpha;
pinene-beta;
piperitanate;
piperitone;
prenyl thioacetate;
prenyl thiol;
reubenamine;
rose oxide;
rubescenamine;
sabinene hydrate;
scatole;
sotolone;
styrolyl acetate;
terpinenol-4;
terpineol;
thiohexanol-1;3;
thiohexyl acetate-1;3;
thiomenthanone-8;3;
thiopentanone-4;4;2;
thymol;
tocopherol;
tridecatrienal;
trimethyl pyrazine;
undecadienal-2;4;
undecalactone-gamma and delta;
undecatriene;
vanillin;
vinyl guaiacol;
whisky lactone;
and mixtures thereof.

9. The composition of claim 1, wherein said fragrance forming group (d) is selected from the group consisting of:
[(3,7-dimethyl-6-octenyl)-xy]-acetaldehyde,
1-(p-menthene-6(2)-yl)-1-propanone,
1,2,3,4,5,6,7,8-octahydro-8,8-dimethyl-2-naphthaldehyde,
10-undecen-1-al,
10-undecen-1-ol,
1-decanal,
1-dodecanal,
1-methyl-3-(4-methylpentyl)-3-cyclohexene-carboxyaldehyde,
1-methyl-4-(4-methyl-3-pentenyl)-3-cyclohexene-1-carboxaldehyde,
1-nonanal,
1-octanal,
1-octen-3-ol,
1-p-menthene-q-carboxaldehyde,
1-undecanal,
2-(2-(4-Methyl-3-cyclohexen-1-yl)propyl)-cyclopentanone,
2,4,4,7-tetramethyl-oct-6-en-3-one,
2,4,6-trimethyl-3-cyclohexene-1-carboxalde-hyde,
2,4-dimethyl-3-cyclohexen-1-carboxaldehyde 4-methoxybenzaldehyde,
2,4-dimethyl-3-cyclohexen-1-carboxyaldehyde,
2,6,10-trimethyl-5,9-undecadienal,
2,6,10-trimethyl-9-undecenal, 3-dodecen-1-al,
2,6-dimethyl-5-heptenal;
2,6-dimethylhept-5-en-1-al,
2,6-dimethylheptan-2-ol,
2,6-nonadienal,
2-acetyl-3,3-di-methyl-norbornan,
2-methyl decanal,
2-methyl octanal,
2-methyl undecanal,
2-methyl-3-(4-tertbutyl)propanal,
2-methyl-3-(isopropyl-phenyl)propanal,
2-methyl-3-(para-methoxy-phenylpropanal),
2-methyl-3-phenyl-2-propen-1-al,
2-methyl-4-(2,6,6-trimethyl-1-cyclohexen-1-yl)-2-butenal,
2-methylbutanol,
2-methylpentanol,
2-phenoxyethanol,
2-phenylpropan-1-al,
2-phenylpropanol,
2-tert-butycyclohexanol,
3-(3,4-methylendioxy-phenyl)-2-methylpropan-1-al,
3-(3-isopropylphenyl)-butan-1-al,
3-(4-ethylphenyl)-2,2-dimethylpropan-1-al,
3-(4-ethylphenyl)-2,2-dimethylpropanal,
3-(4-isopropylphenyl)-2-methylpropan-1-al,
3-(4-methoxyphenyl)-2-methylpropanal,
3-(4-tert-butylphenyl)-2-methyl-propanal,
3-(4-tert-butylphenyl)-propanal,
3,4-dimethoxybenzaldehyde,
3,4-methylendioxybenzaldehyde,
3,5,5-trimethylcyclohexanol,
3,5,5-trimethylhexanal,
3,5,6-trimethyl-3-cyclohexenecarboxaldehyde,
3,7-dimethyl-2-methylene-6-octenal,
3,7-dimethyl-6-octen-1-al,
3,7-dimethyloctan-1-al,
3-ethoxy-4-hydroxybenzaldehyde,
3-hexanol,
3-methoxy-4-hydroxy-benzaldehyde,
3-methyl-5-phenyl-1-pentanal,
3-methyl-5-phenylpentanol,
3-octanol,
3-phenyl-2-hexylprop-2-enal,
3-phenyl-2-pentylprop-2-en-1-al,
3-phenyl-2-propenal,
3-phenylprop-2-en-1-al,
3-phenylpropanol,
3-propyl-bicyclo[2.2.1]-hept-5-ene-2-carbaldehyde,
4-(1-ethoxyvinyl)-3,3,5,5-tetramethyl cyclohexanone,
4-(3)(4-methyl-3-pentenyl)-3-cyclohexen-carboxaldehyde,
4-(4-hydroxy-3-methoxyphenyl)-2-butanone,
4-(4-hydroxy-4-methylpentyl)-3-cylohexene-1-carboxal-dehyde,
4-(tricyclo[5.2.1.0(2,6)]-decylidene-8)-butanal,
4-damascol,
4-heptenol,
4-hydroxy acetophenone;
4-hydroxy-3-methoxybenzaldehyde,
4-isopropyl benzaldehyde,
4-isopropyl cyclohexanol,
4-methoxy benzaldehyde,
4-methylphenyl acetaldehyde,
4-tert-butyl cyclohexanol,
5,9-dimethyl-4,8-decadienal,
5-methoxyhexahydro-4,7-methanoindan-1-carboxyaldehyde,
5-methoxyhexahydro-4,7-methanoindan-2-carboxyaldehyde,
6,10-dimethyl-3-oxa-5,9-undecadien-1-al,
6,6-dimethyl-2-norpinene-2-propionaldehyde,
6,7-dihydro-1,1,2,3,3-pentamethyl-4(5H)-indanone,
6,8-dimethyl-2-nonanol,6-nonen-1-ol,
6-isopropyl-deca-hydro-2-naphtone,
6-methoxyhexahydro-4,7-methanoindan-1-carboxyaldehyde,
6-methoxyhexahydro-4,7-methanoindan-2-carboxyaldehyde,
7-hydroxy-3,7-dimethyl octanal,
7-methoxy-3,7-dimethyloctan-1-al,
9-decen-1-ol,
9-decenal,
acetophenone,
adoxal,
allyl cyclohexylpropionate,
alphalonen,
ambroxan,
amylsalicylat,
anethol,
anisaldehyde,
benzaldehyde,
benzophenone,
benzyl acetate,
benzyl acetone,
benzyl alcohol,
benzyl salicylate,
benzylethyl ether,
bourgeonal,
buccoxime,
butyl lactate,
butyl salicylate,
campher,
canthoxal,
carvone,
cassione,
celery cetone,
chlorhydral,
cinnamalcohol,
cis-/trans-3,7-dimethyl-2,6-octadien-1-al,
cis-jasmon,
citral or its mixtures,
citronellal,
citronellol,
citronellyl oxyacetaldehyde,
cumal,
cyclamenaldehyde,
cyclohexyl salicylate,
cymal,
damarose,
damascenone,
decanol,
decyl aldehyde,
delphone,
dihydrojasmone,
dihydromyrcenol,
dihydrozimtaldehyde,
dihydro-p-lonone,
dimethyl benzylcarbinol,
dimethyl benzylcarbinylacetate,
dimethyl heptanol,
dimethyl octanol,
dimethyl octenone,
dulcinyl,
ethyl salicylate,
ethyl vanilin,
ethylmethylphenyl glycinate,
eugenol,
fenchone,
fleuramone,
floralozon,
floramat,
florhydral,
gelsone,
geraniol,
hedione,
helional,
heliotropin,
heptanol,
hexahydro-4,7-methanoindan-1-carboxaldehyde,
hexahydro-8,8-dimethyl-2-naphthaldehyde,
hexalone,
hexyl salicylat,
hydroxycitronellal,
isoborneol,
isodamascone,
isoeugenol,
isojasmone,
isopulegol,
jasmacyclatat,
koavon,
laurinaldehyde,
ligustral,
lilial citral,
lilial,
limonen,
linalool,
linalyl acetate,
livescon,
lyral,
m-cymene-7-carboxaldehyde,
melonal,
melusat,
menthol,
menthone,
methyl acetophenone,
methyl cedrenyl ketone,
methyl cedrylon,
methyl cedrylone,
methyl cyclocitrone,
methyl dihydrojasmonate,
methyl heptenone,
methyl lavender ketone,
methyl-dihydrojasmonate,
methylnonyl acetaldehyde,
methyl-p-naphthyl ketone,
moschus indanone,
muscone,
myrtenol,
n-dodecanal,
neobutenone,
nerol,
n-hexanol,
nonanol,
n-undecanal,
octahydro-4,7-methano-1H-indenecarboxaldehyde;
octanal,
octanol,
o-methoxyzimtaldehyde,
orivone,
p-ethyl-α,α-dimethylhydrozimtaldehyde,
phenol,
phenoxyacetaldehyde,
phenoxyisobutyrate,
phenyl acetaldehyde,
phenyl salicylat,
phenylethyl acetate,
phenylethyl alcohol,
phenylpropyl alcohol,
p-hydroxy phenylbutanone,
pinen,
plicatone,
p-menthan-7-ol,
p-methoxy acetophenone,
p-methyl phenoxy acetaldehyde,
p-tert.-bucinal,
p-tert.-butyl cyclohexanone,
p-tert.-butyl cyclohexylacetate,
p-tolyl acetaldehyde;
styrallyl propionate,
tetrahydrogeraniol,
tetrahydrolinalool,
tetrameran,
thymol,
tonalid/moschus plus,
trans-2-cis-6-nonadienol,
trans-2-nonen-1-ol,
trans-2-octenol,
trans-4-decenal,
triplal,
undecanal,
undecanol,
undecylenraldehyde,
vanillin,
veloutone,
verdone,
α.-methyl-3,4-(methylenedioxy)-hydrocinnamaldehyde,
α-damascone,
α-methyl benzylalcohol,
α-methyl-4-(l-methylethyl)benzene-acetaldehyde,
α-methylphenylacetaldehyde,
αn-Amylzimtaldehyde,
α-n-hexyl-cinnamaldehyde,
α-terpineol,
β-damascone,
β-ionon,
β-terpineol,
γ-methylionone,
δ-damascone,
and mixtures thereof.

10. A cosmetic preparation comprising the composition of claim 1.

11. The preparation of claim 9, which is skin care, personal care, hair care, sun care or oral care preparation.

12. The preparation of claim 9, comprising the composition in amounts of from about 0.01 to 1 wt.-percent—calculated on the preparation.

13. A foodstuff preparation comprising the composition of claim 1.

14. The preparation of claim 12, comprising the composition in amounts of from about 0.01 to 1 wt.-percent—calculated on the preparation.

15. A method for improving antimicrobial stability of a cosmetic or foodstuff preparation, comprising the step of adding a working amount of the composition of claim 1 to said preparation.

16. A method for masking unpleasant flavors, particularly unpleasant flavors of fluorine derivatives and bitter compounds, comprising the step of adding a working amount of the composition of claim 1 to said preparation.

17. A method for enhancing and/or boosting the performance of a flavor, a sweetener or a soothing agent, comprising the step of adding a working amount of the composition of claim 1 to said preparation.

18-20. (canceled)
